SEQUENCE LISTING (142 sequences)

SEQ ID NO: 1 - protein, 630 aa, Artificial Sequence
  F027300252
DVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSSGGGGSGGGSEVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSSGGGGSGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVKVSSA

SEQ ID NO: 2 - protein, 124 aa, Artificial Sequence
  1E07_BB1
DVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSS

SEQ ID NO: 3 - protein, 124 aa, Artificial Sequence
  1E07_BB2
EVQLVESGGGVVQPGGSLRLSCAASGRTFSSIYAKGWFRQAPGKEREFVAAISRSGRSTSYADSVKGRFTISRDNSKNTVYLQMNSLRPEDTALYYCAAVGGATTVTASEWDYWGQGTLVTVSS

SEQ ID NO: 4 - protein, 115 aa, Artificial Sequence
  1C02_BB3
EVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSS

SEQ ID NO: 5 - protein, 115 aa, Artificial Sequence
  ALB23002_BB4
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 6 - protein, 115 aa, Artificial Sequence
  1C02_BB5
EVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVKVSS

SEQ ID NO: 7 - protein, 11 aa, Artificial Sequence
  CDR1
GRTFSSIYAKG

SEQ ID NO: 8 - protein, 10 aa, Artificial Sequence
  CDR1
GFTFSDYWMY

SEQ ID NO: 9 - protein, 10 aa, Artificial Sequence
  CDR1
GFTFRSFGMS

SEQ ID NO: 10 - protein, 10 aa, Artificial Sequence
  CDR2
AISRSGRSTS

SEQ ID NO: 11 - protein, 10 aa, Artificial Sequence
  CDR2
EINTNGLITK

SEQ ID NO: 12 - protein, 10 aa, Artificial Sequence
  CDR2
SISGSGSDTL

SEQ ID NO: 13 - protein, 14 aa, Artificial Sequence
  CDR3
VGGATTVTASEWDY

SEQ ID NO: 14 - protein, 6 aa, Artificial Sequence
  CDR3
SPSGFN

SEQ ID NO: 15 - protein, 6 aa, Artificial Sequence
  CDR3
GGSLSR

SEQ ID NO: 16 - protein, 25 aa, Artificial Sequence
  FR1
DVQLVESGGGVVQPGGSLRLSCAAS

SEQ ID NO: 17 - protein, 25 aa, Artificial Sequence
  FR1
EVQLVESGGGVVQPGGSLRLSCAAS

SEQ ID NO: 18 - protein, 14 aa, Artificial Sequence
  FR2
WFRQAPGKEREFVA

SEQ ID NO: 19 - protein, 14 aa, Artificial Sequence
  FR2
WVRQAPGKGLEWVS

SEQ ID NO: 20 - protein, 14 aa, Artificial Sequence
  FR2
WVRQAPGKGPEWVS

SEQ ID NO: 21 - protein, 39 aa, Artificial Sequence
  FR3
YADSVKGRFTISRDNSKNTVYLQMNSLRPEDTALYYCAA

SEQ ID NO: 22 - protein, 39 aa, Artificial Sequence
  FR3
YPDSVKGRFTISRDNAKNTLYLQMNSLRPEDTALYYCAR

SEQ ID NO: 23 - protein, 39 aa, Artificial Sequence
  FR3
YADSVKGRFTISRDNSKNTLYLQMNSLRPEDTALYYCTI

SEQ ID NO: 24 - protein, 11 aa, Artificial Sequence
  FR4
WGQGTLVTVSS

SEQ ID NO: 25 - protein, 11 aa, Artificial Sequence
  FR4
RGQGTLVTVSS

SEQ ID NO: 26 - protein, 11 aa, Artificial Sequence
  FR4
SSQGTLVTVSS

SEQ ID NO: 27 - protein, 11 aa, Artificial Sequence
  FR4
RGQGTLVKVSS

SEQ ID NO: 28 - protein, 6 aa, Artificial Sequence
  CDR1
SIYAKG

SEQ ID NO: 29 - protein, 5 aa, Artificial Sequence
  CDR1
DYWMY

SEQ ID NO: 30 - protein, 5 aa, Artificial Sequence
  CDR1
SFGMS

SEQ ID NO: 31 - protein, 17 aa, Artificial Sequence
  CDR2
AISRSGRSTSYADSVKG

SEQ ID NO: 32 - protein, 17 aa, Artificial Sequence
  CDR2
EINTNGLITKYPDSVKG

SEQ ID NO: 33 - protein, 17 aa, Artificial Sequence
  CDR2
SISGSGSDTLYADSVKG

SEQ ID NO: 34 - protein, 30 aa, Artificial Sequence
  FR1
DVQLVESGGGVVQPGGSLRLSCAASGRTFS

SEQ ID NO: 35 - protein, 30 aa, Artificial Sequence
  FR1
EVQLVESGGGVVQPGGSLRLSCAASGRTFS

SEQ ID NO: 36 - protein, 30 aa, Artificial Sequence
  FR1
EVQLVESGGGVVQPGGSLRLSCAASGFTFS

SEQ ID NO: 37 - protein, 30 aa, Artificial Sequence
  FR1
EVQLVESGGGVVQPGGSLRLSCAASGFTFR

SEQ ID NO: 38 - protein, 14 aa, Artificial Sequence
  FR2
WFRQAPGKEREFVA

SEQ ID NO: 39 - protein, 14 aa, Artificial Sequence
  FR2
WVRQAPGKGLEWVS

SEQ ID NO: 40 - protein, 14 aa, Artificial Sequence
  FR2
WVRQAPGKGPEWVS

SEQ ID NO: 41 - protein, 32 aa, Artificial Sequence
  FR3
RFTISRDNSKNTVYLQMNSLRPEDTALYYCAA

SEQ ID NO: 42 - protein, 32 aa, Artificial Sequence
  FR3
RFTISRDNAKNTLYLQMNSLRPEDTALYYCAR

SEQ ID NO: 43 - protein, 32 aa, Artificial Sequence
  FR3
RFTISRDNSKNTLYLQMNSLRPEDTALYYCTI

SEQ ID NO: 44 - protein, 115 aa, Artificial Sequence [sequence omitted]

SEQ ID NO: 45 - protein, 115 aa, Artificial Sequence [sequence omitted]

SEQ ID NO: 46 - protein, 116 aa, Artificial Sequence
  Alb129
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 47 - protein, 116 aa, Artificial Sequence
  Alb132
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 48 - protein, 115 aa, Artificial Sequence [sequence omitted]

SEQ ID NO: 49 - protein, 116 aa, Artificial Sequence
  Alb11 (S112K)-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 50 - protein, 115 aa, Artificial Sequence
  Alb82
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 51 - protein, 116 aa, Artificial Sequence
  Alb82-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 52 - protein, 117 aa, Artificial Sequence
  Alb82-AA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 53 - protein, 118 aa, Artificial Sequence
  Alb82-AAA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 54 - protein, 116 aa, Artificial Sequence
  Alb82-G
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 55 - protein, 117 aa, Artificial Sequence
  Alb82-GG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 56 - protein, 118 aa, Artificial Sequence
  Alb82-GGG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 57 - protein, 115 aa, Artificial Sequence; identical to SEQ ID NO: 5
  Alb92

SEQ ID NO: 58 - protein, 116 aa, Artificial Sequence
  Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 59 - protein, 3 aa, Artificial Sequence
  3A linker
AAA

SEQ ID NO: 60 - protein, 5 aa, Artificial Sequence
  5GS linker
GGGGS

SEQ ID NO: 61 - protein, 7 aa, Artificial Sequence
  7GS linker
SGGSGGS

SEQ ID NO: 62 - protein, 8 aa, Artificial Sequence
  8GS linker
GGGGSGGS

SEQ ID NO: 63 - protein, 9 aa, Artificial Sequence
  9GS linker
GGGGSGGGS

SEQ ID NO: 64 - protein, 10 aa, Artificial Sequence
  10GS linker
GGGGSGGGGS

SEQ ID NO: 65 - protein, 15 aa, Artificial Sequence
  15GS linker
GGGGSGGGGSGGGGS

SEQ ID NO: 66 - protein, 18 aa, Artificial Sequence
  18GS linker
GGGGSGGGGSGGGGSGGS

SEQ ID NO: 67 - protein, 20 aa, Artificial Sequence
  20GS linker
GGGGSGGGGSGGGGSGGGGS

SEQ ID NO: 68 - protein, 25 aa, Artificial Sequence
  25GS linker
GGGGSGGGGSGGGGSGGGGSGGGGS

SEQ ID NO: 69 - protein, 30 aa, Artificial Sequence
  30GS linker
GGGGSGGGGSGGGGSGGGGSGGGGSGGGGS

SEQ ID NO: 70 - protein, 35 aa, Artificial Sequence
  35GS linker
GGGGSGGGGSGGGGSGGGGSGGGGSGGGGSGGGGS

SEQ ID NO: 71 - protein, 40 aa, Artificial Sequence
  40GS linker
GGGGSGGGGSGGGGSGGGGSGGGGSGGGGSGGGGSGGGGS

SEQ ID NO: 72 - protein, 15 aa, Artificial Sequence
  G1 hinge
EPKSCDKTHTCPPCP

SEQ ID NO: 73 - protein, 24 aa, Artificial Sequence
  9GS-G1 hinge
GGGGSGGGSEPKSCDKTHTCPPCP

SEQ ID NO: 74 - protein, 12 aa, Artificial Sequence
  Llama upper long hinge region
EPKTPKPQPAAA

SEQ ID NO: 75 - protein, 62 aa, Artificial Sequence
  G3 hinge
ELKTPLGDTTHTCPRCPEPKSCDTPPPCPRCPEPKSCDTPPPCPRCPEPKSCDTPPPCPRCP

SEQ ID NO: 76 - protein, 4 aa, Artificial Sequence
  Motif
KERE

SEQ ID NO: 77 - protein, 4 aa, Artificial Sequence
  Motif
KQRE

SEQ ID NO: 78 - protein, 4 aa, Artificial Sequence
  Motif
GLEW

SEQ ID NO: 79 - protein, 5 aa, Artificial Sequence
  Motif
KEREL

SEQ ID NO: 80 - protein, 5 aa, Artificial Sequence
  Motif
KEREF

SEQ ID NO: 81 - protein, 5 aa, Artificial Sequence
  Motif
KQREL

SEQ ID NO: 82 - protein, 5 aa, Artificial Sequence
  Motif
KQREF

SEQ ID NO: 83 - protein, 5 aa, Artificial Sequence
  Motif
KEREG

SEQ ID NO: 84 - protein, 5 aa, Artificial Sequence
  Motif
KQREW

SEQ ID NO: 85 - protein, 5 aa, Artificial Sequence
  Motif
KQREG

SEQ ID NO: 86 - protein, 4 aa, Artificial Sequence
  Motif
TERE

SEQ ID NO: 87 - protein, 5 aa, Artificial Sequence
  Motif
TEREL

SEQ ID NO: 88 - protein, 4 aa, Artificial Sequence
  Motif
TQRE

SEQ ID NO: 89 - protein, 5 aa, Artificial Sequence
  Motif
TQREL

SEQ ID NO: 90 - protein, 4 aa, Artificial Sequence
  Motif
KECE

SEQ ID NO: 91 - protein, 5 aa, Artificial Sequence
  Motif
KECEL

SEQ ID NO: 92 - protein, 5 aa, Artificial Sequence
  Motif
KECER

SEQ ID NO: 93 - protein, 4 aa, Artificial Sequence
  Motif
KQCE

SEQ ID NO: 94 - protein, 5 aa, Artificial Sequence
  Motif
KQCEL

SEQ ID NO: 95 - protein, 4 aa, Artificial Sequence
  Motif
RERE

SEQ ID NO: 96 - protein, 5 aa, Artificial Sequence
  Motif
REREG

SEQ ID NO: 97 - protein, 4 aa, Artificial Sequence
  Motif
RQRE

SEQ ID NO: 98 - protein, 5 aa, Artificial Sequence
  Motif
RQREL

SEQ ID NO: 99 - protein, 5 aa, Artificial Sequence
  Motif
RQREF

SEQ ID NO: 100 - protein, 5 aa, Artificial Sequence
  Motif
RQREW

SEQ ID NO: 101 - protein, 4 aa, Artificial Sequence
  Motif
QERE

SEQ ID NO: 102 - protein, 5 aa, Artificial Sequence
  Motif
QEREG

SEQ ID NO: 103 - protein, 4 aa, Artificial Sequence
  Motif
QQRE

SEQ ID NO: 104 - protein, 5 aa, Artificial Sequence
  Motif
QQREW

SEQ ID NO: 105 - protein, 5 aa, Artificial Sequence
  Motif
QQREL

SEQ ID NO: 106 - protein, 5 aa, Artificial Sequence
  Motif
QQREF

SEQ ID NO: 107 - protein, 4 aa, Artificial Sequence
  Motif
KGRE

SEQ ID NO: 108 - protein, 5 aa, Artificial Sequence
  Motif
KGREG

SEQ ID NO: 109 - protein, 4 aa, Artificial Sequence
  Motif
KDRE

SEQ ID NO: 110 - protein, 5 aa, Artificial Sequence
  Motif
KDREV

SEQ ID NO: 111 - protein, 5 aa, Artificial Sequence
  Motif
DECKL

SEQ ID NO: 112 - protein, 5 aa, Artificial Sequence
  Motif
NVCEL

SEQ ID NO: 113 - protein, 4 aa, Artificial Sequence
  Motif
GVEW

SEQ ID NO: 114 - protein, 4 aa, Artificial Sequence
  Motif
EPEW

SEQ ID NO: 115 - protein, 4 aa, Artificial Sequence
  Motif
GLER

SEQ ID NO: 116 - protein, 4 aa, Artificial Sequence
  Motif
DQEW

SEQ ID NO: 117 - protein, 4 aa, Artificial Sequence
  Motif
DLEW

SEQ ID NO: 118 - protein, 4 aa, Artificial Sequence
  Motif
GIEW

SEQ ID NO: 119 - protein, 4 aa, Artificial Sequence
  Motif
ELEW

SEQ ID NO: 120 - protein, 4 aa, Artificial Sequence
  Motif
GPEW

SEQ ID NO: 121 - protein, 4 aa, Artificial Sequence
  Motif
EWLP

SEQ ID NO: 122 - protein, 4 aa, Artificial Sequence
  Motif
GPER

SEQ ID NO: 123 - protein, 4 aa, Artificial Sequence
  Motif
GLER

SEQ ID NO: 124 - protein, 4 aa, Artificial Sequence
  Motif
ELEW

SEQ ID NO: 125 - protein, 5 aa, Artificial Sequence
  C-terminus
VTVSS

SEQ ID NO: 126 - protein, 5 aa, Artificial Sequence
  C-terminus
VKVSS

SEQ ID NO: 127 - protein, 5 aa, Artificial Sequence
  C-terminus
VQVSS

SEQ ID NO: 128 - protein, 6 aa, Artificial Sequence
  C-terminus
VTVSSA

SEQ ID NO: 129 - protein, 6 aa, Artificial Sequence
  C-terminus
VKVSSA

SEQ ID NO: 130 - protein, 6 aa, Artificial Sequence
  C-terminus
VQVSSA

SEQ ID NO: 131 - protein, 5 aa, Artificial Sequence
  C-terminus
VTVKS

SEQ ID NO: 132 - protein, 5 aa, Artificial Sequence
  C-terminus
VTVQS

SEQ ID NO: 133 - protein, 5 aa, Artificial Sequence
  C-terminus
VKVKS

SEQ ID NO: 134 - protein, 5 aa, Artificial Sequence
  C-terminus
VKVQS

SEQ ID NO: 135 - protein, 5 aa, Artificial Sequence
  C-terminus
VQVKS

SEQ ID NO: 136 - protein, 5 aa, Artificial Sequence
  C-terminus
VQVQS

SEQ ID NO: 137 - protein, 6 aa, Artificial Sequence
  C-terminus
VTVKSA

SEQ ID NO: 138 - protein, 6 aa, Artificial Sequence
  C-terminus
VTVQSA

SEQ ID NO: 139 - protein, 6 aa, Artificial Sequence
  C-terminus
VKVKSA

SEQ ID NO: 140 - protein, 6 aa, Artificial Sequence
  C-terminus
VKVQSA

SEQ ID NO: 141 - protein, 6 aa, Artificial Sequence
  C-terminus
VQVKSA

SEQ ID NO: 142 - protein, 6 aa, Artificial Sequence
  C-terminus
VQVQSA

(19) United States
(12) Patent Application Publication
(10) Pub. No.: US 2021/0188986 A1
(43) Pub. Date: Jun. 24, 2021

(54) POLYPEPTIDES COMPRISING IMMUNOGLOBULIN SINGLE VARIABLE DOMAINS TARGETING TNFA AND OX40L

(71) Applicants: Ablynx N.V., Ghent-Zwijnaarde (BE); Sanofi, Paris (FR)

(72) Inventors: Heidi Rommelaere, Gent (BE); Ann Brigé, Ertvelde (BE); Sigrid Cornelis, St. Martens-Latem (BE); Peter Florian, Frankfurt am Main (DE); Karen Heyninck, Zwijnaarde (BE); Thomas Kreutzberg, Frankfurt am Main (DE); Thomas Leeuw, Frankfurt am Main (DE); Eric Lorent, Zwijnaarde (BE); Oezen Sercan Alp, Frankfurt am Main (DE); Gertrud Sibenhorn, Frankfurt am Main (DE)

(73) Assignees: Ablynx N.V., Ghent-Zwijnaarde (BE); Sanofi, Paris (FR)

(21) Appl. No.: 17/111,671

(22) Filed: Dec. 4, 2020

Related U.S. Application Data
(60) Provisional application No. 62/944,661, filed on Dec. 6, 2019.

(30) Foreign Application Priority Data
Jan. 28, 2020 (EP) 20305071.1

(51) Int. Cl. C07K 16/28; A61P 29/00; C07K 16/24

(52) U.S. Cl. CPC C07K 16/2875; A61P 29/00; C07K 16/241; C07K 2317/14; C07K 2319/30; C07K 2317/565; C07K 2317/94; C07K 2319/31

(57) ABSTRACT

The present technology aims at providing a novel type of drug for treating a subject suffering from an autoimmune or inflammatory disease. Specifically, the present technology provides polypeptides comprising at least four immunoglobulin single variable domains (ISVDs), characterized in that at least two ISVDs bind to TNFα and at least two ISVDs binds to OX4OL. The present technology also provides nucleic acids, vectors and compositions.

### RELATED APPLICATION

This application claims the benefit under 35 U.S.C. § 119(e) of U.S. provisional application Ser. No. 62/944,661, filed Dec. 6, 2019, the entire contents of which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 3, 2020, is named A0848.70214US01-SEQ-JRV, and is 48 kilobytes in size.

### DESCRIPTION

### 1 FIELD OF THE PRESENT TECHNOLOGY

The present technology relates to polypeptides targeting TNFα and OX40L. It also relates to nucleic acid molecules encoding the polypeptide and vectors comprising the nucleic acids, and to compositions comprising the polypeptide, nucleic acid or vector. The present technology further relates to these products for use in a method of treating a subject suffering from an autoimmune or inflammatory disease. Moreover, the present technology relates to method of producing these products.

### 2 TECHNOLOGICAL BACKGROUND

Autoimmune or inflammatory diseases are the result of an immune response produced by a body against its own tissue. Autoimmune or inflammatory diseases are often chronic and can even be life-threatening. Autoimmune or inflammatory diseases include inflammatory bowel disease, such as Crohn's disease and ulcerative colitis, rheumatoid arthritis, psoriasis, psoriatic arthritis, and hidradenitis suppurativa. Inflammatory bowel disease, such as Crohn's disease and ulcerative colitis, is a chronic inflammatory disease involving intestinal inflammation and concomitant epithelial injury. Other chronic autoimmune diseases, such as psoriasis, psoriatic arthritis and hidradenitis suppurativa, are characterized by patches of red, dry, itchy or scaly skin, painful inflammation of joints or inflamed and swollen lumps on the skin. It has been found that patients suffering from psoriasis are more likely to have certain comorbidities, including diabetes and inflammatory bowel disease, such as Crohn's disease or ulcerative colitis, and cancer.

Tumor Necrosis Factor alpha (TNFα) is a homotrimeric cytokine which is produced mainly by monocytes and macrophages, but also known to be secreted by CD4⁺ and CD8⁺ peripheral blood T lymphocytes. TNFα can exist as a soluble form or as a transmembrane protein. The primary role of TNFα is in the regulation of immune cells. TNFα acts as an endogenous pyrogen and dysregulation of its production has been implicated in a variety of human diseases including Rheumatoid Arthritis (RA), Psoriasis (Pso), Hidradenitis Suppurativa (HS), Inflammatory Bowel Disease (IBD) such as Crohn's disease (CD) and ulcerative colitis (UC), and graft-versus-host disease (GVHD).

Treatments currently approved by the FDA for RA and IBD inflammatory bowel disease include anti-TNFα biologicals (such as Simponi® [golimumab], Enbrel® [etanercept], Remicade® [infliximab] and Humira® [adalimumab]). However, current anti-TNFα treatments for RA only show a full disease remission in a minority of patients and a substantial portion of non-responders is still remaining. Similarly, current anti-TNFα treatments for inflammatory bowel disease face a large percentage of patients being non-responsive to currently available treatments, and loss of response to anti-TNFα treatment occurs in a high percentage of patients following 12 months of treatment. For psoriasis and psoriatic arthritis, only a minority of patients is treated with biologicals including Remicade® [infliximab] and Humira® [adalimumab]. Current treatments have been shown to be efficacious for some treating psoriasis, at least in a subset of patients.

Thus, for autoimmune diseases such as e.g. rheumatoid arthritis or psoriatic arthritis so far no biological has exhibited sufficient efficacy with respect to disease remission in a significant percentage of patients and lack of, or loss of, response is still an issue. OX40L (also known as CD252 or TNFSF4) is a member of the TNF superfamily and is the inducible co-stimulatory ligand for the OX40 receptor (also known as CD134 or TNFRSF4). It is expressed mainly on activated antigen-presenting cells (APCs) including dendritic cells, macrophages, and B cells. OX40 on the other hand is largely expressed on activated T cells and natural killer T cells. OX40L is mostly expressed as membrane-bound molecule but can also be detected in a cleaved soluble form. OX40L/OX40 has been recognized as an immune co-stimulatory regulator in a number of diseases that are characterized by activated T-cells which orchestrate the immune response. It triggers signalling through OX40, resulting in a range of activities including production and release of inflammatory cytokines, expansion and accumulation of effector T cells (e.g. TH1, TH2, TH17) and cytotoxic T cells, as well as decreasing the suppressive efficacy of Treg cells. Although several studies suggest an implication of the co-stimulatory OX40L/OX40 axis in autoimmune diseases such as RA, Pso, or IBD, there is currently no FDA-approved OX-40L biological for their treatment.

Targeting multiple disease factors may be achieved for example by co-administration or combinatorial use of two separate biologicals, e.g. antibodies binding to different therapeutic targets. However, co-administration or combinatorial use of separate biologicals can be challenging, both from a practical and a commercial point of view. For example, two injections of separate products result in a more inconvenient and more painful treatment regime to the patients which may negatively affect compliance. With regard to a single injection of two separate products, it can be difficult or impossible to provide formulations that allow for acceptable viscosity at the required concentrations and suitable stability of both products. Additionally, co-administration and co-formulation requires production of two separate drugs, which can increase overall costs.

Bispecific antibodies that are able to bind to two different antigens have been suggested as one strategy for addressing such limitations associated with co-administration or combinatorial use of separate biologicals, such as antibodies.

Bispecific antibody constructs have been proposed in multiple formats. For example, bispecific antibody formats may involve the chemical conjugation of two antibodies or fragments thereof (Brennan, M, et al., Science, 1985. 229(4708): p. 81-83; Glennie, M. J., et al., J Immunol, 1987. 139(7): p. 2367-2375).

Disadvantages of such bispecific antibody formats include, however, high viscosity at high concentration, making e.g. subcutaneous administration challenging, and in that each binding unit requires the interaction of two variable domains for specific and high affinity binding, having implications on polypeptide stability and efficiency of production. Such bispecific antibody formats may also potentially lead to Chemistry, Manufacturing and Control (CMC) issues related to mispairing of the light chains or mispairing of the heavy chains.

### 3 SUMMARY OF THE PRESENT TECHNOLOGY

In some embodiments, the present technology relates to a polypeptide (or ISVD construct) targeting specifically TNFα and OX40L at the same time leads to an increased efficiency of modulating an inflammatory response as compared to monospecific anti-TNFα or anti-OX40L polypeptides.

In some embodiments, the polypeptides of the present technology are efficiently produced (e.g. in microbial hosts) and have low viscosity at high concentrations which is advantageous and convenient for subcutaneous administration. Furthermore, in some embodiments, the polypeptides of the present technology have limited reactivity to pre-existing antibodies in the subject to be treated (i.e. antibodies present in the subject before the first treatment with the antibody construct). In preferred embodiments such polypeptides exhibit a half-life in the subject to be treated that is long enough such that consecutive treatments can be conveniently spaced apart.

The polypeptide of the present technology comprises or consists of at least four immunoglobulin single variable domains (ISVDs), wherein at least two ISVDs specifically bind to TNFα and at least two ISVDs specifically bind to OX40L. Preferably, the at least two ISVDs binding to TNFα specifically bind to human TNFα and the at least two ISVDs binding to OX40L specifically bind to human OX40L.

The polypeptide preferably further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units. For example, the binding unit can be an ISVD that binds to a serum protein, preferably to a human serum protein such as human serum albumin.

Also provided is a nucleic acid molecule capable of expressing the polypeptide of the present technology, a nucleic acid or vector comprising the nucleic acid, and a composition comprising the polypeptide, the nucleic acid or the vector. The composition is preferably a pharmaceutical composition.

Also provided is a host or host cell comprising the nucleic acid or vector that encodes the polypeptide according to the present technology.

Further provided is a method for producing the polypeptide according to present technology, said method at least comprising the steps of:
a. expressing, optionally in a suitable host cell or host organism or in another suitable expression system, a nucleic acid sequence encoding the polypeptide according to the present technology optionally followed by:
b. isolating and/or purifying the polypeptide according to the present technology.

Moreover, the present technology provides the polypeptide, the composition comprising the polypeptide, or the composition comprising the nucleic acid or vector comprising the nucleotide sequence that encodes the polypeptide, for use as a medicament. Preferably, the polypeptide or composition is for use in the treatment of an autoimmune or an inflammatory disease, wherein preferably the autoimmune or inflammatory disease is selected from rheumatoid arthritis, inflammatory bowel disease, such as Crohn's disease and ulcerative colitis, psoriasis, Hidradenitis suppurativa, graft-versus-host disease.

In addition, provided is a method of treating an autoimmune disease or an inflammatory disease, wherein said method comprises administering, to a subject in need thereof, a pharmaceutically active amount of the polypeptide or a composition according to the present technology. The autoimmune disease or inflammatory disease is preferably selected from rheumatoid arthritis, inflammatory bowel disease, such as Crohn's disease and ulcerative colitis, and Hidradenitis suppurativa. In a preferred embodiment, the method further comprises administering one or more additional therapeutic agents, such as methotrexate.

Further provided is the use of the polypeptide or composition of the present technology in the preparation of a pharmaceutical composition for treating an autoimmune disease or an inflammatory disease, wherein the autoimmune disease or inflammatory disease is preferably selected from rheumatoid arthritis, inflammatory bowel disease, such as Crohn's disease and ulcerative colitis, psoriasis, Hidradenitis suppurativa, graft-versus-host disease.

In particular, the present technology provides the following embodiments:

Embodiment 1: A polypeptide, a composition comprising the polypeptide, or a composition comprising a nucleic acid comprising a nucleotide sequence that encodes the polypeptide, for use as a medicament, wherein the polypeptide comprises or consists of at least four immunoglobulin single variable domains (ISVDs), wherein each of said ISVDs comprises three complementarity determining regions (CDR1 to CDR3, respectively), optionally linked via one or more peptidic linkers; and wherein:
a. a first ISVD and a second ISVD comprises
   i. a CDR1 comprising the amino acid sequence of SEQ ID NO: 7 or has 2 or 1 amino acid difference(s) with SEQ ID NO: 7;
   ii. a CDR2 comprising the amino acid sequence of SEQ ID NO: 10 or has 2 or 1 amino acid difference(s) with SEQ ID NO: 10; and
   iii. a CDR3 comprising the amino acid sequence of SEQ ID NO: 13 or has 2 or 1 amino acid difference(s) with SEQ ID NO: 13; and
b. a third ISVD and a fourth ISVD comprises
   iv. a CDR1 comprising the amino acid sequence of SEQ ID NO: 8 or has 2 or 1 amino acid difference(s) with SEQ ID NO: 8;
   v. a CDR2 comprising the amino acid sequence of SEQ ID NO: 11 or has 2 or 1 amino acid difference(s) with SEQ ID NO: 11; and
   vi. a CDR3 comprising the amino acid sequence of SEQ ID NO: 14 or has 2 or 1 amino acid difference(s) with SEQ ID NO: 14,

wherein the ISVDs are in the order starting from the N-terminus.

Embodiment 2: The composition for use according to embodiment 1, which is a pharmaceutical composition which further comprises at least one pharmaceutically acceptable carrier, diluent or excipient and/or adjuvant, and optionally comprises one or more further pharmaceutically active polypeptides and/or compounds.

Embodiment 3: The polypeptide or composition for use according to embodiment 1 or 2, wherein:
a. said first ISVD and said second ISVD comprises a CDR1 comprising the amino acid sequence of SEQ ID NO: 7, a CDR2 comprising the amino acid sequence of SEQ ID NO: 10 and a CDR3 comprising the amino acid sequence of SEQ ID NO: 13; and
b. said third ISVD and said fourth ISVD comprises a CDR1 comprising the amino acid sequence of SEQ ID NO: 8, a CDR2 comprising the amino acid sequence of SEQ ID NO: 11 and a CDR3 comprising the amino acid sequence of SEQ ID NO: 14.

Embodiment 4: The polypeptide or composition for use according to any of embodiments 1 to 3, wherein:
a. the amino acid sequence of said first ISVD comprises a sequence identity of more than 90% with SEQ ID NO: 2;
b. the amino acid sequence of said second ISVD comprises a sequence identity of more than 90% with SEQ ID NO: 3;
c. the amino acid sequence of said third ISVD comprises a sequence identity of more than 90% identity with SEQ ID NO: 4; and
d. the amino acid sequence of said fourth ISVD comprises a sequence identity of more than 90% identity with SEQ ID NO: 6.

Embodiment 5: The polypeptide or composition for use according to any of embodiments 1 to 4, wherein:
a. said first ISVD comprises the amino acid sequence of SEQ ID NO: 2;
b. said second ISVD comprises the amino acid sequence of SEQ ID NO: 3;
c. said third ISVD comprises the amino acid sequence of SEQ ID NO: 4; and
d. said fourth ISVD comprises the amino acid sequence of SEQ ID NO: 6.

Embodiment 6: The polypeptide or composition for use according to any of embodiments 1 to 5, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

Embodiment 7: The polypeptide or composition for use according to embodiment 6 in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.

Embodiment 8: The polypeptide or composition for use according to any one of embodiments 6 to 7, in which said one or more other groups, residues, moieties or binding units that provide the polypeptide with increased half-life is chosen from the group consisting of binding units that can bind to serum albumin (such as human serum albumin) or a serum immunoglobulin (such as IgG).

Embodiment 9: The polypeptide or composition for use according to embodiment 8, in which said binding unit that provides the polypeptide with increased half-life is an ISVD that can bind to human serum albumin.

Embodiment 10: The polypeptide or composition for use according to embodiment 9, wherein the ISVD binding to human serum albumin comprises
i. a CDR1 comprising the amino acid sequence of SEQ ID NO: 9 or has 2 or 1 amino acid difference(s) with SEQ ID NO: 9;
ii. a CDR2 comprising the amino acid sequence of SEQ ID NO: 12 or has 2 or 1 amino acid difference(s) with SEQ ID NO: 12; and
iii. a CDR3 comprising the amino acid sequence of SEQ ID NO: 15 or has 2 or 1 amino acid difference(s) with SEQ ID NO: 15.

Embodiment 11: The polypeptide or composition for use according to any of embodiments 9 to 10, wherein the ISVD binding to human serum albumin comprises a CDR1 comprising the amino acid sequence of SEQ ID NO: 9, a CDR2 comprising the amino acid sequence of SEQ ID NO: 12 and a CDR3 comprising the amino acid sequence of SEQ ID NO: 15.

Embodiment 12: The polypeptide or composition for use according to any of embodiments 9 to 11, wherein the amino acid sequence of said ISVD binding to human serum albumin comprises a sequence identity of more than 90% with SEQ ID NO: 5.

Embodiment 13: The polypeptide or composition for use according to any of embodiments 9 to 12, wherein said ISVD binding to human serum albumin comprises the amino acid sequence of SEQ ID NO: 5.

Embodiment 14: The polypeptide or composition for use according to any of embodiments 1 to 13, wherein the amino acid sequence of the polypeptide comprises a sequence identity of more than 90% with SEQ ID NO: 1.

Embodiment 15: The polypeptide or composition for use according to any of embodiments 1 to 14, wherein the polypeptide comprises or consists of the amino acid sequence of SEQ ID NO: 1.

Embodiment 16: The polypeptide or composition for use according to any of embodiments 1 to 15, for use in the treatment of an autoimmune or an inflammatory disease.

Embodiment 17: The polypeptide or composition for use according to embodiment 16, wherein the autoimmune or inflammatory disease is selected from rheumatoid arthritis, inflammatory bowel disease, such as Crohn's disease and ulcerative colitis, psoriasis, Hidradenitis suppurativa, and graft-versus-host disease.

Embodiment 18: A polypeptide comprising nucleic acid comprising a nucleotide sequence that encodes the polypeptide, wherein the polypeptide comprises or consists of at least four immunoglobulin single variable domains (ISVDs), wherein each of said ISVDs comprises three complementarity determining regions (CDR1 to CDR3, respectively), optionally linked via one or more peptidic linkers; and wherein:
a. a first ISVD and a second ISVD comprises
   i. a CDR1 comprising the amino acid sequence of SEQ ID NO: 7 or has 2 or 1 amino acid difference(s) with SEQ ID NO: 7;
   ii. a CDR2 comprising the amino acid sequence of SEQ ID NO: 10 or has 2 or 1 amino acid difference(s) with SEQ ID NO: 10; and
   iii. a CDR3 comprising the amino acid sequence of SEQ ID NO: 13 or has 2 or 1 amino acid difference(s) with SEQ ID NO: 13; and
b. a third ISVD and a fourth ISVD comprises
   iv. a CDR1 comprising the amino acid sequence of SEQ ID NO: 8 or has 2 or 1 amino acid difference(s) with SEQ ID NO: 8;
   v. a CDR2 comprising the amino acid sequence of SEQ ID NO: 11 or has 2 or 1 amino acid difference(s) with SEQ ID NO: 11; and
   vi. a CDR3 comprising the amino acid sequence of SEQ ID NO: 14 or has 2 or 1 amino acid difference(s) with SEQ ID NO: 14,

wherein the ISVDs are in the order starting from the N-terminus.

Embodiment 19: The polypeptide according to embodiment 18, wherein:
a. said first ISVD and said second ISVD comprises a CDR1 comprising the amino acid sequence of SEQ ID NO: 7, a CDR2 comprising the amino acid sequence of SEQ ID NO: 10 and a CDR3 comprising the amino acid sequence of SEQ ID NO: 13; and
b. said third ISVD and said fourth ISVD comprises a CDR1 comprising the amino acid sequence of SEQ ID NO: 8, a CDR2 comprising the amino acid sequence of SEQ ID NO: 11 and a CDR3 comprising the amino acid sequence of SEQ ID NO: 14.

Embodiment 20: The polypeptide according to embodiment 18 or 19, wherein:
a. the amino acid sequence of said first ISVD comprises a sequence identity of more than 90% with SEQ ID NO: 2;
b. the amino acid sequence of said second ISVD comprises a sequence identity of more than 90% with SEQ ID NO: 3;
c. the amino acid sequence of said third ISVD comprises a sequence identity of more than 90% identity with SEQ ID NO: 4; and
d. the amino acid sequence of said fourth ISVD comprises a sequence identity of more than 90% identity with SEQ ID NO: 6.

Embodiment 21: The polypeptide according to any of embodiments 18 to 20, wherein:
a. said first ISVD comprises the amino acid sequence of SEQ ID NO: 2;
b. said second ISVD comprises the amino acid sequence of SEQ ID NO: 3;
c. said third ISVD comprises the amino acid sequence of SEQ ID NO: 4; and
d. said fourth ISVD comprises the amino acid sequence of SEQ ID NO: 6.

Embodiment 22: The polypeptide according to any of embodiments 18 to 21, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

Embodiment 23: The polypeptide according to embodiment 22 in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.

Embodiment 24: The polypeptide according to any one of embodiments 22 to 23, in which said one or more other groups, residues, moieties or binding units that provide the polypeptide with increased half-life is chosen from the group consisting of binding units that can bind to serum albumin (such as human serum albumin) or a serum immunoglobulin (such as IgG).

Embodiment 25: The polypeptide according to embodiment 24, in which said binding unit that provides the polypeptide with increased half-life is an ISVD that can bind to human serum albumin.

Embodiment 26: The polypeptide according to embodiment 25, wherein the ISVD binding to human serum albumin comprises
i. a CDR1 comprising the amino acid sequence of SEQ ID NO: 9 or has 2 or 1 amino acid difference(s) with SEQ ID NO: 9;
ii. a CDR2 comprising the amino acid sequence of SEQ ID NO: 12 or has 2 or 1 amino acid difference(s) with SEQ ID NO: 12; and
iii. a CDR3 comprising the amino acid sequence of SEQ ID NO: 15 or has 2 or 1 amino acid difference(s) with SEQ ID NO: 15.

Embodiment 27: The polypeptide or composition for use according to any of embodiments 25 to 26, wherein the ISVD binding to human serum albumin comprises a CDR1 comprising the amino acid sequence of SEQ ID NO: 9, a CDR2 comprising the amino acid sequence of SEQ ID NO: 12 and a CDR3 comprising the amino acid sequence of SEQ ID NO: 15.

Embodiment 28: The polypeptide or composition for use according to any of embodiments 25 to 27, wherein the amino acid sequence of said ISVD binding to human serum albumin comprises a sequence identity of more than 90% with SEQ ID NO: 5.

Embodiment 29: The polypeptide according to any of embodiments 25 to 28, wherein said ISVD binding to human serum albumin comprises the amino acid sequence of SEQ ID NO: 5.

Embodiment 30: The polypeptide according to any of embodiments 18 to 29, wherein the amino acid sequence of the polypeptide comprises a sequence identity of more than 90% with SEQ ID NO: 1.

Embodiment 31: The polypeptide or composition for use according to any of embodiments 18 to 30, wherein the polypeptide comprises or consists of the amino acid sequence of SEQ ID NO: 1.

Embodiment 32: A nucleic acid comprising a nucleotide sequence that encodes a polypeptide according to any of embodiments 18 to 31.

Embodiment 33: A host or host cell comprising a nucleic acid according to embodiment 32.

Embodiment 34: A method for producing a polypeptide according to any of embodiments 18-31, said method at least comprising the steps of:
a. expressing, in a suitable host cell or host organism or in another suitable expression system, a nucleic acid according to embodiment 32; optionally followed by:
b. isolating and/or purifying the polypeptide according to any of embodiments 18 to 31.

Embodiment 35: A composition comprising at least one polypeptide according to any of embodiments 18 to 31, or a nucleic acid according to embodiment 32.

Embodiment 36: The composition according to embodiment 35, which is a pharmaceutical composition which further comprises at least one pharmaceutically acceptable carrier, diluent or excipient and/or adjuvant, and optionally comprises one or more further pharmaceutically active polypeptides and/or compounds.

Embodiment 37: A method of treating an autoimmune disease or an inflammatory disease, wherein said method comprises administering, to a subject in need thereof, a pharmaceutically active amount of a polypeptide according to any of embodiments 18 to 31 or a composition according to any of embodiments 35 to 36.

Embodiment 38: The method according to embodiment 37, wherein the autoimmune disease or inflammatory disease is selected from rheumatoid arthritis, inflammatory bowel disease, such as Crohn's disease and ulcerative colitis, psoriasis, Hidradenitis suppurativa, graft-versus-host disease.

Embodiment 39: The method according to any of embodiments 37 to 38, wherein the method further comprises administering one or more additional therapeutic agents.

Embodiment 40: The method according to embodiment 39, wherein the additional therapeutic agent is methotrexate.

Embodiment 41: Use of a polypeptide according to any of embodiments 18 to 31 or a composition according to any of embodiments 35 to 36, in the preparation of a pharmaceutical composition for treating an autoimmune disease or an inflammatory disease.

Embodiment 42: Use of the polypeptide or composition according to embodiment 41, wherein the autoimmune disease or inflammatory disease is selected from rheumatoid arthritis, inflammatory bowel disease, such as Crohn's disease and ulcerative colitis, psoriasis, Hidradenitis suppurativa, graft-versus-host disease.

### 4 BRIEF DESCRIPTION OF THE DRAWINGS

FIG. 1: Sensorgram showing simultaneous binding of recombinant soluble hTNFα and hOX40L to ISVD construct F027300252 captured via HSA.

FIG. 2: Simultaneous binding of soluble TNFα and membrane bound hOX40L to ISVD construct F027300252 as shown by flow cytometry on CHO-Ki cells expressing human OX40L. IRR00096 is a negative control V_{HH}.

FIG. 3: Inhibition of soluble human and cyno TNFα in the Glo responseTM HEK293_NFκB-NLucP reporter assay by ISVD construct F0275000252 and the reference compound anti-hTNFα reference mAb, IRR00096 is a negative control V_{HH}.

FIG. 4: Inhibition of membrane bound OX40L by ISVD construct F0275000252 and the reference compound anti-hOX40L mAb as determined in the PBMC activity assay.

FIG. 5: Induction of luciferase activity [RLU] by recombinant human TNFα at 5 ng/ml or human OX40L at 100 ng/ml, or a combination of both, and inhibition of induced luciferase activity by an anti-TNFα antibody (PB03017; from Sanofi), an anti-OX40L antibody (Cat #AB00536 from Absolute Antibody), or a combination of both antibodies at different concentrations from 0.5 μg/ml up to 5μg/ml.

FIG. 6: Induction of luciferase activity [RLU] by a combination of recombinant human TNFα and human OX40L (as explained in FIG. 5), and inhibition of induced luciferase activity by the monospecific anti-OX40L V_{HH} ALX-0632, or the monospecific anti-TNF V_{HH} ATN-103, or the anti-TNF/anti-OX40L bispecific ISVD constructs F027300252, F027301104, F027301189, F027301197, and F027301199. Shown is the % maximum inhibition of NFκB luciferase activity achieved by bispecific or monospecific ISVD constructs/V_{HH} s.

FIG. 7: Induction of luciferase activity by a combination of recombinant human TNFα and human OX40L (as explained in FIG. 5), and inhibition of induced luciferase activity by the anti-TNF/anti-OX40L bispecific ISVD constructs F027300252, F027301104, F027301189, F027301197, and F027301199. Shown are the mean IC50 values (pM)±SD from at least 3 independent experiments.

FIG. 8: Surface expression of OX40L on human monocyte-derived dendritic cells at days 1, 2 and 3 of maturation. The expression of OX40L was measured by flow cytometry. Results correspond to the mean±SEM from 3 different human mDC donors tested against PBMC's from 5 allogeneic donors.

FIG. 9: GM-CSF expression in the MLR assay at day 5. The secretion of GM-CSF was measured in the supernatant of the MLR assay after incubation with anti-TNFα antibody alone [10 μg/ml], or anti-OX40L antibody alone [10 μg/ml], or a combination of anti-TNF [10 μg/ml]+anti-OX40L [10 μg/ml] antibodies. Results from 5 PBMC donors tested against DC from 3 allogeneic DC donors. **p<0.0016, ****p<0.0001.

FIG. 10: box plot showing the binding of pre-existing antibodies present in 96 human serum samples to ISVD constructs F027300252, F027301104, F027301189 and F027301197 compared to control ISVD constructs F027301099 and F027301186.

FIG. 11: box plot showing the binding of pre-existing antibodies present in 96 human serum samples to ISVD constructs F027300028, F027300252, F027301097 and F027301186

FIG. 12: Arthritis score over time in different treatment groups (n=8 mice/group). Animals received intraperitoneal injections of the indicated compounds twice per week, starting at 6 weeks of age. Shown are mean weekly arthritis scores±SEM. Statistics are 2-way ANOVA and Bonferroni multiple comparison test. ns (not significant) p>0.05, * p<0.05, ** p<0.01. *** p<0.001, **** p<0.0001.

FIG. 13: Area under the curve for arthritis score over time. Shown are individual values (symbols) and means±SEM (bars). Statistics are 1-way ANOVA and Bonferroni multiple comparison test. ns (not significant) p>0.05, * p<0.05, ** p<0.01. *** p<0.001, **** p<0.0001.

FIG. 14: Histology score. Shown are individual values (symbols) and means±SEM (bars). Statistics are 1-way ANOVA and Bonferroni multiple comparison test.

FIG. 15: Study design for testing the efficacy of anti-TNFa-OX40L ISVD construct F027300252 (referred to as nAb) in a TNFα humanized mouse collagen antibody induced arthritis (CAIA) model. Anti-hTNFα reference mAb was dosed at the same time as the ISVD construct, 1^{st} dose 6 hours post LPS and 2nd dose 3 days after LPS on day 4.

FIG. 16: Arthritis score development over time of the experiment (n=8 mice/group apart from vehicle: n=16/group from two independent experiments) in a mouse model of CAIA. Animals received two intraperitoneal injections of either vehicle, the reference compound Anti-hTNFα reference mAb, or the half-life extended anti-TNFa-OX40L ISVD construct F027300252 according to the scheme in FIG. 10. Statistics are 2-way ANOVA and Bonferroni multiple comparison test.

FIG. 17: AUC of the arthritis score from the experiment (n=8 mice/group apart from vehicle: n=16/group from two independent experiments) in a mouse model of CAIA.

FIG. 18: Study scheme for the combined TDAR-DTH model. Light grey boxes on top of the time course in days illustrate the KLH administration for the TDAR part of the study. The dark grey boxed mark the intramuscular injection of the second antigen tetanoid toxin (TTx) with Aluminium hydroxide (ALU) for the DTH part of the study. White boxes on top of the time course mark the skin challenge with TTx/ALU and KLH on day 31 and day 56 with a dark arrow for the DTH model. Skin biopsies that were assessed for histopathology and immunohistochemistry are shown for day 34 and at necropsy on day 59. After the TTx/ALU and KLH challenge the skin area was followed up 24/48/72 hours at the respective DTH challenge to assess in-life changes as described in table 15.

FIG. 19: Mean Serum concentrations (ng/mL) of F027300252 after subcutaneous administration on Day 1 and 29 at 3, 10, 30, 100 (mg/kg/adm) to female monkeys (semi-log scale plot).

FIG. 20: Anti-KLH response in the cynomolgus monkey TDAR with a focus on anti-KLH IgG response. For each group 4 cynomolgus monkeys were used. Data is depicted as mean±SD. D3 (day 3) and d31 (day 31) mark the timepoints for KLH stimulation. The primary response spans the time window between BL (−12D) and day 30 (30D), the secondary response between day 31 (31D) and the end of the study at day 59 (59D). F027300252 was administered in a dose-dependent fashion from 3 mg/kg to 100 mg/kg via weekly subcutaneous injections. Treatment stopped with the fifth injection of day 29. Statistics are 2-way ANOVA and Bonferroni multiple comparison test and was applied to the secondary response. During the primary response all treatment groups are not significantly different to vehicle.

FIG. 21: Mean reduction in anti KLH IgG AUC during secondary response of the TDAR between day 34 and day 59 in percentage from vehicle of anti-hTNFα reference mAb at 4 mg/kg and anti-hOX40L reference mAb at 8 mg/kg (pilot study) and from different dose levels of F027300252 (3, 10, 30, and 100 mg/kg) in TDAR monkey study. Doses were expressed in nmol per kg.

FIG. 22: IFN-y spot forming cells/million of cells after KLH re-stimulation of PBMCs on day 59 determined by an ELISPOT assay. The bar represents the mean value±SD. Individual animals are depicted by either closed circles (vehicle control) or open circles (F027300252 treated groups). The respective dose is provided in mg/kg for F027300252. In the group at 30 mg/kg for one animal the assay did not fulfill the quality control standards so that only three animals out of four are shown. Statistics are 1-way ANOVA and Bonferroni multiple comparison test.

FIG. 23: IL-4 spot forming cells/million of cells after KLH re-stimulation of PBMCs on day 59 determined by an ELISPOT assay. The bar represents the mean value±SD. Individual animals are depicted by either closed circles (vehicle control) or open circles (F027300252 treated groups). The respective dose is provided in mg/kg for F027300252. In the group at 30 mg/kg for one animal the assay did not fulfill the quality control standards so that only three animals out of four are shown. Statistics are 1-way ANOVA and Bonferroni multiple comparison test.

FIG. 24: Schematic drawing of the different immunizations for either TDAR (light grey) and DTH (dark grey) on the left of the cartoon. For DTH the animals were challenged either on day 31 or day 59 with TTX/ALU and KLH, respectively. Different intradermal injection sites were used as depicted in the middle cartoon. Biopsies of 8 mm in diameter were taken at day 34 and at the end of the study on day 59 and assessed by histopathology and immunohistochemistry (right part of the cartoon).

FIG. 25: GVHD score development over time in the xeno-GVHD mouse model. Data are depicted as mean±SEM. N=7, 2 different hPBMC donors. Statistics are 1-way ANOVA and Bonferroni's multiple comparison test.

FIG. 26: Survival over time in the xeno-GVHD mouse model. Data are depicted as Kaplan-Meier survival curve. n=7, 2 different hPBMC donors. Survival data were analyzed using Log-rank (Mantel-Cox) test. P values were corrected for multiple comparisons.

FIG. 27: Engraftment of hPBMCs in recipient NSG mice. Data are depicted as mean±SEM. n=7, 2 different hPBMC donors. Kaplan-Engraftment data were analyzed using Mixed-effects analysis and Bonferroni's multiple comparison test.

FIG. 28: GVHD score development over time in the xeno-GVHD mouse model. Data are depicted as mean±SEM. Results were pooled from two independent studies. n=7-12, 3 different hPBMC donors. Statistics are 1-way ANOVA and Bonferroni's multiple comparison test.

FIG. 29: Survival over time in the xeno-GVHD mouse model. Data are depicted as Kaplan-Meier survival curve. Results were pooled from two independent studies. n=7-12, 3 different hPBMC donors. Survival data were analyzed using Log-rank (Mantel-Cox) test. P values were corrected for multiple comparisons.

FIG. 30: Engraftment of hPBMCs in host NSG mice. Data are depicted as mean±SEM. Results were pooled from two independent studies. n=7-12, 3 different hPBMC donors. Engraftment data were analyzed using Mixed-effects analysis and Bonferroni's multiple comparison test.

FIG. 31: Inhibition of PHA-induced IL-8 release in human whole blood by the monospecific anti-TNF monoclonal antibody RA14956298, and the bispecific anti-TNFα/anti-OX40L ISVD constructs F027300252, F027301104, F027301189, F027301197, and F027301199. The values correspond to the mean IC50 [nM]±SEM and represent the results from 3 different donors with triplicate measurements each.

FIG. 32: Schematic presentation of ISVD construct F027300252 showing from the N-terminus to the C-terminus the monovalent building blocks/ISVDs 1E07/1, 1CO2/1, and ALB23002, connected via 9GS linkers.

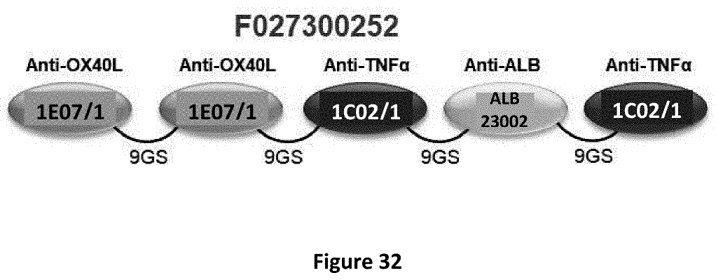

### 5 DETAILED DESCRIPTION OF THE PRESENT TECHNOLOGY

The present technology aims at providing a novel type of drug for treating autoimmune or inflammatory diseases.

The present inventors have surprisingly found that a polypeptide comprising at least four ISVDs, wherein at least two ISVDs specifically bind to TNFα, preferably human TNFα, and at least two ISVDs specifically bind to OX40L, preferably human OX40L, can be used for more efficient treatment of autoimmune or inflammatory diseases as compared to monospecific anti-TNFα or anti-OX40L polypeptides. In some embodiments, the polypeptides of the present technology are efficiently produced (e.g. in microbial hosts) and showed low viscosity at high concentrations which is advantageous and convenient for subcutaneous administration. Furthermore, such polypeptides have limited reactivity to pre-existing antibodies in the subject to be treated (i.e. antibodies present in the subject before the first treatment with the antibody construct). In preferred embodiments such polypeptides exhibit a half-life in the subject to be treated that is long enough such that consecutive treatments can be conveniently spaced apart.

The polypeptide is at least bispecific, but can also be e.g., trispecific, tetraspecific or pentaspecific. Moreover, the polypeptide is at least tetravalent, but can also be e.g. pentavalent or hexavalent, etc.

The terms “bispecific”, “trispecific”, “tetraspecific”, or “pentaspecific” all fall under the term “multispecific” and refer to binding to two, three, four or five different target molecules, respectively. The terms “bivalent”, “trivalent”, “tetravalent”, “pentavalent”, or “hexavalent” all fall under the term “multivalent” and indicate the presence of two, three, four or five binding units (such as ISVDs), respectively. For example, the polypeptide may be trispecific-pentavalent, such as a polypeptide comprising or consisting of five ISVDs, wherein two ISVDs bind to human TNFα, two ISVDs bind to human OX40L and one ISVD binds to human serum albumin (such as ISVD construct F027300252). Such a polypeptide may at the same time be biparatopic, for example if two ISVDs bind two different epitopes on human TNFα or human OX40L. The term “biparatopic” refers to binding to two different parts (e.g., epitopes) of the same target molecule.

The terms “first ISVD”, “second ISVD”, “third ISVD”, etc., as used herein only indicate the relative position of the ISVDs to each other, wherein the numbering is started from the N-terminus of the polypeptide of the present technology. The “first ISVD” is thus closer to the N-terminus than the “second ISVD”, whereas the “second ISVD” is closer to the N-terminus than the “third ISVD”, etc. Accordingly, the ISVD arrangement is inverse when considered from the C-terminus. Since the numbering is not absolute and only indicates the relative position of the at least three ISVDs it is not excluded that other binding units/building blocks such as additional ISVDs binding to TNFα or OX40L, or ISVDs binding to another target may be present in the polypeptide. Moreover, it does not exclude the possibility that other binding units/building blocks such as ISVDs can be placed in between. For instance, as described further below (see in particular, section 5.3 “(In vivo) half-life extension”), the polypeptide can further comprise another ISVD binding to human serum albumin that can even be located between e.g., the “second ISVD” and “third ISVD”.

In light of the above, the present technology provides a polypeptide comprising or consisting of at least four ISVDs, wherein at least two ISVD specifically bind to TNFα and at least two ISVDs specifically bind to OX40L, wherein the TNFα and OX40L are preferably human TNFα and human OX40L.

The components, preferably ISVDs, of the polypeptide may be linked to each other by one or more suitable linkers, such as peptidic linkers.

The use of linkers to connect two or more (poly)peptides is well known in the art. Exemplary peptidic linkers are shown in Table A-5. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO: 60) motif (for example, comprising the formula (Gly-Gly-Gly-Gly-Ser)ₙ in which n may be 1, 2, 3, 4, 5, 6, 7 or more). Some often used examples of such GS linkers are 9GS linkers (GGGGSGGGS, SEQ ID NO: 63) 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In the polypeptide of the present technology, the use of 9GS linkers to link the components of the polypeptide to each other is preferred.

In a preferred embodiment, two of the at least two ISVDs specifically binding to TNFα is positioned at the C-terminus of the polypeptide. The inventors surprisingly found that such a configuration can increase the production yield of the polypeptide.

Also, in a preferred embodiment, two of the at least two ISVDs specifically binding to OX40L are positioned at the N-terminus of the polypeptide.

Accordingly, it is preferred that the polypeptide comprises or consists of the following, in the order starting from the N-terminus of the polypeptide: a first ISVD specifically binding to OX40L, a second ISVD specifically binding to OX40L, a first ISVD specifically binding to TNFα, an optional binding unit providing the polypeptide with increased half-life as defined herein, and a second ISVD specifically binding to TNFα. The binding unit providing the polypeptide with increased half-life is preferably an ISVD.

It is even more preferred that the polypeptide comprises or consists of the following, in the order starting from the N-terminus of the polypeptide: an ISVD specifically binding to OX40L, a linker, a second ISVD specifically binding to OX40L, a linker, a first ISVD specifically binding to TNFα, a linker, an ISVD binding to human serum albumin, a linker, and a second ISVD specifically binding to TNFα, wherein each linker preferably is a 9GS linker.

Such configurations of the polypeptide can provide for increased production yield, good CMC characteristics as well as optimized functionality and stronger potency with regard to modulation of an immune response.

Preferably, the polypeptide of the present technology exhibits reduced binding by pre-existing antibodies in human serum. To this end, in one embodiment, the polypeptide comprises a valine (V) at amino acid position 11 and a leucine (L) at amino acid position 89 (according to Kabat numbering) in at least one ISVD, but preferably in each ISVD. In another embodiment, the polypeptide comprises an extension of 1 to 5 (preferably naturally occurring) amino acids, such as a single alanine (A) extension, at the C-terminus of the C-terminal ISVD. The C-terminus of an ISVD is normally VTVSS (SEQ ID NO: 125). In another embodiment the polypeptide comprises a lysine (K) or glutamine (Q) at position 110 (according to Kabat numbering) in at least one ISVD. In another embodiment, the ISVD comprises a lysine (K) or glutamine (Q) at position 112 (according to Kabat numbering) in at least one ISVD. In these embodiments, the C-terminus of the ISVD is VKVSS (SEQ ID NO: 126), VQVSS (SEQ ID NO: 127), VTVKS (SEQ ID NO:131), VTVQS (SEQ ID NO:132), VKVKS (SEQ ID NO:133), VKVQS (SEQ ID NO:134), VQVKS (SEQ ID NO:135), or VQVQS (SEQ ID NO:136) such that after addition of a single alanine the C-terminus of the polypeptide for example comprises the sequence VTVSSA (SEQ ID NO: 128), VKVSSA (SEQ ID NO: 129), VQVSSA (SEQ ID NO: 130), VTVKSA (SEQ ID NO:137), VTVQSA (SEQ ID NO:138), VKVKSA (SEQ ID NO:139), VKVQSA (SEQ ID NO:140), VQVKSA (SEQ ID NO:141), or VQVQSA (SEQ ID NO:142), preferably VKVSSA (SEQ ID NO: 129). In another embodiment, the polypeptide comprises a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD, and comprises an extension of 1 to 5 (preferably naturally occurring) amino acids, such as a single alanine (A) extension, at the C-terminus of the C-terminal ISVD (such that the C-terminus of the polypeptide for example comprises the sequence VTVSSA (SEQ ID NO: 128), VKVSSA (SEQ ID NO: 129) or VQVSSA (SEQ ID NO: 130), preferably VKVSSA (SEQ ID NO: 129)). See e.g. WO2012/175741 and WO2015/173325 for further information in this regard.

In a preferred embodiment, the polypeptide of the present technology comprises or consists of an amino acid sequence comprising a sequence identity of more than 90%, such as more than 95% or more than 99%, with SEQ ID NO: 1, wherein optionally the CDRs of the five ISVDs are as defined in items A to C (or A′ to C′ if using the Kabat definition) set forth in sections “5.1 Immunoglobulin single variable domains” and “5.3 (In vivo) half-life extension” below, respectively, wherein in particular:
the first and second ISVD specifically binding to OX40L have a CDR1 comprising the amino acid sequence of SEQ ID NO: 7, a CDR2 comprising the amino acid sequence of SEQ ID NO: 10 and a CDR3 comprising the amino acid sequence of SEQ ID NO: 13;
the third and fourth ISVD specifically binding to TNFα have a CDR1 comprising the amino acid sequence of SEQ ID NO: 8, a CDR2 comprising the amino acid sequence of SEQ ID NO: 11 and a CDR3 comprising the amino acid sequence of SEQ ID NO: 14; and
the ISVD binding to human serum albumin comprises a CDR1 comprising the amino acid sequence of SEQ ID NO: 9, a CDR2 comprising the amino acid sequence of SEQ ID NO: 12 and a CDR3 comprising the amino acid sequence of SEQ ID NO: 15,

or alternatively if using the Kabat definition:
the first and second ISVD specifically binding to OX40L have a CDR1 comprising the amino acid sequence of SEQ ID NO: 28, a CDR2 comprising the amino acid sequence of SEQ ID NO: 31 and a CDR3 comprising the amino acid sequence of SEQ ID NO: 13;
the third and fourth ISVD specifically binding to TNFα have a CDR1 comprising the amino acid sequence of SEQ ID NO: 29, a CDR2 comprising the amino acid sequence of SEQ ID NO: 32 and a CDR3 comprising the amino acid sequence of SEQ ID NO: 14; and
the ISVD binding to human serum albumin comprises a CDR1 comprising the amino acid sequence of SEQ ID NO: 30, a CDR2 comprising the amino acid sequence of SEQ ID NO: 33 and a CDR3 comprising the amino acid sequence of SEQ ID NO: 15.

Preferably, the polypeptide comprises or consists of the amino acid sequence of SEQ ID NO: 1. In a most preferred embodiment, the polypeptide consists of the amino acid sequence of SEQ ID NO: 1.

The polypeptide of the present technology preferably has at least half the binding affinity, more preferably at least the same binding affinity, to human TNFα and to human OX40L as compared to a polypeptide consisting of the amino acid of SEQ ID NO: 1 wherein the binding affinity is measured using the same method, such as Sierra SPR-32 (SPR).

### 5.1 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® (as defined herein, and including but not limited to a V_{HH}); other single variable domains, or any suitable fragment of any one thereof.

In particular, the immunoglobulin single variable domain may be a Nanobody® (such as a V_{HH}, including a humanized V_{HH} or camelized V_{H}) or a suitable fragment thereof. Nanobody®, Nanobodies® and Nanoclone® are registered trademarks.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (Reviews in Molecular Biotechnology 74: 277-302, 2001.

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences, such as Nanobodies®, has been described extensively in various publications, among which WO 94/04678, Hamers-Casterman et al. 1993 and Muyldermans et al. 2001 (Reviews in Molecular Biotechnology 74: 277-302, 2001) can be exemplified. In these methods, camelids are immunized with the target antigen in order to induce an immune response against said target antigen. The repertoire of Nanobodies obtained from said immunization is further screened for Nanobodies that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The present technology also includes fully human, humanized or chimeric sequences. For example, the present technology comprises camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996Moreover, the present technology also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g. toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized” , i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art (e.g. WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g. WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

It should be noted that one or more immunoglobulin sequences may be linked to each other and/or to other amino acid sequences (e.g. via disulphide bridges) to provide peptide constructs that may also be useful in the present technology (for example Fab′ fragments, F(ab′)2 fragments, scFv constructs, “diabodies” and other multispecific constructs). Reference is for example made to the review by Holliger and Hudson, Nat Biotechnol. 2005 Sep.; 23(9):1126-36)). Generally, when a polypeptide is intended for administration to a subject (for example for prophylactic, therapeutic and/or diagnostic purposes), it preferably comprises an immunoglobulin sequence that does not occur naturally in said subject.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

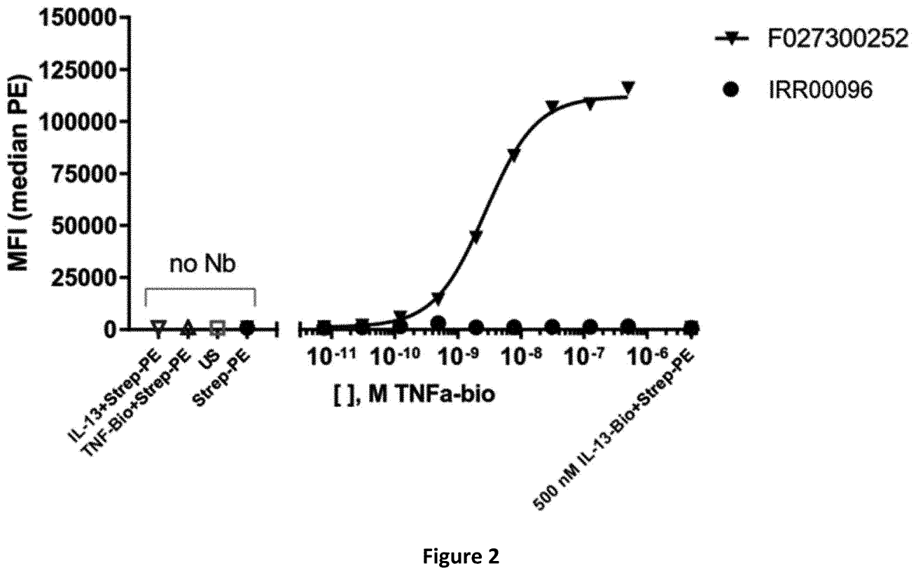

As further described in paragraph q) on pages 58 and 59 of WO 08/020079 the amino acid residues of an immunoglobulin single variable domain can be numbered according to the general numbering for VH domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the present technology may contain one or more of hallmark residues (as defined herein), such that the ISVD sequence is a Nanobody®, such as a V_{HH}, including a humanized V_{HH} or camelized V_{H}. Some preferred, but non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the present technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be a Nanobody® or a suitable fragment thereof. For a general description of Nanobodies, reference is made to the further description below, as well as to the prior art cited herein. In this respect, it should however be noted that this description and the prior art mainly described Nanobodies of the so-called “V_{H}3 class” (i.e. Nanobodies with a high degree of sequence homology to human germline sequences of the V_{H}3 class such as DP-47, DP-51 or DP-29). It should however be noted that the present technology in its broadest sense can generally use any type of Nanobody, and for example also uses the Nanobodies belonging to the so-called “V_{H}4 class” (i.e. Nanobodies with a high degree of sequence homology to human germline sequences of the V_{H}4 class such as DP-78), as for example described in WO 2007/118670.

Generally, Nanobodies (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody can be defined as an immunoglobulin sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an immunoglobulin sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an immunoglobulin sequence with the (general) structure
FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:

one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in Nanobodies

| Position | Human V_{H}3 | Hallmark Residues |
|---|---|---|
| 11 | L, V; | L, S, V, M, W, F, T, Q, E, |
| | predominantly | A, R, G, K, Y, N, P, I; |
| | L | preferably L |
| 37 | V, I, F; | F⁽¹⁾, Y, V, L, A, H, S, I, W, |
| | usually V | C, N, G, D, T, P, preferably |
| | | F⁽¹⁾ or Y |
| 44⁽⁸⁾ | G | E⁽³⁾, Q⁽³⁾, G⁽²⁾, D, A, K, R, L, |
| | | P, S, V, H, T, N, W, M, I; |
| | | preferably G⁽²⁾, E⁽³⁾ or Q⁽³⁾; |
| | | most preferably G⁽²⁾ or |
| | | Q⁽³⁾. |
| 45⁽⁸⁾ | L | L⁽²⁾, R⁽³⁾, P, H, F, G, Q, S, E, |
| | | T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, |
| | | M, R, Y, E, P, T, C, H, K, Q, |
| | | N, D; preferably W⁽²⁾, L⁽¹⁾ or F⁽¹⁾ |
| 83 | R or K; | R, K⁽⁵⁾, T, E⁽⁵⁾, Q, N, S, I, |
| | usually R | V, G, M, L, A, D, Y, H; |
| | | preferably K or R; most preferably K |
| 84 | A, T, D; | P⁽⁵⁾, S, H, L, A, V, I, T, F, D, |
| | predominantly | R, Y, N, Q, G, E; preferably |
| | A | P |
| 103 | W | W⁽⁴⁾, R⁽⁶⁾, G, S, K, A, M, Y, |
| | | L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, |
| | predominantly L | A, H; preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

The present technology inter alio uses ISVDs that can specifically bind to TNFα or OX40L. In the context of the present technology, “binding to” a certain target molecule has the usual meaning in the art as understood in the context of antibodies and their respective antigens.

The polypeptide of the present technology may comprise two or more ISVDs specifically binding to TNFα and two or more ISVDs specifically binding to OX40L. For example, the polypeptide may comprise two ISVDs that specifically bind to TNFα and two ISVDs that specifically bind to OX40L.

In some embodiments, at least one ISVD can functionally block its target molecule. For example, ISVD can block the interaction between TNFα and TNFR (TNF receptor) or can block the interaction between OX40L and OX40 (receptor) and preferably inhibit the OX40L induced release of IL2 from T-cells. Accordingly, in a preferred embodiment, the polypeptide of the present technology comprises at least two ISVDs that specifically binds to TNFα and functionally block its interaction with TNFR, and two ISVDs that specifically bind to OX40L and functionally block its interaction with OX40.

The ISVDs used in the present technology form part of a polypeptide of the present technology, which comprises or consists of at least four ISVDs, such that the polypeptide can specifically bind to TNFα and OX40L.

Accordingly, the target molecules of the at least four ISVDs as used in the polypeptide of the present technology are TNFα and OX40L. Examples are mammalian TNFα and OX40L. While human TNFα (Uniprot accession P01375) and human OX40L (Uniprot accession P23510) are preferred, the versions from other species are also amenable to the present technology, for example TNFα and IL-23 from mice, rats, rabbits, cats, dogs, goats, sheep, horses, pigs, non-human primates, such as cynomolgus monkeys (also referred to herein as “cyno”), or camelids, such as llama or alpaca.

Specific examples of ISVDs specifically binding to TNFα or OX40L that can be used in the present technology are as described in the following items A and B:

A. An ISVD that specifically binds to human OX40L and comprises
i. a CDR1 comprising the amino acid sequence of SEQ ID NO: 7 or has 2 or 1 amino acid difference(s) with SEQ ID NO: 7;
ii. a CDR2 comprising the amino acid sequence SEQ ID NO: 10 or has 2 or 1 amino acid difference(s) with SEQ ID NO: 10; and
iii. a CDR3 comprising the amino acid sequence of SEQ ID NO: 13 or has 2 or 1 amino acid difference(s) with SEQ ID NO: 13,
preferably a CDR1 comprising the amino acid sequence of SEQ ID NO: 7, a CDR2 comprising the amino acid sequence of SEQ ID NO: 10 and a CDR3 comprising the amino acid sequence of SEQ ID NO: 13.

Preferred examples of such an ISVD that specifically binds to human OX40L have one or more (and preferably all) framework regions as indicated for construct 1E07/1 in Table A-2 (in addition to the CDRs as defined in the preceding item A), and most preferred is an ISVD comprising the full amino acid sequence of construct 1E07/1 (SEQ ID NOs: 2 or 3, see Table A-1 and A-2).

Also in a preferred embodiment, the amino acid sequence of the ISVD specifically binding to human OX40L may have a sequence identity of more than 90%, such as more than 95% or more than 99%, with SEQ ID NO: 2 or 3, wherein optionally the CDRs are as defined in the preceding item A. In particular, the ISVD specifically binding to OX40L preferably comprises the amino acid sequence of SEQ ID NO: 2 or 3.

When such an ISVD specifically binding to OX40L has 2 or 1 amino acid difference in at least one CDR relative to a corresponding reference CDR sequence (item A above), the ISVD preferably has at least half the binding affinity, more preferably at least the same binding affinity to human OX40L as the construct 1E07/1 set forth in SEQ ID NO: 2 or 3, wherein the binding affinity is measured using the same method, such as SPR.

B. An ISVD that specifically binds to human TNFα and comprises
i. a CDR1 comprising the amino acid sequence SEQ ID NO: 8 or has 2 or 1 amino acid difference(s) with SEQ ID NO: 8;
ii. a CDR2 comprising the amino acid sequence SEQ ID NO: 11 or has 2 or 1 amino acid difference(s) with SEQ ID NO: 11; and
iii. a CDR3 comprising the amino acid sequence SEQ ID NO: 14 or has 2 or 1 amino acid difference(s) with SEQ ID NO: 14,
preferably a CDR1 comprising the amino acid sequence of SEQ ID NO: 8, a CDR2 comprising the amino acid sequence of SEQ ID NO: 11 and a CDR3 comprising the amino acid sequence of SEQ ID NO: 14.

Preferred examples of such an ISVD that specifically binds to human TNFα have one or more (and preferably all) framework regions as indicated for construct 1C02/1 in Table A-2 (in addition to the CDRs as defined in the preceding item B), and most preferred is an ISVD comprising the full amino acid sequence of construct 1C02/1 (SEQ ID NOs: 4 or 6, see Table A-1 and A-2).

Also in a preferred embodiment, the amino acid sequence of an ISVD specifically binding to human TNFα may have a sequence identity of more than 90%, such as more than 95% or more than 99%, with SEQ ID NO: 4 or 6, wherein optionally the CDRs are as defined in the preceding item B. In particular, the ISVD specifically binding to human TNFα preferably comprises the amino acid sequence of SEQ ID NOs: 4 or 6.

When such an ISVD specifically binding to human TNFα has 2 or 1 amino acid difference in at least one CDR relative to a corresponding reference CDR sequence (item B above), the ISVD preferably has at least half the binding affinity, more preferably at least the same binding affinity to human TNFα as construct 1C02/1 set forth in SEQ ID NO: 4 or 6, wherein the binding affinity is measured using the same method, such as SPR.

Preferably, each of the ISVDs as defined under items A and B above is comprised in the polypeptide of the present technology.

Such a polypeptide of the present technology comprising each of the ISVDs as defined under items A and B above preferably has at least half the binding affinity, more preferably at least the same binding affinity, to human OX40L and to human TNFα as a polypeptide consisting of the amino acid of SEQ ID NO: 1, wherein the binding affinity is measured using the same method, such as SPR.

The SEQ ID NOs referred to in the above items A and B are based on the CDR definition according to the AbM definition (see Table A-2). It is noted that the SEQ ID NOs defining the same CDRs according to the Kabat definition (see Table A-2.1) can likewise be used in the above items A and B.

Accordingly, the specific examples of ISVDs specifically binding to TNFα or OX40L that can be used in the present technology are as described above using the AbM definition can be also described using the Kabat definition as set forth in items A′ to B′ below:

A′. An ISVD that specifically binds to human OX40L and comprises
i. a CDR1 comprising the amino acid sequence of SEQ ID NO: 28 or has 2 or 1 amino acid difference(s) with SEQ ID NO: 28;
ii. a CDR2 comprising the amino acid sequence SEQ ID NO: 31 or has 2 or 1 amino acid difference(s) with SEQ ID NO: 31; and
iii. a CDR3 comprising the amino acid sequence of SEQ ID NO: 13 or has 2 or 1 amino acid difference(s) with SEQ ID NO: 13,
preferably a CDR1 comprising the amino acid sequence of SEQ ID NO: 28, a CDR2 comprising the amino acid sequence of SEQ ID NO: 31 and a CDR3 comprising the amino acid sequence of SEQ ID NO: 13.

Preferred examples of such an ISVD that specifically binds to human OX40L have one or more (and preferably all) framework regions as indicated for construct 1E07/1 in Table A-2-1 (in addition to the CDRs as defined in the preceding item A′), and most preferred is an ISVD comprising the full amino acid sequence of construct 1E07/1 (SEQ ID NOs: 2 or 3, see Table A-1 and A-2-1).

B′. An ISVD that specifically binds to human TNFα and comprises
i. a CDR1 comprising the amino acid sequence SEQ ID NO: 29 or has 2 or 1 amino acid difference(s) with SEQ ID NO: 29;
ii. a CDR2 comprising the amino acid sequence SEQ ID NO: 32 or has 2 or 1 amino acid difference(s) with SEQ ID NO: 32; and
iii. a CDR3 comprising the amino acid sequence SEQ ID NO: 14 or has 2 or 1 amino acid difference(s) with SEQ ID NO: 14,
preferably a CDR1 comprising the amino acid sequence of SEQ ID NO: 29, a CDR2 comprising the amino acid sequence of SEQ ID NO: 32 and a CDR3 comprising the amino acid sequence of SEQ ID NO: 14.

Preferred examples of such an ISVD that specifically binds to human TNFα have one or more (and preferably all) framework regions as indicated for construct 1C02/1 in Table A-2-1 (in addition to the CDRs as defined in the preceding item B′), and most preferred is an ISVD comprising the full amino acid sequence of construct 1C02/1 (SEQ ID NOs: 4 or 6, see Table A-1 and A-2-1).

The percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position).

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

An “amino acid difference” as used herein refers to a deletion, insertion or substitution of a single amino acid residue vis-à-vis a reference sequence, and preferably is a substitution.

Amino acid substitutions are preferably conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

### 5.2 Specificity

The terms “specificity”, “binding specifically” or “specific binding” refer to the number of different target molecules, such as antigens, from the same organism to which a particular binding unit, such as an ISVD, can bind with sufficiently high affinity (see below).

“Specificity”, “binding specifically” or “specific binding” are used interchangeably herein with “selectivity”, “binding selectively” or “selective binding”. Binding units, such as ISVDs, preferably specifically bind to their designated targets.

The specificity/selectivity of a binding unit can be determined based on affinity. The affinity denotes the strength or stability of a molecular interaction. The affinity is commonly given as by the KD, or dissociation constant, comprising units of mol/liter (or M). The affinity can also be expressed as an association constant, KA, which equals 1/KD and has units of (mol/liter)⁻¹ (or M⁻¹).

The affinity is a measure for the binding strength between a moiety and a binding site on the target molecule: the lesser the value of the KD, the stronger the binding strength between a target molecule and a targeting moiety.

Typically, binding units used in the present technology (such as ISVDs) will bind to their targets with a dissociation constant (KD) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (KA) of 10⁵ to 10¹² liter/moles or more, and preferably 10⁷ to 10¹² liter/moles or more and more preferably 10⁸ to 10¹² liter/moles).

Any KD value greater than 10⁻⁴ mol/liter (or any KA value lower than 10⁴ liters/mol) is generally considered to indicate non-specific binding.

The KD for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less.

Accordingly, specific/selective binding may mean that—using the same measurement method, e.g. SPR—a binding unit (or polypeptide comprising the same) binds to TNFα and/or OX40L with a KD value of 10⁻⁵ to 10⁻¹² moles/liter or less and binds to related cytokines with a KD value greater than 10⁻⁴ moles/liter. Examples of OX40L related targets are human TRAIL, CD30L, CD40L and RANKL. Examples of related cytokines for TNFα are TNF superfamily members FASL, TNFβ, LIGHT, TL-1A, RANKL. Thus, in an embodiment of the present technology, at least two ISVDs comprised in the polypeptide binds to TNFα with a KD value of 10⁻⁵ to 10⁻¹² moles/liter or less and binds to FASL, TNFβ, LIGHT, TL-1A, RANKL of the same species with a KD value greater than 10⁻⁴ moles/liter, and at least two ISVDs comprised in the polypeptide bind to OX40L with a KD value of 10⁻⁵ to 10⁻¹² moles/liter or less and binds to human TRAIL, CD30L, CD40L and RANKL of the same species with a KD value greater than 10⁻⁴ moles/liter.

Thus, the polypeptide of the present technology preferably has at least half the binding affinity, more preferably at least the same binding affinity, to human TNFα and to human OX40L as compared to a polypeptide consisting of the amino acid of SEQ ID NO: 1, wherein the binding affinity is measured using the same method, such as SPR.

Specific binding to a certain target from a certain species does not exclude that the binding unit can also specifically bind to the analogous target from a different species. For example, specific binding to human TNFα does not exclude that the binding unit (or a polypeptide comprising the same) can also specifically bind to TNFα from cynomolgus monkeys. Likewise, for example, specific binding to human OX40L does not exclude that the binding unit (or a polypeptide comprising the same) can also specifically bind to OX40L from cynomolgus monkeys (“cyno”).

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (KA), by means of the relationship [KD=1/KA].

The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BlAcore® system (BlAcore International AB, a GE Healthcare company, Uppsala, Sweden and Piscataway, N.J.). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al. (1991, Anal. Biochem. 198: 268-277).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

### 5.3 (In Vivo) Half-Life Extension

The polypeptide may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t1/2beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG, and preferably is a binding unit that can bind to human serum albumin. The binding unit is preferably an ISVD.

For example, WO 04/041865 describes Nanobodies® binding to serum albumin (and in particular against human serum albumin) that can be linked to other proteins (such as one or more other Nanobodies binding to a desired target) in order to increase the half-life of said protein.

The international application WO 06/122787 describes a number of Nanobodies® against (human) serum albumin. These Nanobodies® include the Nanobody® called Alb-1 (SEQ ID NO: 52 in WO 06/122787) and humanized variants thereof, such as Alb-8 (SEQ ID NO: 62 in WO 06/122787). Again, these can be used to extend the half-life of therapeutic proteins and polypeptide and other therapeutic entities or moieties.

Moreover, WO2012/175400 describes a further improved version of Alb-1, called Alb-23.

In a preferred embodiment, the polypeptide comprises a serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown on pages 7-9 of WO2012/175400 and the albumin binders described in WO2012/175741, WO2015/173325, WO2017/080850, WO2017/085172, WO2018/104444, WO2018/134235, WO2018/134234. Some preferred serum albumin binders are also shown in Table A-4. A particularly preferred further component of the polypeptide of the present technology is as described in item C:

C. An ISVD that binds to human serum albumin and comprises
i. a CDR1 comprising the amino acid sequence of SEQ ID NO: 9 or has 2 or 1 amino acid difference(s) with SEQ ID NO: 9;
ii. a CDR2 comprising the amino acid sequence of SEQ ID NO: 12 or has 2 or 1 amino acid difference(s) with SEQ ID NO: 12; and
iii. a CDR3 comprising the amino acid sequence of SEQ ID NO: 15 or has 2 or 1 amino acid difference(s) with SEQ ID NO: 15;
preferably a CDR1 comprising the amino acid sequence of SEQ ID NO:9, a CDR2 comprising the amino acid sequence of SEQ ID NO: 12 and a CDR3 comprising the amino acid sequence of SEQ ID NO: 15.

Preferred examples of such an ISVD that binds to human serum albumin have one or more (and preferably all) framework regions as indicated for construct ALB23002 in Table A-2 (in addition to the CDRs as defined in the preceding item C), and most preferred is an ISVD comprising the full amino acid sequence of construct ALB23002 (SEQ ID NO: 5, see Table A-1 and A-2).

Item C can be also described using the Kabat definition as:

C′. An ISVD that binds to human serum albumin and comprises
i. a CDR1 comprising the amino acid sequence of SEQ ID NO: 30 or has 2 or 1 amino acid difference with SEQ ID NO: 30;
ii. a CDR2 comprising the amino acid sequence of SEQ ID NO: 33 or has 2 or 1 amino acid difference with SEQ ID NO: 33; and
iii. a CDR3 comprising the amino acid sequence of SEQ ID NO: 15 or has 2 or 1 amino acid difference with SEQ ID NO: 15;
preferably a CDR1 comprising the amino acid sequence of SEQ ID NO: 30, a CDR2 comprising the amino acid sequence of SEQ ID NO: 33 and a CDR3 comprising the amino acid sequence of SEQ ID NO: 15.

Preferred examples of such an ISVD that binds to human serum albumin have one or more, and preferably all, framework regions as indicated for construct ALB23002 in Table A-2.1 (in addition to the CDRs as defined in the preceding item C′), and most preferred is an ISVD comprising the full amino acid sequence of construct ALB23002 (SEQ ID NO: 5, see Table A-1 and A-2.1).

Also in a preferred embodiment, the amino acid sequence of an ISVD binding to human serum albumin may have a sequence identity of more than 90%, such as more than 95% or more than 99%, with SEQ ID NO: 5, wherein optionally the CDRs are as defined in the preceding item C. In particular, the ISVD binding to human serum albumin preferably comprises the amino acid sequence of SEQ ID NO: 5.

When such an ISVD binding to human serum albumin has 2 or 1 amino acid difference in at least one CDR relative to a corresponding reference CDR sequence (item C above), the ISVD has at least half the binding affinity, preferably at least the same binding affinity to human serum albumin as construct ALB23002 set forth in SEQ ID NO: 5, wherein the binding affinity is measured using the same method, such as SPR.

When such an ISVD binding to human serum albumin comprises a C-terminal position it exhibits a C-terminal alanine (A) or glycine (G) extension and is preferably selected from SEQ ID NOs: 46, 47, 49, 51, 52, 53, 54, 55, 56, and 58 (see table A-4 below). In a preferred embodiment, the ISVD binding to human serum albumin comprises another position than the C-terminal position (i.e. is not the C-terminal ISVD of the polypeptide of the present technology) and is selected from SEQ ID NOs: 5, 44, 45, 48, and 50 (see table A-4 below).

### 5.4 Nucleic Acid Molecules

Also provided is a nucleic acid molecule encoding the polypeptide of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the polypeptide of the present technology is also encompassed by the present technology.

A nucleic acid may be for example DNA, RNA, or a hybrid thereof, and may also comprise (e.g. chemically) modified nucleotides, like PNA. It can be single- or double-stranded, and is preferably in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly)peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.5 Vectors

Also provided is a vector comprising the nucleic acid molecule encoding the polypeptide of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector preferably is an expression vector, i.e. a vector suitable for expressing an encoded polypeptide or construct under suitable conditions, e.g. when the vector is introduced into a (e.g. human) cell. For DNA-based vectors, this usually includes the presence of elements for transcription (e.g. a promoter and a polyA signal) and translation (e.g. Kozak sequence).

Preferably, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### 5.6 Compositions

The present technology also provides a composition comprising at least one polypeptide of the present technology, at least one nucleic acid molecule encoding a polypeptide of the present technology or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### 5.7 Host Organisms

The present technology also pertains to host cells or host organisms comprising the polypeptide of the present technology, the nucleic acid encoding the polypeptide of the present technology, and/or the vector comprising the nucleic acid molecule encoding the polypeptide of the present technology.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

### 5.8 Methods and uses of the Polypeptide

The present technology also provides a method for producing the polypeptide of the present technology. The method may comprise transforming/transfecting a host cell or host organism with a nucleic acid encoding the polypeptide, expressing the polypeptide in the host, optionally followed by one or more isolation and/or purification steps. Specifically, the method may comprise:

a) expressing, in a suitable host cell or host organism or in another suitable expression system, a nucleic acid sequence encoding the polypeptide; optionally followed by:

b) isolating and/or purifying the polypeptide.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

The polypeptide of the present technology, a nucleic acid molecule or vector as described, or a composition comprising the polypeptide of the present technology, nucleic acid molecule or vector—preferably the polypeptide or a composition comprising the same—are useful as a medicament.

Accordingly, the present technology provides the polypeptide of the present technology, a nucleic acid molecule or vector as described, or a composition comprising the polypeptide of the present technology, nucleic acid molecule or vector for use as a medicament.

Also provided is the polypeptide of the present technology, a nucleic acid molecule or vector as described, or a composition comprising the polypeptide of the present technology, nucleic acid molecule or vector for use in the (prophylactic or therapeutic) treatment of an autoimmune or an inflammatory disease.

Further provided is a (prophylactic and/or therapeutic) method of treating an autoimmune disease or an inflammatory disease, wherein said method comprises administering, to a subject in need thereof, a pharmaceutically active amount of the polypeptide of the present technology, a nucleic acid molecule or vector as described, or a composition comprising the polypeptide of the present technology, nucleic acid molecule or vector.

Further provided is the use of the polypeptide of the present technology, a nucleic acid molecule or vector as described, or a composition comprising the polypeptide of the present technology, nucleic acid molecule or vector in the preparation of a pharmaceutical composition, preferably for treating an autoimmune disease or an inflammatory disease.

The autoimmune or inflammatory disease may for example be rheumatoid arthritis; inflammatory bowel disease, such as Crohn's disease and ulcerative colitis; psoriasis, Hidradenitis suppurativa; and graft-versus-host-disease.

A “subject” as referred to in the context of the present technology can be any animal, preferably a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal, but preferably is a human subject.

Substances (including polypeptides, nucleic acid molecules and vectors) or compositions may be administered to a subject by any suitable route of administration, for example by enteral (such as oral or rectal) or parenteral (such as epicutaneous, sublingual, buccal, nasal, intra-articular, intradermal, intramuscular, intraperitoneal, intravenous, subcutaneous, transdermal, or transmucosal) administration. Parenteral administration, such as intramuscular, subcutaneous or intradermal, administration is preferred. Most preferred is subcutaneous administration.

An effective amount of a polypeptide, a nucleic acid molecule or vector as described, or a composition comprising the polypeptide, nucleic acid molecule or vector can be administered to a subject in order to provide the intended treatment results.

One or more doses can be administered. If more than one dose is administered, the doses can be administered in suitable intervals in order to maximize the effect of the polypeptide, composition, nucleic acid molecule or vector.

TABLE A-1
Amino acid sequences of the different
monovalent VHH building blocks identified
within the pentavalent polypeptide F027300252
(“ID” refers to the SEQ ID NO as used herein)

| | | Amino acid |
|---|---|---|
| Name | ID | sequence |
| 1E07/1 | 2 | DVQLVESGGGWQPGGS |
| (building | | LRLSCAASGRTFSSIY |
| block 1) | | AKGWFRQAPGKEREFV |
| | | AAISRSGRSTSYADSV |
| | | KGRFTISRDNSKNTVY |
| | | LQMNSLRPEDTALYYC |
| | | AAVGGATTVTASEWDY |
| | | WGQGTLVTVSS |
| 1E07/1 | 3 | EVQLVESGGGWQPGGS |
| (building | | LRLSCAASGRTFSSIY |
| block 2) | | AKGWFRQAPGKEREFV |
| | | AAISRSGRSTSYADSV |
| | | KGRFTISRDNSKNTVY |
| | | LQMNSLRPEDTALYYC |
| | | AAVGGATTVTASEWDY |
| | | WGQGTLVTVSS |
| 1C02/1 | 4 | EVQLVESGGGWQPGGS |
| (building | | LRLSCAASGFTFSDYW |
| block 3) | | MYWVRQAPGKGLEWVS |
| | | EINTNGLITKYPDSVK |
| | | GRFTISRDNAKNTLYL |
| | | QMNSLRPEDTALYYCA |
| | | RSPSGFNRGQGTLVTV |
| | | SS |
| ALB23002 | 5 | EVQLVESGGGVVQPGG |
| (building | | SLRLSCAASGFTFRSF |
| block 4) | | GMSWVRQAPGKGPEWV |
| | | SSISGSGSDTLYADSV |
| | | KGRFTISRDNSKNTLY |
| | | LQMNSLRPEDTALYYC |
| | | TIGGSLSRSSQGTLVT |
| | | VSS |
| 1C02/1 | 6 | EVQLVESGGGVVQPGG |
| (building | | SLRLSCAASGFTFSDY |
| block 5) | | WMYWVRQAPGKGLEWV |
| | | SEINTNGLITKYPDSV |
| | | KGRFTISRDNAKNTLY |
| | | LQMNSLRPEDTALYYC |
| | | ARSPSGFNRGQGTLVK |
| | | VSS |

TABLE A-2 Sequences for CDRs according to AbM
numbering and frameworks (“ID” refers to the
given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 2 | 1E07/ | 16 | DVQLV | 7 | GRTF | 18 | WFRQ | 10 | AISR | 21 | YADSVK | 13 | VGGA | 24 | WGQG |
| | 1_BB1 | | ESGGG | | SSIY | | APGK | | SGRS | | GRFTIS | | TTVT | | TLVT |
| | | | VVQPG | | AKG | | FRFF | | TS | | RDNSKN | | ASFW | | VSS |
| | | | GSIRL | | | | VA | | | | TVYIQM | | DY | | |
| | | | SCAAS | | | | | | | | NSIRPF | | | | |
| | | | | | | | | | | | DTAIYY | | | | |
| | | | | | | | | | | | CAA | | | | |
| 3 | 1E07/ | 17 | EVQLV | 7 | GRTF | 18 | WFRQ | 10 | AISR | 21 | YADSVK | 13 | VGGA | 24 | WGQG |
| | 1_BB2 | | ESGGG | | SSIY | | APGK | | SGRS | | GRFTIS | | TTVT | | TLVT |
| | | | VVQPG | | AKG | | EREF | | TS | | RDNSKN | | ASEW | | VSS |
| | | | GSLRL | | | | VA | | | | TVYLQM | | DY | | |
| | | | SCAAS | | | | | | | | NSLRPE | | | | |
| | | | | | | | | | | | DTALYY | | | | |
| | | | | | | | | | | | CAA | | | | |
| 4 | 1C02/ | 17 | EVQLV | 8 | GFTF | 19 | WVRQ | 11 | EINT | 22 | YPDSVK | 14 | SPSG | 25 | RGQG |
| | 1_BB3 | | ESGGG | | SDY | | APGK | | NGLI | | GRFTIS | | FN | | TLVT |
| | | | VVQPG | | WMY | | GLEW | | TK | | RDNAKN | | | | VSS |
| | | | GSLRL | | | | VS | | | | TLYLQM | | | | |
| | | | SCAAS | | | | | | | | NSLRPE | | | | |
| | | | | | | | | | | | DTALYY | | | | |
| | | | | | | | | | | | CAR | | | | |
| 5 | ALB2 | 17 | EVQLV | 9 | GFTF | 20 | WVRQ | 12 | SISG | 23 | YADSVK | 15 | GGSL | 26 | SSQG |
| | 3002 | | ESGGG | | RSF | | APGK | | SGSD | | GRFTIS | | SR | | TLVT |
| | | | VVQPG | | GMS | | GPEW | | TL | | RDNSKN | | | | VSS |
| | | | GSLRL | | | | VS | | | | TLYLQM | | | | |
| | | | SCAAS | | | | | | | | NSLRPE | | | | |
| | | | | | | | | | | | DTALYY | | | | |
| | | | | | | | | | | | CT | | | | |
| 6 | 1C02/ | 17 | EVQLV | 8 | GFTF | 19 | WVRQ | 11 | EINT | 22 | YPDSVK | 14 | SPSG | 27 | RGQG |
| | 1_BB5 | | ESGGG | | SDY | | APGK | | NGLI | | GRFTIS | | FN | | TLVK |
| | | | VVQPG | | WMY | | GLEW | | TK | | RDNAKN | | | | VSS |
| | | | GSLRL | | | | VS | | | | TLYLQM | | | | |
| | | | SCAAS | | | | | | | | NSLRPE | | | | |
| | | | | | | | | | | | DTALYY | | | | |
| | | | | | | | | | | | CAR | | | | |

TABLE A-2.1
Sequences for CDRs according to
Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 2 | 1E07/ | 34 | DVQLV | 28 | SIY | 38 | WFRQ | 31 | AISR | 41 | RFTIS | 13 | VGGA | 24 | WGQ |
| | 1_BB1 | | ESGGG | | AKG | | APGK | | SGR | | RDNSK | | TTVT | | GTL |
| | | | VVQPG | | | | EREF | | STSY | | NTVYL | | ASEW | | VTV |
| | | | GSLRL | | | | VA | | ADS | | QMNSL | | DY | | SS |
| | | | SCAAS | | | | | | VKG | | RPEDT | | | | |
| | | | GRTFS | | | | | | | | ALYYC | | | | |
| | | | | | | | | | | | AA | | | | |
| 3 | 1E07/ | 35 | EVQLV | 28 | SIY | 38 | WFRQ | 31 | AISR | 41 | RFTIS | 13 | VGGA | 24 | WGQ |
| | 1_BB2 | | ESGGG | | AKG | | APGK | | SGR | | RDNSK | | TTVT | | GTL |
| | | | VVQPG | | | | EREF | | STSY | | NTVYL | | ASEW | | VTV |
| | | | GSLRL | | | | VA | | ADS | | QMNSL | | DY | | SS |
| | | | SCAAS | | | | | | VKG | | RPEDT | | | | |
| | | | GRTFS | | | | | | | | ALYYC | | | | |
| | | | | | | | | | | | AA | | | | |
| 4 | 1C02/ | 36 | EVQLV | 29 | DYW | 39 | WVRQ | 32 | EINT | 42 | RFTIS | 14 | SPS | 25 | RGQ |
| | 1_BB3 | | ESGGG | | MY | | APGK | | NGL | | RDNAK | | GFN | | GTL |
| | | | VVQPG | | | | GLEW | | ITKY | | NTLYL | | | | VTV |
| | | | GSLRL | | | | VS | | PDS | | QMNSL | | | | SS |
| | | | SCAAS | | | | | | VKG | | RPEDT | | | | |
| | | | GFTFS | | | | | | | | ALYYC | | | | |
| | | | | | | | | | | | AR | | | | |
| 5 | ALB2 | 37 | EVQLV | 30 | SFG | 40 | WVRQ | 33 | SISG | 43 | RFTIS | 15 | GGS | 26 | SSQ |
| | 3002 | | ESGGG | | MS | | APGK | | SGS | | RDNSK | | LSR | | GTL |
| | | | VVQPG | | | | GPEW | | DTLY | | NTLYL | | | | VTV |
| | | | GSLRL | | | | VS | | ADS | | QMNSL | | | | SS |
| | | | SCAAS | | | | | | VKG | | RPEDT | | | | |
| | | | GFTFR | | | | | | | | ALYYC | | | | |
| | | | | | | | | | | | TI | | | | |
| 6 | 1C02/ | 36 | EVQLV | 29 | DYW | 39 | WVRQ | 32 | EINT | 42 | RFTIS | 14 | SPS | 27 | RGQ |
| | 1_BB5 | | ESGGG | | MY | | APGK | | NGL | | RDNAK | | GFN | | GTL |
| | | | VVQPG | | | | GLEW | | ITKY | | NTLYL | | | | VKV |
| | | | GSLRL | | | | VS | | PDS | | QMNSL | | | | SS |
| | | | SCAAS | | | | | | VKG | | RPEDT | | | | |
| | | | GFTFS | | | | | | | | ALYYC | | | | |
| | | | | | | | | | | | AR | | | | |

TABLE A-3
Amino acid sequences of selected
multivalent polypeptide
(“ID” refers to the given SEQ ID NO)

| Name | ID | Amino acid sequence |
|---|---|---|
| F027300252 | 1 | DVQLVESGGGVVQPGGSLRLSCAASGRTFSSIYA |
| | | KGWFRQAPGKEREFVAAISRSGRSTSYADSVKGR |
| | | FTISRDNSKNTVYLQMNSLRPEDTALYYCAAVGG |
| | | ATTVTASEWDYWGQGTLVTVSSGGGGSGGGSEVQ |
| | | LVESGGGVVQPGGSLRLSCAASGRTFSSIYAKGW |
| | | FRQAPGKEREFVAAISRSGRSTSYADSVKGRFTI |
| | | SRDNSKNTVYLQMNSLRPEDTALYYCAAVGGATT |
| | | VTASEWDYWGQGTLVTVSSGGGGSGGGSEVQLVE |
| | | SGGGVVQPGGSLRLSCAASGFTFSDYWMYWVRQA |
| | | PGKGLEWVSEINTNGLITKYPDSVKGRFTISRDN |
| | | AKNTLYLQMNSLRPEDTALYYCARSPSGFNRGQG |
| | | TLVTVSSGGGGSGGGSEVQLVESGGGVVQPGGSL |
| | | RLSCAASGFTFRSFGMSWVRQAPGKGPEWVSSIS |
| | | GSGSDTLYADSVKGRFTISRDNSKNTLYLQMNSL |
| | | RPEDTALYYCTIGGSLSRSSQGTLVTVSSGGGGS |
| | | GGGSEVQLVESGGGVVQPGGSLRLSCAASGFTFS |
| | | DYWMYWVRQAPGKGLEWVSEINTNGLITKYPDSV |
| | | KGRFTISRDNAKNTLYLQMNSLRPEDTALYYCAR |
| | | SPSGFNRGQGTLVKVSSA |

TABLE A-4
Serum albumin binding ISVD
sequences (“ID” refers to the
SEQ ID NO as used herein)

| Name | ID | Amino acid sequence |
|---|---|---|
| Alb8 | 44 | EVQLVESGGGLVQPGNSLRLSCAA |
| | | SGFTFSSFGMSWVRQAPGKGLEWV |
| | | SSISGSGSDTLYADSVKGRFTISR |
| | | DNAKTTLYLQMNSLRPEDTAVYYC |
| | | TIGGSLSRSSQGTLVTVSS |
| Alb23 | 45 | EVQLLESGGGLVQPGGSLRLSCAA |
| | | SGFTFRSFGMSWVRQAPGKGPEWV |
| | | SSISGSGSDTLYADSVKGRFTISR |
| | | DNSKNTLYLQMNSLRPEDTAVYYC |
| | | TIGGSLSRSSQGTLVTVSS |
| Alb129 | 46 | EVQLVESGGGVVQPGNSLRLSCAA |
| | | SGFTFSSFGMSWVRQAPGKGLEWV |
| | | SSISGSGSDTLYADSVKGRFTISR |
| | | DNAKTTLYLQMNSLRPEDTATYYC |
| | | TIGGSLSRSSQGTLVTVSSA |
| Alb132 | 47 | EVQLVESGGGVVQPGGSLRLSCAA |
| | | SGFTFRSFGMSWVRQAPGKGPEWV |
| | | SSISGSGSDTLYADSVKGRFTISR |
| | | DNSKNTLYLQMNSLRPEDTATYYC |
| | | TIGGSLSRSSQGTLVTVSSA |
| Alb11 | 48 | EVQLVESGGGLVQPGNSLRLSCAA |
| | | SGFTFSSFGMSWVRQAPGKGLEWV |
| | | SSISGSGSDTLYADSVKGRFTISR |
| | | DNAKTTLYLQMNSLRPEDTAVYYC |
| | | TIGGSLSRSSQGTLVTVSS |
| Alb11 | 49 | EVQLVESGGGLVQPGNSLRLSCAA |
| (S112K)- | | SGFTFSSFGMSWVRQAPGKGLEWV |
| A | | SSISGSGSDTLYADSVKGRFTISR |
| | | DNAKTTLYLQMNSLRPEDTAVYYC |
| | | TIGGSLSRSSQGTLVKVSSA |
| Alb82 | 50 | EVQLVESGGGVVQPGNSLRLSCAA |
| | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSS |
| Alb82- | 51 | EVQLVESGGGVVQPGNSLRLSCAA |
| A | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSSA |
| Alb82- | 52 | EVQLVESGGGWQPGNSLRLSCAAS |
| AA | | GFTFSSFGMSWVRQAPGKGLEWVS |
| | | SISGSGSDTLYADSVKGRFTISRD |
| | | NAKTTLYLQMNSLRPEDTALYYCT |
| | | IGGSLSRSSQGTLVTVSSAA |
| Alb82- | 53 | EVQLVESGGGVVQPGNSLRLSCAA |
| AAA | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSSAAA |
| Alb82- | 54 | EVQLVESGGGVVQPGNSLRLSCAA |
| G | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSSG |
| Alb82- | 55 | EVQLVESGGGVVQPGNSLRLSCAA |
| GG | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSSGG |
| Alb82- | 56 | EVQLVESGGGVVQPGNSLRLSCAA |
| GGG | | SGFTFSSFGMSWVRQAPGKGLEWV |
| | | SSISGSGSDTLYADSVKGRFTISR |
| | | DNAKTTLYLQMNSLRPEDTALYYC |
| | | TIGGSLSRSSQGTLVTVSSGGG |
| Alb2 | 5 | EVQLVESGGGVVQPGGSLRLSCAA |
| 3002 | | SGFTFRSFGMSWVRQAPGKGPEWV |
| | | SSISGSGSDTLYADSVKGRFTISR |
| | | DNSKNTLYLQMNSLRPEDTALYYC |
| | | TIGGSLSRSSQGTLVTVSS |
| Alb223 | 58 | EVQLVESGGGVVQPGGSLRLSCAA |
| | | SGFTFRSFGMSWVRQAPGKGPEWV |
| | | SSISGSGSDTLYADSVKGRFTISR |
| | | DNSKNTLYLQMNSLRPEDTALYYC |
| | | TIGGSLSRSSQGTLVTVSSA |

TABLE A-5
Linker sequences (“ID” refers
to the SEQ ID NO as used herein)

| | | Amino acid |
|---|---|---|
| Name | ID | sequence |
| 3A linker | 59 | AAA |
| 5GS linker | 60 | GGGGS |
| 7GS linker | 61 | SGGSGGS |
| 8GS linker | 62 | GGGGSGGS |
| 9GS linker | 63 | GGGGSGGGS |
| 10GS linker | 64 | GGGGSGGGGS |
| 15GS linker | 65 | GGGGSGGGGSGGGGS |
| 18GS linker | 66 | GGGGSGGGGSGGGGSGGS |
| 20GS linker | 67 | GGGGSGGGGSGGGGSGGGGS |
| 25GS linker | 68 | GGGGSGGGGSGGGGSGGGGSG |
| | | GGGS |
| 30GS linker | 69 | GGGGSGGGGSGGGGSGGGGSG |
| | | GGGSGGGGS |
| 35GS linker | 70 | GGGGSGGGGSGGGGSGGGGSG |
| | | GGGSGGGGSGGGGS |
| 40GS linker | 71 | GGGGSGGGGSGGGGSGGGGS |
| | | GGGGSGGGGSGGGGSGGGGS |
| G1 hinge | 72 | EPKSCDKTHTCPPCP |
| 9GS-G1 hinge | 73 | GGGGSGGGSEPKSCDKTHTCP |
| Llama upper | 74 | PCPEPKTPKPQPAAA |
| long hinge | | |
| region | | |
| G3 hinge | 75 | ELKTPLGDTTHTCPRCPEPKS |
| | | CDTPPPCPRCPEPKSCDTPPP |
| | | CPRCPEPKSCDTPPPCPRCP |

### 6 EXAMPLES

### 6.1 Example 1: Multispecific ISVD Construct Generation

Identification of ISVD-containing polypeptide F027300252 (SEQ ID NO: 1) binding to TNFα and OX40L resulted from a data-driven multispecific engineering and formatting campaign in which anti-TNFα V_{HH} building blocks (TNF06C11 (WO2017081320), TNF01C02 (WO2015173325, SEQ ID NO: 327), and VHH #3 (WO2004041862)), anti-OX40L V_{HH} building blocks (OX40L1E07, OX40L1B11, and OX40L15B07, see WO2011073180)) and anti-HSA V_{HH} building block ALB23002 (see WO2017134234, SEQ ID NO:10/WO2018131234) were included. Different positions/orientations of the building blocks and different linker lengths (9GS, 20GS vs 35GS) were applied and proved to be critical for different parameters (potency, cross-reactivity, expression, etc.). Potency in this context refers to the inhibition of TNFα-induced NFκB activation and inhibition of OX40L induced co-stimulation of T cells in vitro as assayed in Examples 7 and 9.

A panel comprising 84 constructs (Table 2) was transformed in Pichia pastoris for small scale productions. Induction of ISVD construct expression occurred by stepwise addition of methanol. Clarified medium with secreted ISVD construct was used as starting material for purification via Protein A affinity chromatography followed by desalting. The purified samples were used for functional characterisation and expression evaluation.

TABLE 2
Listing of the 84 different multispecific ISVD formats evaluated. BB = building block, ALB =
ALB23002.

| | | linker | | linker | | linker | | linker | |
|---|---|---|---|---|---|---|---|---|---|
| Construct ID | BB1 | 1 | BB2 | 2 | BB3 | 3 | BB4 | 4 | BB5 |
| F027300010 | 6C11 | 35GS | ALB | 35GS | 1E07/1 | 35GS | 1E07/1 | | |
| F027300011 | 6C11 | 35GS | ALB | 35GS | 1B11 | 35GS | 1B11 | | |
| F027300012 | 6C11 | 35GS | ALB | 35GS | 15B07 | 35GS | 15B07 | | |
| F027300013 | 1E07/1 | 35GS | ALB | 35GS | 1E07/1 | 35GS | 6C11 | | |
| F027300014 | 1B11 | 35GS | ALB | 35GS | 1B11 | 35GS | 6C11 | | |
| F027300015 | 15B07 | 35GS | ALB | 35GS | 15B07 | 35GS | 6C11 | | |
| F027300016 | 1E07/1 | 35GS | 1E07/1 | 35GS | ALB | 35GS | 6C11 | | |
| F027300017 | 1B11 | 35GS | 1B11 | 35GS | ALB | 35GS | 6C11 | | |
| F027300018 | 15B07 | 35GS | 15B07 | 35GS | ALB | 35GS | 6C11 | | |
| F027300019 | 6C11 | 35GS | 1E07/1 | 35GS | ALB | 35GS | 1E07/1 | | |
| F027300020 | 6C11 | 35GS | 1B11 | 35GS | ALB | 35GS | 1B11 | | |
| F027300021 | 6C11 | 35GS | 15B07 | 35GS | ALB | 35GS | 15B07 | | |
| F027300022 | 1E07/1 | 35GS | 1E07/1 | 35GS | 6C11 | 35GS | ALB | | |
| F027300023 | 1B11 | 35GS | 1B11 | 35GS | 6C11 | 35GS | ALB | | |
| F027300024 | 15B07 | 35GS | 15B07 | 35GS | 6C11 | 35GS | ALB | | |
| F027300025 | 6C11 | 35GS | 1E07/1 | 35GS | 1E07/1 | 35GS | ALB | | |
| F027300026 | 6C11 | 35GS | 1B11 | 35GS | 1B11 | 35GS | ALB | | |
| F027300027 | 6C11 | 35GS | 15B07 | 35GS | 15B07 | 35GS | ALB | | |
| F027300028 | 1E07/1 | 35GS | 1E07/1 | 35GS | 1C02/1 | 9GS | ALB | 9GS | 1C02/1 |
| F027300029 | 1B11 | 35GS | 1B11 | 35GS | 1C02/1 | 9GS | ALB | 9GS | 1C02/1 |
| F027300030 | 15B07 | 35GS | 15B07 | 35GS | 1C02/1 | 9GS | ALB | 9GS | 1C02/1 |
| F027300031 | 1E07/1 | 35GS | 1E07/1 | 35GS | VHH#3E | 9GS | ALB | 9GS | VHH#3E |
| F027300032 | 1B11 | 35GS | 1B11 | 35GS | VHH#3E | 9GS | ALB | 9GS | VHH#3E |
| F027300033 | 15B07 | 35GS | 15B07 | 35GS | VHH#3E | 9GS | ALB | 9GS | VHH#3E |
| F027300034 | 1C02/1 | 9GS | ALB | 9GS | 1C02/1 | 35GS | 1E07/1 | 35GS | 1E07/1 |
| F027300035 | 1C02/1 | 9GS | ALB | 9GS | 1C02/1 | 35GS | 1B11 | 35GS | 1B11 |
| F027300036 | 1C02/1 | 9GS | ALB | 9GS | 1C02/1 | 35GS | 15B07 | 35GS | 15B07 |
| F027300037 | VHH#3E | 9GS | ALB | 9GS | VHH#3E | 35GS | 1E07/1 | 35GS | 1E07/1 |
| F027300038 | VHH#3E | 9GS | ALB | 9GS | VHH#3E | 35GS | 1B11 | 35GS | 1B11 |
| F027300039 | VHH#3E | 9GS | ALB | 9GS | VHH#3E | 35GS | 15B07 | 35GS | 15B07 |
| F027300213 | 1B11 | 35GS | ALB | 35GS | 6C11 | | | | |
| F027300214 | 1B11 | 35GS | 6C11 | 35GS | ALB | | | | |
| F027300215 | 1E07/1 | 35GS | ALB | 35GS | 6C11 | | | | |
| F027300216 | 1E07/1 | 35GS | 6C11 | 35GS | ALB | | | | |
| F027300217 | 15B07 | 35GS | ALB | 35GS | 6C11 | | | | |
| F027300218 | 15B07 | 35GS | 6C11 | 35GS | ALB | | | | |
| F027300219 | 6C11 | 35GS | ALB | 35GS | 1B11 | | | | |
| F027300220 | 6C11 | 35GS | ALB | 35GS | 1E07/1 | | | | |
| F027300221 | 6C11 | 35GS | ALB | 35GS | 15B07 | | | | |
| F027300222 | 6C11 | 35GS | 1B11 | 35GS | ALB | | | | |
| F027300223 | 6C11 | 35GS | 1E07/1 | 35GS | ALB | | | | |
| F027300224 | 6C11 | 35GS | 15B07 | 35GS | ALB | | | | |
| F027300225 | VHH#3E | 9GS | ALB | 9GS | VHH#3E | 35GS | 1B11 | | |
| F027300226 | VHH#3E | 9GS | ALB | 9GS | VHH#3E | 35GS | 15B07 | | |
| F027300227 | 15B07 | 35GS | VHH#3E | 9GS | ALB | 9GS | VHH#3E | | |
| F027300228 | 1E07/1 | 35GS | VHH#3E | 9GS | ALB | 9GS | VHH#3E | | |
| F027300229 | 1E07/1 | 35GS | 1C02/1 | 9GS | ALB | 9GS | 1C02/1 | | |
| F027300230 | VHH#3E | 9GS | ALB | 9GS | VHH#3E | 35GS | 1E07/1 | | |
| F027300231 | 1B11 | 35GS | VHH#3E | 9GS | ALB | 9GS | VHH#3E | | |
| F027300232 | 1C02/1 | 9GS | ALB | 9GS | 1C02/1 | 35GS | 1E07/1 | | |
| F027300233 | 1C02/1 | 9GS | ALB | 9GS | 1C02/1 | 35GS | 1B11 | | |
| F027300234 | 1C02/1 | 9GS | ALB | 9GS | 1C02/1 | 35GS | 15B07 | | |
| F027300235 | 1B11 | 35GS | 1C02/1 | 9GS | ALB | 9GS | 1C02/1 | | |
| F027300236 | 15B07 | 35GS | 1C02/1 | 9GS | ALB | 9GS | 1C02/1 | | |
| F027300237 | 6C11 | 9GS | 15B07 | 9GS | ALB | 9GS | 15B07 | | |
| F027300238 | 1E07/1 | 9GS | 1E07/1 | 9GS | ALB | 9GS | 6C11 | | |
| F027300239 | 1B11 | 9GS | 1B11 | 9GS | ALB | 9GS | 6C11 | | |
| F027300240 | 6C11 | 9GS | 1B11 | 9GS | ALB | 9GS | 1B11 | | |
| F027300241 | 15B07 | 20GS | 15B07 | 9GS | ALB | 9GS | 6C11 | | |
| F027300242 | 15B07 | 9GS | ALB | 9GS | 15B07 | 9GS | 6C11 | | |
| F027300243 | 6C11 | 9GS | ALB | 9GS | 15B07 | 20GS | 15B07 | | |
| F027300244 | 6C11 | 9GS | ALB | 9GS | 1E07/1 | 9GS | 1E07/1 | | |
| F027300245 | 6C11 | 9GS | ALB | 9GS | 1B11 | 9GS | 1B11 | | |
| F027300246 | 1E07/1 | 9GS | 1E07/1 | 9GS | 6C11 | 9GS | ALB | | |
| F027300247 | 1B11 | 9GS | 1B11 | 9GS | 6C11 | 9GS | ALB | | |
| F027300248 | 15B07 | 20GS | 15B07 | 9GS | 6C11 | 9GS | ALB | | |
| F027300249 | 6C11 | 9GS | 1E07/1 | 9GS | 1E07/1 | 9GS | ALB | | |
| F027300250 | 6C11 | 9GS | 1B11 | 9GS | 1B11 | 9GS | ALB | | |
| F027300251 | 6C11 | 9GS | 15B07 | 20GS | 15B07 | 9GS | ALB | | |
| F027300252 | 1E07/1 | 9GS | 1E07/1 | 9GS | 1C02/1 | 9GS | ALB | 9GS | 1C02/1 |
| F027300253 | VHH#3E | 9GS | ALB | 9GS | VHH#3E | 9GS | 1B11 | 9GS | 1B11 |
| F027300254 | 1C02/1 | 9GS | ALB | 9GS | 1C02/1 | 9GS | 15B07 | 20GS | 15B07 |
| F027300255 | VHH#3E | 9GS | ALB | 9GS | VHH#3E | 9GS | 15B07 | 20GS | 15B07 |
| F027300256 | 1B11 | 9GS | 1B11 | 9GS | 1C02/1 | 9GS | ALB | 9GS | 1C02/1 |
| F027300257 | 1E07/1 | 9GS | 1E07/1 | 9GS | VHH#3E | 9GS | ALB | 9GS | VHH#3E |
| F027300258 | 1B11 | 9GS | 1B11 | 9GS | VHH#3E | 9GS | ALB | 9GS | VHH#3E |
| F027300259 | 15B07 | 20GS | 15B07 | 9GS | 1C02/1 | 9GS | ALB | 9GS | 1C02/1 |
| F027300260 | VHH#3E | 9GS | ALB | 9GS | VHH#3E | 9GS | 1E07/1 | 9GS | 1E07/1 |
| F027300610 | 1B11 | 9GS | ALB | 9GS | 1B11 | 9GS | 6C11 | | |
| F027300659 | 1C02/1 | 9GS | ALB | 9GS | 1C02/1 | 9GS | 1E07/1 | 9GS | 1E07/1 |
| F027300660 | 1C02/1 | 9GS | ALB | 9GS | 1C02/1 | 9GS | 1B11 | 9GS | 1B11 |
| F027300841 | 15B07 | 20GS | 15B07 | 9GS | VHH#3E | 9GS | ALB | 9GS | VHH#3E |
| F027301194 | 1E07/1 | 9GS | 6C11 | 9GS | ALB | | | | |
| F027301195 | 1E07/1 | 9GS | ALB | 9GS | 6C11 | | | | |

Some constructs showed impaired potencies depending on valency, linker length, and relative position of ISVD building blocks. For example: considerable differences in OX40L potencies were observed for 6 bispecific ISVD constructs although they were comprising the same building blocks targeting OX40L and TNFα. The exact composition (valency, orientation of building blocks and usage of linker lengths) was found to be critical for potency. The listed potencies for OX40L blocking as shown in table 3 demonstrate the importance of bivalency and N-terminal position of the anti-OX40L 1E07/1 building block.

TABLE 3
IC50 values of neutralization of human and cyno OX40L in the PBMC activity assay for
multispecific ISVDs with same building blocks as ISVD construct F027300052 versus the reference
compound anti-hOX40L mAb.

| | | | | | | | | | | | Ratio IC50 |
|---|---|---|---|---|---|---|---|---|---|---|---|
| | | | | | | | | | | | anti- |
| | | | | | | | | | | OX40L | hOX40L |
| | | | | | | | | | | PBMC | reference |
| | | | | | | | | | | assay | mAb/ |
| Construct | | linker | | linker | | linker | | linker | | (IC50, | ISVD |
| ID | BB1 | 1 | BB2 | 2 | BB3 | 3 | BB4 | 4 | BB5 | pM) | construct |
| F027300229 | 1E07/1 | 35GS | 1C02/1 | 9GS | ALB | 9GS | 1C02/1 | | | 51500 | 0.09 |
| F027300232 | 1C02/1 | 9GS | ALB | 9GS | 1C02/1 | 35GS | 1E07/1 | | | 86000 | 0.05 |
| F027300252 | 1E07/1 | 9GS | 1E07/1 | 9GS | 1C02/1 | 9GS | ALB | 9GS | 1C02/1 | 1090 | 10.0 |
| F027300659 | 1C02/1 | 9GS | ALB | 9GS | 1C02/1 | 9GS | 1E07/1 | 9GS | 1E07/1 | 4820 | 0.50 |
| F027301097 | 1E07/1 | 35GS | 1E07/1 | 35GS | 1C02/1 | 9GS | ALB | 9GS | 1C02/1 | 866 | 2.00 |
| F027301100 | 1C02/1 | 9GS | ALB | 9GS | 1C02/1 | 35GS | 1E07/1 | 35GS | 1E07/1 | 6520 | 0.67 |

Subsequently, the large panel was trimmed down to a panel of five multispecific constructs, consisting of ISVD constructs F027300252, F027301140, F027301189, F027301197 and F027301199, proven to be potent on both targets (human and cyno) and comprising the potential of high expression levels, based on preliminary yield estimates.

Larger scale 2 L and 5 L productions in Pichia pastoris of the panel comprising the 5 ISVD constructs were done for expression yield determination, assessment of biophysical properties, and pre-existing reactivity. It was demonstrated that a specific combination of the anti-OX40L building blocks and anti-TNFα building blocks is required to obtain high expression yields in Pichia pastoris as well as sufficient solubility and biophysical stability. As exemplified in table 5, comparing ISVD constructs F027300252 and F07301199, already the use of anti-TNF building block resulted in a largely different CMC profile. Upon 5 L fermentation the ISVD construct F027300252 not only reached a titer of 6 g/l which is 3-fold higher than for ISVD construct F07301199, but also exhibited superior storage properties and viscosity.

TABLE 4
Building block composition of ISVD constructs F027300252 and F07301199.

| Construct ID | BB1 | linker 1 | BB2 | linker 2 | BB3 | linker 3 | BB4 | linker 4 | BB5 |
|---|---|---|---|---|---|---|---|---|---|
| F027300252 | 1E07/1 | 9GS | 1E01 | 9GS | 1C02/1 | 9GS | ALB | 9GS | 1C02/1 |
| F027301104 | 6C11 | 35GS | 1B11 | 35GS | 1B11 | 35GS | ALB | | |
| F027301189 | 15B07 | 20GS | 15B07 | 9GS | ALB | 9GS | 6C11 | | |
| F027301197 | 6C11 | 9GS | 1B11 | 9GS | 1B11 | 9GS | ALB | | |
| F027301199 | 1E07/1 | 9GS | 1E07/1 | 9GS | VHH#3E | 9GS | ALB | 9GS | VHH#3E |

TABLE 5
Expression yields and biophysical properties of pentavalent ISVD constructs F0273000252 and
F027301199.

| | Expression | Expression | | | | | |
|---|---|---|---|---|---|---|---|
| | yield 5 ml | yield 5 L | | | | Freeze- | |
| | culture | fermentor | solubility | Storage at | Storage at | thaw | Viscosity |
| Construct ID | (μg/ml) | (g/L) | (mg/ml) | −60° C.* | 5° C.* | stability* | (cP)* |
| F027300252 | 387 | 6.0 | 152 | No change | No change | No change | 8.0 |
| F027301104 | 140 | 4.3 | 154 | No change | SVP after 2-3 | No change | 16.8 |
| | | | | | weeks | | |
| F027301189 | 190 | 3.4 | 151 | No change | No change | No change | 14.1 |
| F027301197 | 80 | 4.6 | 144 | No change | SVP after 2-3 | No change | 9.6 |
| | | | | | weeks | | |
| F027301199 | 50 | 2.0 | 158 | turbidity | Phase | turbidity | 19.3 |
| | | | | | separation, SVP | | |

*at high concentration of >150 mg/ml. ALB = ALB23002, BB = building block, SVP subvisible particle

Further, table 6 and example 12 demonstrate that the pre-existing antibody reactivity is driven by the composition, valency, and linker lengths of the respective ISVD constructs.

TABLE 6
Binding of pre-existing antibodies present in 96 human serum samples to F027300252,
F027301140, F027301189, F027301197 and F027301199 compared to control ISVD
constructs F027301099 and F027301186.

| | | linker | | linker | | linker | | linker | | 25% | Median | 75% |
|---|---|---|---|---|---|---|---|---|---|---|---|---|
| Construct ID | BB1 | 1 | BB2 | 2 | BB3 | 3 | BB4 | 4 | BB5 | percentile | RU level | percentile |
| F027300252 | 1E07/1 | 9GS | 1E07/1 | 9GS | 1C02/1 | 9GS | ALB | 9GS | 1C02/1-A | −8 | 10 | 29 |
| F027301104 | 6C11 | 35GS | 1B11 | 35GS | 1B11 | 35GS | ALB- | | | 30 | 56 | 85 |
| | | | | | | | A | | | | | |
| F027301189 | 15B07 | 20GS | 15B07 | 9GS | ALB | 9GS | 6C11- | | | −13 | −4 | 9 |
| | | | | | | | A | | | | | |
| F027301197 | 6C11 | 9GS | 1B11 | 9GS | 1B11 | 9GS | ALB- | | | 22 | 32 | 47 |
| | | | | | | | A | | | | | |
| F027301199 | 1E07/1 | 9GS | 1E07/1 | 9GS | VHH# | 9GS | ALB | 9GS | VHH# | nt | nt | nt |
| | | | | | 3E | | | | 3E-A | | | |
| F027301099 | 1B11 | 35GS | ALB | 35GS | 1B11 | 35GS | 6C11 | | | 47 | 92 | 350 |
| F027301186 | 1E07/1 | 35GS | 1E07/1 | 35GS | 1C02/1 | 9GS | ALB | 9GS | 1C02/1 | 61 | 135 | 622 |

BB = building block, nt = not tested

Finally, ISVD construct F027300252 was selected based on potency, reduced binding to preexisting antibodies, superior expression levels and CMC characteristics and reduced binding to pre-existing antibodies.

### 6.2 Example 2: Multispecific ISVD Construct Binding Affinity to TNFα, OX40L, and Serum Albumin

The affinity, expressed as the equilibrium dissociation constant (K_{D}), of F027300252 towards human, cynomolgus monkey, guinea pig and mouse TNFα, human and cyno OX40L, and human and cyno serum albumin was quantified by means of in-solution affinity measurements on a Gyrolab xP Workstation (Gyros).

Under K_{D}-controlled measurements a serial dilution of TNFα or OX40L (ranging from 1 μM-0.1 pM) or serum albumin (ranging from 10 μM-1 pM) and a fixed amount of F027300252 (20 pM in case of TNFα, 30 pM in case of OX40L and 300 pM in case of serum albumin) were mixed to allow interaction and incubated for either 24 or 48 hours (in case of OX40L and TNFα) or 2 hours (in case of serum albumin) to reach equilibrium.

Under receptor-controlled measurements a serial dilution of TNFα or OX40L (ranging from 1 μM-0.1 pM) and a fixed amount of F027300252 (5 nM in case of TNFα and 5 nM in case of OX40L were mixed to allow interaction and incubated for either 24 or 48 hours to reach equilibrium.

Biotinylated human TNFα/OX40L/serum albumin was captured in the microstructures of a Gyrolab Bioaffy 1000 CD, which contains columns of beads and is used as a molecular probe to capture free F027300252 from the equilibrated solution. The mixture of TNFα/OX40L/serum albumin and F027300252 (containing free TNFα/OX40L/serum albumin, free F027300252 and TNFα/OX40L/serum albumin—F027300252 complexes) was allowed to flow through the beads, and a small percentage of free F027300252 was captured, which is proportional to the free ISVD construct concentration. A fluorescently labeled anti-V_{HH} antibody, ABH0086-Alexa647, was then injected to label any captured F027300252 and after rinsing away excess of fluorescent probe, the change in fluorescence was determined. Fitting of the dilution series was done using Gyrolab Analysis software, where K_{D}- and receptor-controlled curves were analyzed to determine the K_{D} value.

The results (Table 7) demonstrate that the multispecific ISVD construct binds human/cyno

OX40L and human/cyno TNFα with high affinity.

TABLE 7
Binding affinities of F027500252 to human and cyno OX40L,
TNFa and serum albumin.

| | human | | cynomolgus monkey | | |
|---|---|---|---|---|---|
| | K_{D} | 95% | K_{D} | 95% CI | Incubation |
| Antigen | (pM) | CI (pM) | (pM) | (pM) | time (h) |
| OX40L | 49.7 | 32.2-67.1 | 8.3 | 3.1-13.5 | 24 |
| | 28.2 | 11.7-44.6 | 9.8 | 7.5-12.0 | 48 |
| TNFα | 8.9 | 6.4-11.5 | 41.3 | 23.0-59.6 | 24 |
| | 8.6 | 4.6-12.5 | 34.3 | 28.4-40.2 | 48 |
| SA | 10700 | 8800-12600 | 11200 | 9700-12700 | 2 |

### 6.3 Example 3: Multispecific ISVD Construct Binding to Membrane Bound TNFα

Binding of F027300252 to membrane bound TNFα was demonstrated using flow cytometry on human membrane TNFα expressing HEK293H cells and on activated CD4+ cells that were isolated from PBMC's and stimulated with PMA and lonomycin (data shown for TNFα expressing HEK293H cells). Briefly, cells were seeded at a density of 1×10⁴ cells/well and incubated with a dilution series of F027300252 or reference compound anti-hTNFα mAb, starting from 100 nM up to 0.5 pM, for 1 hour at 4° C. In parallel, cells were fixed with 4% paraformaldehyde and 0.1% glutaraldehyde in PBS, before seeding (to increase detection of membrane bound TNFα), and incubated with a dilution series of ISVD construct or reference compound for 1 hour at 4° C. or for 24 hours at room temperature. Cells were washed 3 times and subsequently incubated with an anti-v_{HH} mAb (ABH00119) for 30 min at 4° C., washed again, and incubated for 30 min at 4° C. with a goat anti-mouse or anti-human PE labeled antibody. Samples were washed and resuspended in FACS Buffer (D-PBS with 10% FBS and 0.05% sodium azide supplemented with 5 nM TOPRO3). Cell suspensions were then analyzed on an iQuescreener. EC50 values were calculated using GraphPad Prism. EC50 values for F027300252 and anti-hTNFα reference mAb are in the same range for viable and fixed cells after 1 hour incubation, though fixation of the cells results in the presence of higher levels of TNFα on the membrane (Table 8). After 24 hours incubation, binding equilibrium was reached. Affinities of F027300252 and anti-hTNFα reference mAb are comparable.

TABLE 8
Binding affinity of F027300252 to membrane expressed
TNFα after incubation times of 1 hour or
24 hours, compared to the reference compound anti-hTNFα mAb.

| | T = 1 h viable cells | T = 1 h fixed cells | T = 24 h fixed cells |
|---|---|---|---|
| Analyte | EC50 (M) | EC50 (M) | EC50 (M) |
| F027300252 | 9.04 E−11 | 1.47 E−10 | 2.02 E−11 |
| anti-hTNFα | 7.08 E−11 | 2.61 E10 | 1.28 E−11 |
| reference | | | |
| mAb | | | |

### 6.4 Example 4: Multispecific ISVD Construct Binding to Membrane Bound OX40L

Binding of F027300252 to membrane bound human and cyno OX40L was demonstrated using flow cytometry on CHO-KI cells expressing human or cyno OX40L. Briefly, cells were fixed with 4% paraformaldehyde and 0.1% glutaraldehyde in PBS, seeded at a density of 1×10⁴ cells/well and incubated with a dilution series of ISVD construct F027300252 or the reference compound anti-hOX40L mAb starting from 100 nM up to 0.5 pM, for 48 hours at room temperature. Cells were washed 3 times and subsequently incubated with an anti-V_{HH} mAb for 30 min at 4° C., washed again, and incubated for 30 min at 4° C. with a goat anti-mouse PE or FITC labeled antibody. Samples were washed and resuspended in FACS Buffer (D-PBS with 10% FBS and 0.05% sodium azide supplemented with 5 nM TOPRO3). Cell suspensions were then analyzed on an iQuescreener. EC50 values were calculated using GraphPad Prism. F0273000252 shows better binding to membrane bound OX40L than the reference compound anti-hOX40L reference mAb with a factor of more than 10 (Table 9).

TABLE 9
Binding affinity of F027300252 to membrane
expressed human and cyno OX40L compared to
reference compound anti-hOX40L reference mAb.

| | human OX40L | cyno OX40L |
|---|---|---|
| Analyte | EC50 (M) | EC50 (M) |
| F027300252 | 1.33E−11 | 1.18E−11 |
| anti-hOX40L | 1.80E−10 | 1.42E−10 |
| reference | | |
| mAb | | |

### 6.5 Example 5: Multispecific ISVD Construct Binds Selectively to TNFα and OX40L

Absence of binding to TNFα and OX40L related human targets was assessed via SPR (Proteon XPR36). As OX40L related targets, human TRAIL, CD30L, CD40L and RANKL were assessed. TNF superfamily members human FASL, TNFβ, LIGHT, TL-1A, RANKL were tested as related cytokines for TNFα.

To this end, the TNF related cytokines were immobilized on a Proteon GLC sensor chip at 25 μg/mL for 200 s using amine coupling, with 80 seconds injection of EDC/NHS for activation and a 150 seconds injection of 1 M ethanolamine HCI for deactivation (ProteOn Amine Coupling Kit. cat. 176-2410). Flow rate during activation, deactivation and ligand injection was set to 30 μl/min. The pH of the 10 mM acetate immobilization buffer was chosen by subtracting ˜1.5 from the pl of each ligand.

Next, 10 nM or 300 nM of F027300252 was injected for 2 minutes and allowed to dissociate for 900 s at a flow rate of 45 μL/min. As running buffer PBS (pH7.4)+0.005% Tween 20 was used. As positive controls, 0.3 μM α-hFASL Ab, 0.3 μM α-hTNFβ Ab, 0.5 μM α-hLIGHT Ab and 0.3 μM α-hTL-1A Ab were injected. Interaction between F027300252 and the positive controls with the immobilized targets was measured by detection of increases in refractory index which occurs as a result of mass changes on the chip upon binding.

In the case of the OX40L related targets, ISVD construct F027500252 or the positive control antibodies α-hTRAIL, α-hCD30L, α-hCD40L and α-hRANKL V_{HH} were immobilized on the sensor chip at 10 μg/ml.

Next 1 μM of human TRAIL, CD30L, CD40L and RANKL was injected for 2 minutes and allowed to dissociate for 900 s at a flow rate of 45 μL/min.

All positive controls did bind to their respective target. No binding was detected of ISVD construct F027300252 to human TRAIL, CD30L, CD40L, FASL, TNFβ, LIGHT, TL-1A and RANKL.

### 6.6 Example 6: Simultaneous Binding of Multispecific ISVD Construct to hOX40L, hTNFα, and HSA

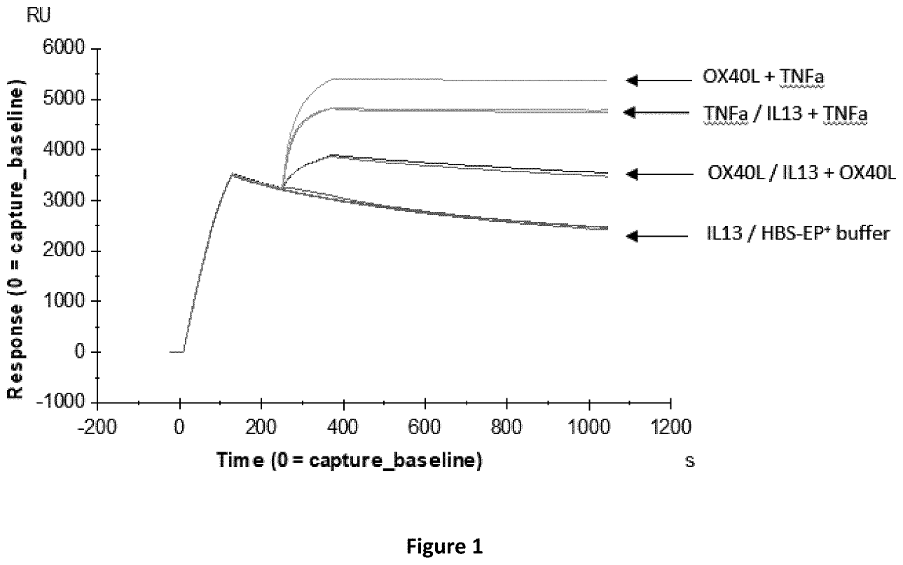

A Biacore T200 instalment was used to determine whether ISVD construct F0273000252 can bind simultaneously to recombinant soluble hTNFα and hOX40L. To this end HSA was immobilized on a CM5 sensor chip via amine coupling to a level of 6000 RU. 100 nM of F0273000252 was injected for 2 min at 10 μl/min over the HSA surface in order to capture the ISVD construct via the ALB23002 building block. Subsequently either 100 nM of hOX40L, hTNFα or hIL13 were injected or mixtures of 100 nM OX40L+100 nM TNFα, 100 nM IL13+100 nM OX40L or 100 nM TNFα+100 nM IL13 at a flow rate of 45 μl/min for 2 min followed by a subsequent 600 seconds dissociation step. The HSA surfaces are regenerated with a 2-minute injection of HCl (100 mM) at 45 μl/min. The sensorgram (FIG. 1) demonstrates that ISVD construct F027300252 can bind hOX40L and hTNFα simultaneously as shown by the increase in response units after capture on HSA: ˜1770 RU increase from hTNFα only, ˜800 RU increase from hOX40L only and ˜2300 RU increase for the OX40L and TNFα mixture.

Using flow cytometry, it was determined whether ISVD construct F0273000252 can bind simultaneously to recombinant soluble hTNFα and cell membrane bound hOX40L. To this end, CHO-KI cells expressing human OX40L were seeded at a density of 5×10⁴ cells/well and incubated with 100 nM ISVD construct F027300252 for 90 minutes at 4° C. Subsequently the mixture was incubated with a dilution series of biotinylated TNFα starting from 500 nM up to 7.6 pM, and incubated for 30 min at 4° C., in the presence of 30 μM HSA. Cells were washed 3 times and subsequently incubated with PE-labelled anti-streptavidin for 30 min at 4° C., washed again. Samples were washed and resuspended in FACS Buffer (D-PBS with 10% FBS and 0.05% sodium azide supplemented with 5 nM TOPRO3). Cell suspensions were then analyzed on an iQuescreener. The dose-response curve (FIG. 2) demonstrated that ISVD construct F027300252 can bind membrane bound hOX40L and soluble hTNFα simultaneously in the presence of HAS, whereas the negative control V_{HH}, IRR0096, cannot bind.

### 6.7 Example 7: Multispecific ISVD Construct Inhibition of TNFα-Induced NFkB Activation In Vitro

HEK293_NFκB-NLucP cells are TNF receptor expressing cells that were stably transfected with a reporter construct encoding Nano luciferase under control of a NFκB dependent promoter. Incubation of the cells with soluble human and cyno TNFα resulted in NFκB mediated Nano luciferase gene expression. Nano luciferase luminescence was measured using Nano-Glo Luciferase substrate mixed with lysing buffer at the ratio of 1:50 added onto cells. Samples were mixed 5 min on a shaker to obtain complete lysis.

Glo response™ HEK293_NFκB-NLucP cells were seeded at 20000 cells/well in normal growth medium in white tissue culture (TC) treated 96-well plates with transparent bottom. Dilution series of F0273000252 or reference compound (anti-hTNFα mAb) were added to 25 pM human or 70 pM cyno TNFα and incubated with the cells for 5 hours at 37° C. in the presence of 30 μM HSA.

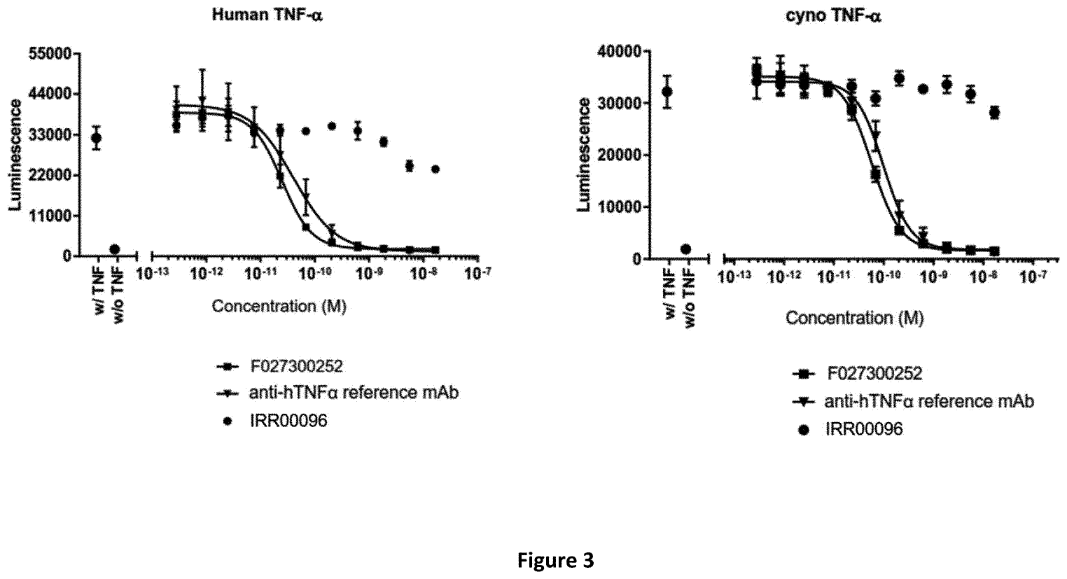

F027300252 inhibited human and cyno TNFα-induced NFκB activation in a concentration-dependent manner with an IC50 of 31 pM (for human TNFα) and 91 pM (for cyno TNFα) comparable to the reference compound anti-hTNFα mAb (Table 10, FIG. 3). The negative control V_{HH}, IRR00096, did not show inhibition.

TABLE 10
IC50 values of F0275000252 mediated neutralization
of human and cyno TNFα in the Glo
response ™ HEK293_NFκB-NLucP reporter assay
versus the reference compound anti-hTNFα mAb.

| | F02750069 | | anti-hTNFα reference mAb | |
|---|---|---|---|---|
| antigen | human TNFα | cyno TNFα | human TNFα | cyno TNFα |
| NFkB assay | 3.1E−11 | 9.1-11 | 5.5E−11 | 8.6E−11 |
| IC50 (M) | | | | |

### 6.8 Example 8: Inhibition of TNFα by Multispecific ISVD Construct Reduces Luciferase Expression in a Stable NFkB Luciferase Reporter Cell Line

For measuring the neutralization of TNFα by mono- or multispecific antibodies or ISVD constructs/V_{HH}s the NFκB reporter stable cell line A549/NFκB-luc (cat. #RC002) was used. Nuclear Factor kappa B (NFκB) is a member of the rel family of transcription factors and plays a key role in the regulation of inflammatory response, apoptosis or tumorigenesis. The cell line used here is derived from human lung carcinoma cells A549 with chromosomal integration of a luciferase reporter construct regulated by 6 copies of the NFκB response element. With this cell line any changes occurring along the NFκB pathway can be accurately monitored.

The ISVD construct potency was determined in dose response of 10 serially diluted concentrations by neutralizing human TNFα (SIGMA #H8916) and cyno TNFα (Sino 90018-CNAE-5) at their EC90. Human TNFα was used at [15 ng/ml], and cyno TNFα was used at [10 ng/ml].

The thaw-and-use A549/NFκB-luc cells were resuspended in RPMI medium containing 1% FCS and seeded in 384-well plate each well with 10K cells in 10 μl. 10 μl of the anti-TNF/anti-OX40L multispecific ISVD construct F027300252, or the corresponding positive and negative control antibodies and V_{HH} were diluted in the RPMI medium and added to the cells. An anti-TNFα antibody produced in-house (anti-TNFα mAb2) was used as positive control, and the V_{HH} IRR00119 as well as the antibody RA11093885 were used as negative controls. After preincubation for 15 minutes at room temperature, 10 μl of TNFα in a concentration of 15 ng/ml was added to the wells. The whole reaction was terminated after 5 hours at 37° C. with 5% CO2 and 95% humidity by the addition of 20 μl Bio-Glo luciferase detection reagents (Promega E7940). The luminescence signal was measured by PheraStar (BMG). The XLfit program in Speed was used for fitting the dose response curves and calculating the IC50 values.

TABLE 11
Mean IC50 results.

| | mean IC50 [nM] | mean IC50 [nM] |
|---|---|---|
| | (@15 ng/ml human | (@10 ng/ml |
| Compound ID | TNF) | cyno TNF) |
| F027300252 | 0.22 | 0.21 |
| anti-TNFα mAb2 | 0.2 | 0.13 |
| IRR00119 (negative control V_{HH}) | no inhibition | no inhibition |
| RA11093885 (negative control IgG) | no inhibition | no inhibition |

### 6.9 Example 9: Multispecific ISVD Construct Inhibition of OX40L Induced Co-Stimulation of T Cells In Vitro

Functional activity of human and cyno OX40L and inhibition thereof by ISVD construct F027300252 was studied using a cell-based assay, investigating OX40L induced co-stimulation of T-cells (PBMC activity assay). The assay was performed by co-culturing buffy coat derived PBMC (at a density of 1×10⁵ cells/well) in the presence of suboptimal concentration of PHA-L (to induce OX40 expression) with CHO-KI cells overexpressing OX40L (at a density of 1×10⁴cells/well) in transparent 96-well plates. A dilution series of ISVD construct F027300252 or reference compound anti-hOX40L mAb was added to the co-culture and incubated in the presence of 30 μM HSA for 22 hours at 37° C. in a humidified incubator. Readout was performed by evaluating IL2 levels in the supernatant of these cells using ELISA.

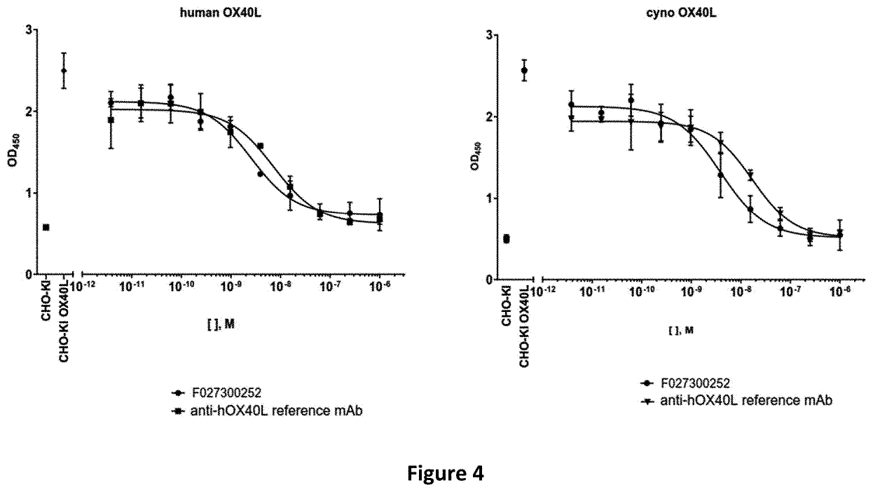

ISVD construct F027300252 inhibited human and cyno OX40L-induced T-cell activation in a concentration-dependent manner with an IC50 of 2.58 nM (for human OX40L) and 7.22 nM (for cyno OX40L) comparable to the reference compound anti-hOX40L mAb (Table 12, FIG. 4).

TABLE 12
IC50 values of F0273000252 mediated neutralization
of human and cyno OX40L in the PBMC
activity assay versus the reference compound anti-hOX40L mAb.

| | F02750069 | | a nti-hOX40L reference mAb | |
|---|---|---|---|---|
| | | Cyno | Human | Cyno |
| antigen | Human OX40L | OX40L | OX40L | OX40L |
| PBMC activity | 2.58E−09 | 3.89E−09 | 7.22E−09 | 1.72E−08 |
| assay | | | | |
| IC50 (M) | | | | |

### 6.10 Example 10: Inhibition of TNFα and OX40L by the Multispecific ISVD Construct F027300252 Reduces Luciferase Expression in a Stable NFkB Luciferase Reporter Cell Line

For measuring the neutralization of TNFα and OX40L individually or in combination by mono- or multispecific antibodies or ISVD constructs/V_{HH}s, the NFκB reporter stable cell line Jurkat NF-κB Luc2/OX40 was used. Nuclear Factor kappa B (NFκB) is a member of the rel family of transcription factors and plays a key role in the regulation of inflammatory response, apoptosis or tumorigenesis. The cell line used here was derived from a human peripheral blood T lymphocyte, with chromosomal integration and stably expressing human OX40 receptor and a codon optimized firefly luciferase reporter gene luc2 construct regulated by 6 copies of the NFκB response element. With this cell line, any changes occurring along the NFκB pathway can be accurately monitored. The thaw-and-use Jurkat NF-κB Luc2/OX40 cells were resuspended in RPMI medium containing 1% FCS, and seeded in 96-well plate each well with 1×105 cells/ml for culture.

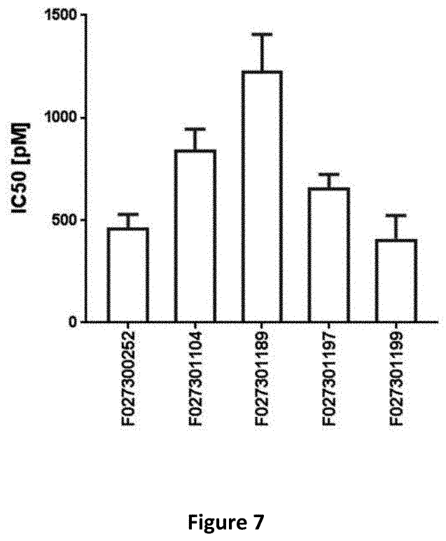

At the begin of the assay, the recombinant human TNFα and human OX40L were added to a final concentration of 5 ng/ml and 100 ng/ml to the wells of 96 well Eppendorf suspension culture plates in 85 μl/well, and 85 μl of the pre-diluted anti-TNFα antibody PB03017 (from Sanofi), the anti-OX40L antibody (Cat #AB00536 from Absolute Antibody), the IgG1 isotype negative control antibody (Cat #403502 from Biolegend), the negative control V_{HH} IRR00119 (from Sanofi), the monospecific anti-TNFα V_{HH} ATN-103 (from Sanofi), the monospecific anti-OX40L V_{HH} ALX-0632 (from Sanofi), or the anti-TNF/anti-OX40L multispecific ISVD constructs F027300252, F027301104, F027301189, F027301197, and F027301199 (all from Sanofi) where added. After preincubation for 15 minutes at 37° C., 75 μl of that mixture where added/well to 50 μl of 1×10⁵ cells/well and incubated for 6 hours at 37° C. with 5% CO2 and 95% humidity. The reaction was stopped by the addition of 125 μl Bio-Glo luciferase detection reagents (Promega E7940) and the luminescence signal was measured. The XLfit program in Speed was used for fitting the dose response curves and calculating the IC50 values in FIG. 7.

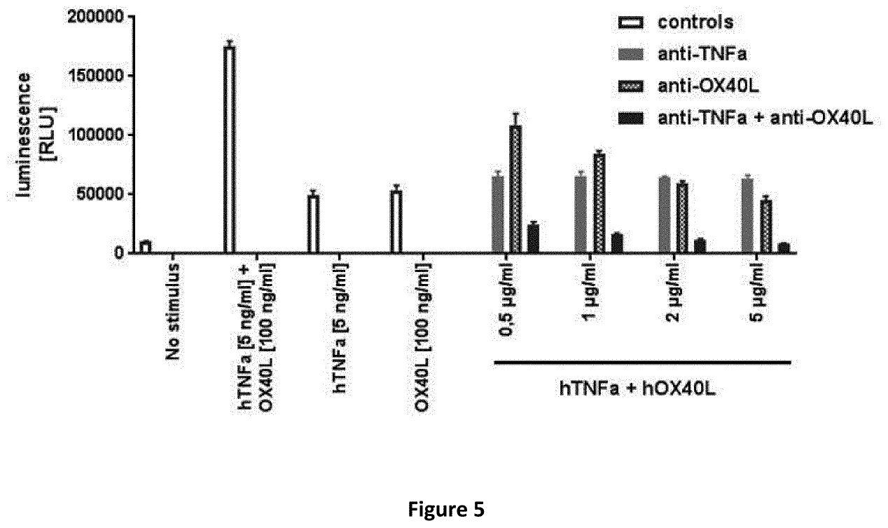

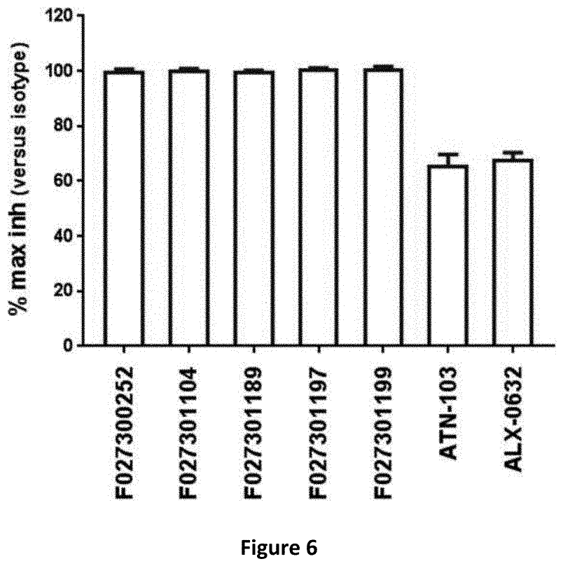

Additive effects of the anti-TNFα and anti-OX40L combination in comparison to potency of the individual arms was determined at antibody doses of 0.5 μg/ml, 1 μg/ml, 2 μg/ml, and 5 μg/ml (FIG. 5). The IC50 (FIG. 7, table 13) and %-inhibition (FIG. 6, table 13) of the multispecific ISVD constructs in comparison to the monospecific V_{HH}s was determined in dose response of 9 serially diluted concentrations by neutralizing recombinant human TNFα (Cat #H8916 from Sigma) and recombinant human OX40L (Cat #71185 from bpsbioscience) at their EC90 their EC90 to achieve comparable levels of luciferase induction by each individual stimulus (see FIG. 5). Human TNFα was used at 5 ng/ml, and human OX40L was used at 100 ng/ml final assay concentration, respectively.

Incubation with both, recombinant human TNFα (hTNFα) and recombinant human OX40L (OX40L) leads to a much stronger induction (>3 fold) of luciferase activity compared to treatment with either stimulus alone (see FIG. 5). Incubation with both, recombinant human TNFα and recombinant human OX40L (hTNFα+OX40L), was used in order to characterize the effect of inhibition by an anti-TNFα antibody alone, inhibition by an anti-OX40L antibody alone, or a combination of an anti-TNFα antibody and anti-OX40L antibody at different concentrations in the range of 0.5 μg/ml up to 5 μg/ml (FIG. 5). While treatment with anti-TNFα or anti-OX40L alone did lead to some inhibition of the luciferase activity induced by combined hTNFα+OX40L, only treatment with combined anti-TNFα/anti-OX40L was able to very strongly suppress luciferase activity to a very low level corresponding to the control without stimulus (FIG. 5). Thus, combined treatment with anti-TNF/anti-OX40L even at very low concentrations of e.g. 0.5 μg/ml or 1 μg/ml far more potently suppressed luciferase activity compared to treatment with anti-TNFα or anti-OX40L alone at a high concentration of 5 μg/ml (FIG. 5).

Similarly, incubation with the combination of recombinant human TNFα and recombinant human OX40L (hTNFα+OX40L) was used in order to characterize the effect of inhibition by the monospecific anti-OX40L V_{HH} ALX-0632, or the monospecific anti-TNFα V_{HH} ATN-103, or the anti-TNFα/anti-OX40L bispecific ISVD constructs F027300252, F027301104, F027301189, F027301197, and F027301199 at different concentrations in the range of 1 pM up to 20 nM (FIG. 6 and FIG. 7). While treatment with an anti-TNFα V_{HH} or an anti-OX40L V_{HH} alone did lead to some inhibition of the luciferase activity induced by combined hTNFα+OX40L up to a level of approx <70% maximum, only treatment with the bispecific anti-TNFα/anti-OX40L ISVD constructs was able to completely suppress induction of luciferase activity up to a level of 100% (FIG. 6). Furthermore, calculation of IC50 values for the anti-TNFα/anti-OX40L bispecific ISVD constructs F027300252, F027301104, F027301189, F027301197, and F027301199 at different concentrations in the range of 1 pM up to 20 nM showed that the ISVD constructs F027300252 and F027301199 had the strongest potency in this specific set of experiments (FIG. 7).

TABLE 13
IC50 values and % inhibition of ISVD constructs
F027300252, F027301104, F027301189,
F027301197, and F027301199 in the Jurkat OX40/TNF-NFκB
reporter assay (see also FIGS. 6 and 7).

| Construct ID | IC50 (pM) | % inhibition |
|---|---|---|
| F027300252 | 438 | 100 |
| F027301104 | 844 | 100 |
| F027301189 | 1230 | 100 |
| F027301197 | 659 | 100 |
| F027301199 | 374 | 100 |
| ATN-103 | 387 | 66 |
| ALX-0632 | 3065 | 68 |

### 6.11 Example 11: Inhibition of OX40L and TNFα by Multispecific ISVD Construct Reduces GM-CSF Levels in Mixed Lymphocyte Reaction (MLR)

In order to test the physiological effects of OX40L blockade on T cell activation, mixed lymphocyte reaction assays were conducted. Briefly, monocyte-derived dendritic cells (MoDCs) from a healthy blood donor were matured in vitro to express OX40L and these cells were then mixed with PBMCs from another, unrelated, healthy donor. Mixing unrelated donors in the same well induced allo-reaction and T cell activation. This allogenic T cell response was monitored by means of cytokine measurement in the supernatant 5 days after mixing the cells. Below, a detailed explanation for the preparation of MoDCs and evaluation of T cell response by cytokine measurement is shown.

Preparation of Monocyte-Derived Dendritic Cells from PBMCs of Healthy Blood Donors:

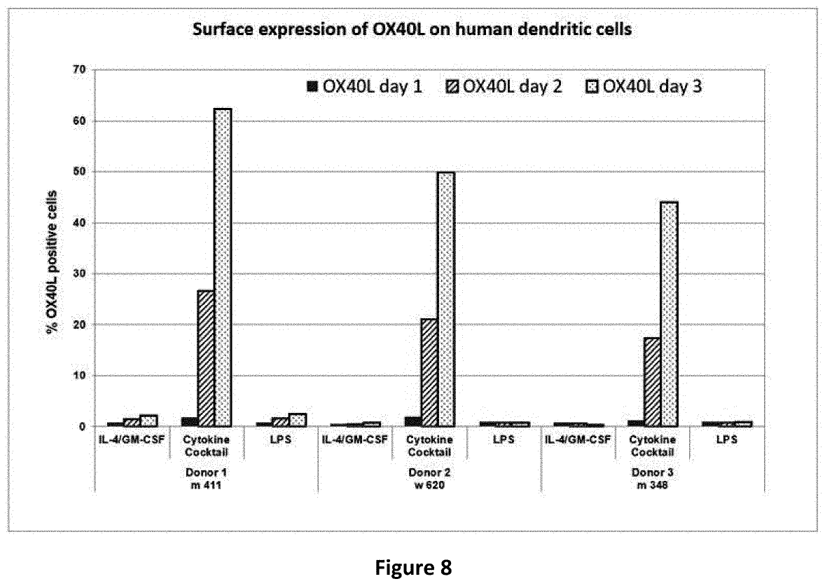

PBMCs were isolated from whole blood or buffy coats via gradient centrifugation. Cells were counted and 3×10⁷ cells were plated per well of 6-well plate in 3 ml RPMI 1640 medium containing Glutamax, 10% human serum, 10 mM Hepes and 20 μg/ml Gentamicin. 1-2 hours later, non-adherent cells were washed with 3 rounds of washing and the cells were incubated for 5 days in the presence of 500 IU/ml of IL-4 and 500 IU/ml of GM-CSF. At the 3^{rd} day of this 5 day incubation, the medium was partially replaced with fresh medium containing IL-4 and GM-CSF. At the end of the 5-day incubation period, differentiated but still immature DCs were collected and counted. The DCs were then re-plated for further maturation (5×10⁵ cells/ml) in 6-well or 24-well plates. The cells were incubated in medium (same as above) containing a novel cytokine cocktail [500 IU/ml of IL-4, 500 IU/ml of GM-CSF, 10 ng/mL IL-1b, 1000 IU IL-6, 10 ng/mL TNFα, 1 μg/mL PGE2] that has been identified as most suitable one amongst other stimuli to induce expression of OX40L on DCs (FIG. 8). At days 2, 3 and 4 of maturation, DCs were collected and evaluated for expression of maturation markers such as CD86, CD83, CD40, HLA-DR and OX40L by flow cytometry. After 3-4 days of maturation 30-70% of all mature DCs expressed OX40L on the surface. After confirming OX40L expression (FIG. 8), DCs were frozen in freezing medium (90% Fetal Calf Serum+10% DMSO) to be used later in MLR assays

Mixed Lymphocyte Reaction

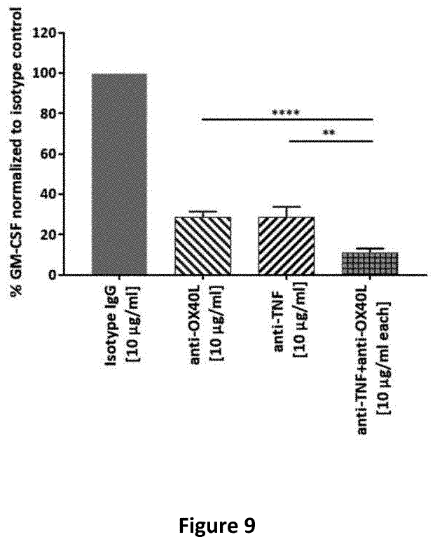

PBMCs were isolated from whole blood or buffy coats via gradient centrifugation. Cells were resuspended in X-Vivo 15 medium (Lonza) and counted. In the meanwhile, DCs were thawed and resuspended in X-Vivo 15 medium. 1×10⁵ PBMCs and 5×10³ DCs were mixed in the same well of a U-bottom 96-well plate. In order to characterize the effect of treatment with anti-TNFα [10 μg/ml] alone, or anti-OX40L alone [10 μg/ml], or a combination of anti-TNFα [10 μg/ml]+anti-OX40L [10 μg/ml], the antibodies where added at the respective dilutions and the mixtures of PBMCs and DCs were incubated for 5 days. Similarly, in order to characterize the effect of treatment with ISVD constructs, varying concentrations of F027300252 (400-0.13 nM; 5-fold serial dilution) or control VHH IRR00119 were added, and the mixture of PBMCs and DCs were incubated for 5 days. Incubation with a control isotype IgG (Biolegend; Clone QA16A12) was used as negative control. After 5 days, the supernatant was collected and the quantities of various cytokines in the supernatant were evaluated by Luminex-based multiplex assays (FIG. 9). For the determination of the anti-TNFα and/or anti-OX40L efficacy DCs from 3 different human donors were tested against PBMCs from 5 allogeneic donors. For determination of the IC50 value for inhibition GM-CSF production by F027300252, PBMCs from 8 donors where tested against DCs derived from 2 allogeneic donors. The XLfit program in Speed was used for fitting the dose response curves and calculating the IC50 values for F027300252.

Treatment with an anti-TNFα antibody alone or an anti-OX40L antibody alone, both at saturating concentrations of 10 μg/ml, did lead to considerable inhibition of GM-CSF secretion compared to treatment with isotype (FIG. 9). However, treatment with the combination of anti-TNFα and anti-OX40L antibodies was able to suppress GM-CSF secretion significantly more potent in comparison to individual TNF (**p<0.0016; FIG. 9) or OX40L (****p<0.0001; FIG. 9) blockade, suggesting superior potency for the combined blockade of TNFα and OX40L (FIG. 9). For determination of the IC50 value for inhibition GM-CSF production by F027300252, PBMCs from 8 donors where tested against DCs derived from 2 allogeneic donors. The XLfit program in Speed was used for fitting the dose response curves and calculating the IC50 values. F027300252 inhibits GM-CSF production in a concentration-dependent manner with an IC50 of 51.69±19.3 nM (SEM) and a maximum inhibition of 70.32±5.59%.

### 6.12 Example 12: Multispecific ISVD Construct Binding to Pre-Existing Antibodies

The binding of pre-existing antibodies, that are present in 96 serum samples from healthy volunteers, to ISVD construct F027300252 was determined using the ProteOn XPR36 (Bio-Rad Laboratories, Inc.). PBS/Tween (phosphate buffered saline, pH7.4, 0.005% Tween20) was used as running buffer and the experiments were performed at 25° C.

ISVD constructs were captured on the chip via binding of the ALB building block to HSA, which is immobilized on the chip. To immobilize HSA, the ligand lanes of a ProteOn GLC Sensor Chip were activated with EDC/NHS (flow rate 30 μl/min) and HSA was injected at 100 μl/ml in ProteOn Acetate buffer pH 4.5 to render immobilization levels of approximately 3200 RU. After immobilization, surfaces were deactivated with ethanolamine HCI (flow rate 30 μl/min).

Subsequently, ISVD constructs were injected for 2 min at 45 μl/min over the HSA surface to render an ISVD construct capture level of approximately 800 RU. The samples containing pre-existing antibodies were centrifuged for 2 minutes at 14,000 rpm and supernatant was diluted 1:10 in PBS-Tween20 (0.005%) before being injected for 2 minutes at 45 μl/min followed by a subsequent 400 seconds dissociation step. After each cycle (i.e., before a new ISVD construct capture and blood sample injection step) the HSA surfaces were regenerated with a 2-minute injection of HCI (100 mM) at 45 μl/min. Sensorgrams showing preexisting antibody binding were obtained after double referencing by subtracting 1) ISVD-HSA dissociation and 2) non-specific binding to reference ligand lane. Binding levels of pre-existing antibodies were determined by setting report points at 125 seconds (5 seconds after end of association). Percentage reduction in pre-existing antibody binding was calculated relative to the binding levels at 125 seconds of a reference ISVD construct.

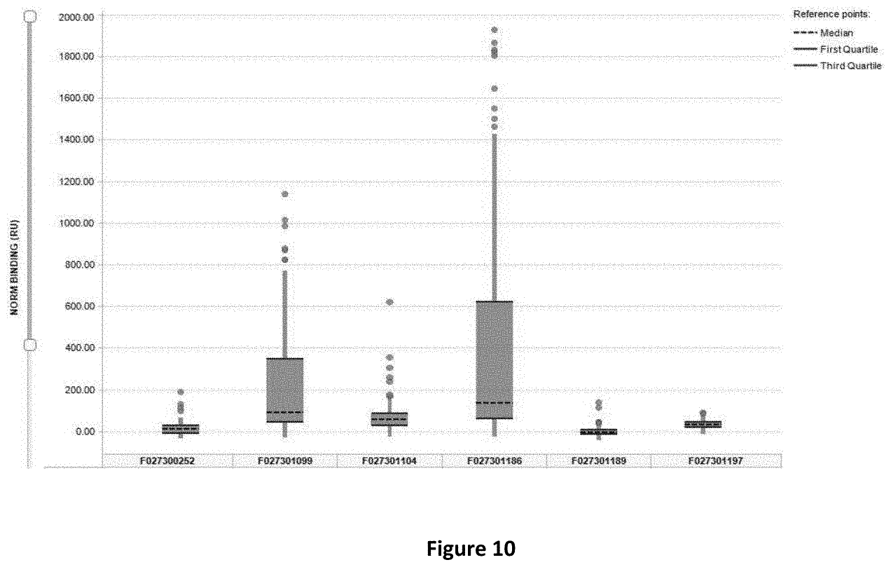

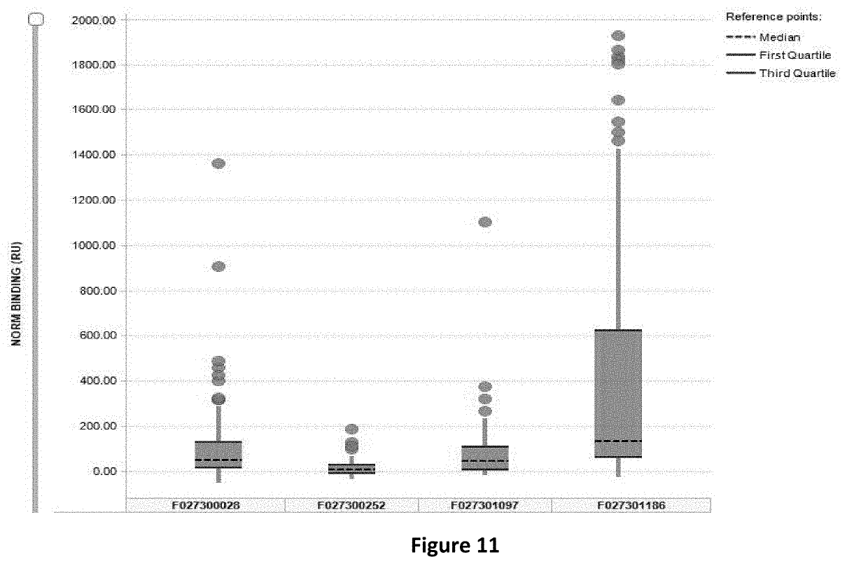

The pentavalent ISVD construct F027300252, optimized for reduced pre-existing antibody binding by introduction of mutations L11V and V89L in each building block and a C-terminal alanine, showed substantially less binding to pre-existing antibodies compared to the control non-optimized pentavalent ISVD construct F027301186 (Table 6, Table 14, FIG. 10 and FIG. 11).

Pre-existing antibody binding depended on the valency and composition of the multispecific constructs. Table 6 and FIG. 10 demonstrate that the pentavalent ISVB construct F027300252 showed lower pre-existing antibody reactivity than tetravalent ISVD constructs F027301104 and F027301099.

Four ISVD constructs, composed of the same parental building blocks as ISVD construct F027300252 displayed different pre-existing antibody reactivities (Table 14, FIG. 11). Comparing ISVD construct F027300028 with ISVD construct F027301186 shows that the introduction of mutations L11V and V89L in each building block and a C-terminal alanine reduced the pre-existing antibody reactivity significantly. Comparing ISVD construct F027300028 with ISVD construct F027301097 shows that the introduction of a T110K mutation in the C-terminal building block slightly further reduced the pre-existing antibody reactivity. The pre-existing antibody reactivity was further reduced significantly by replacing the 35 GS linkers in ISVD construct F027301097 with shorter 9GS linkers in ISVD construct F027300252. Thus, using short 9 GS linkers in multivalent constructs was critical to obtain low pre-existing antibody reactivity.

TABLE 14
Binding of pre-existing antibodies present in 96 human serum samples to ISVD
construct F027500252 compared to three ISVD constructs consisting of
the same building blocks in the same order, but with different linkers or different
building block mutations. L11V +and V89L + refers to the presence of the
respective mutations in all building blocks of the construct. T110K + refers to the presence of this
mutation in the C-terminal building block of the construct. −A = C-terminal alanine present.

| Construct | | linker | | linker | | linker | | linker | | | | | | 25% | Median | 75% |
|---|---|---|---|---|---|---|---|---|---|---|---|---|---|---|---|---|
| ID | BB1 | 1 | BB2 | 2 | BB3 | 3 | BB4 | 4 | BB5 | | L11V | V89L | T110K | percentile | RU level | percentile |
| F027301186 | 1E07/1 | 35GS | 1E07/1 | 35GS | 1C02/1 | 9GS | ALB | 9GS | 1C02/1 | | − | − | − | 61 | 135 | 622 |
| F027300028 | 1E07/1 | 35GS | 1E07/1 | 35GS | 1C02/1 | 9GS | ALB | 9GS | 1C02/1 | −A | + | + | − | 15 | 51 | 128 |
| F027301097 | 1E07/1 | 35GS | 1E07/1 | 35GS | 1C02/1 | 9GS | ALB | 9GS | 1C02/1 | −A | + | + | + | 9 | 46 | 106 |
| F027300252 | 1E07/1 | 9GS | 1E07/1 | 9GS | 1C02/1 | 9GS | ALB | 9GS | 1C02/1 | −A | + | + | + | −8 | 10 | 29 |

### 6.13 Example 13: Evaluation of the Multispecific Anti-TNFα/OX40L ISVD Construct F027300252 in the Chronic Human TNFα Transgenic Tg197 Polyarthritis Model

The F027300252 multispecific anti-TNFα/OX40L ISVD construct was profiled in the Tg197 mouse model of TNF-driven progressive polyarthritis (Keffer at al., 1991, EMBO J., 10:4025-4031). In these mice, a modified human TNFα gene was inserted as a transgene into mice. The human gene was modified in a way to render the transcribed mRNA more stable, and thus led to overexpression of TNFα and a spontaneous progressive arthritis in all four paws at 100% penetrance. Signs and symptoms became visible at about 6 weeks of age and were constantly increasing until they led to significant moribundity and mortality from about 10 weeks of age onwards if left untreated. Arthritis severity was clinically assessed by a scoring system as detailed below:

| ARTHRITIS SCORE¹ | CHARACTERISTICS |
|---|---|
| 0/no disease | no arthritis (normal appearance, mouse |
| | can support its weight clinging to an inverted or |
| | tilted surface such as a wire grid or a |
| | cage lid for a period of time, whole body |
| | flexibility/evasiveness normal, |
| | grip strength maximum) |
| 0.5/mild disease | onset of arthritis (mild joint swelling, |
| | all other parameters as above) |
| 1/mild to | mild to moderate (joint distortion by |
| moderate | swelling, inflamed paw, all other parameters as |
| disease | above) |
| 1.5/moderate | moderate arthritis (joint-paw swelling, |
| disease | distortion + last finger inward deformation, brief |
| | support clinging to an inverted or tilted |
| | surface such as a wire grid or a cage lid, whole |
| | body flexibility reduced, reduced grip strength) |
| 2/moderate | moderate to severe arthritis (severe joint, |
| to severe | paw and finger swelling, joint-leg |
| disease | deformation, no support clinging to an inverted |
| | or tilted surface such as a wire grid or a |
| | cage lid, no whole-body flexibility, no grip |
| | strength, climbing/feeding affected, starts |
| | shaking when trying to move, but manages |
| | to move forward) |
| 2.5/severe | severe arthritis (as above 2 + finger |
| disease | deformation in front paws, mouse movement |
| | impaired, shaking not willing to move) |
| 3/very severe | very severe arthritis (ankylosis detected |
| disease | on flexion and severely impaired movement, |
| | mouse moribund, not shaking anymore, cannot |
| | turn/flip around readily when tilted to the |
| | side). |

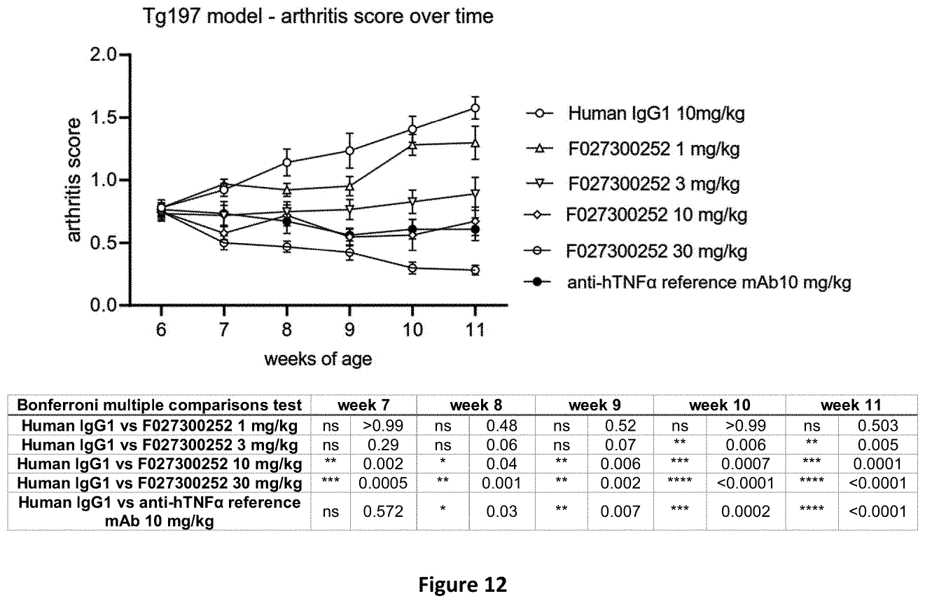

¹Arthritis score as indicated on the y-axis in FIG. 12.

Arthritis was sensitive to treatment with therapeutic agents directed towards inhibition of human TNFα (Shealy et al., 2002, Arthritis Res. 4(5): R7).

For the purpose of establishing dose-dependent efficacy, different doses of the ISVD construct were administered by twice weekly intraperitoneal injection in a therapeutic manner to animals of 6 weeks of age with visible signs and symptoms of arthritis (n=8 animals per group). Human IgG1 purified from human myeloma serum (BioXcell #BE0297) was used as negative control, and an anti-hTNFα reference mAb was used as positive control to suppress arthritis. The F027300252 ISVD construct was administered at four different dose strengths of 1 mg/kg of body weight, 3 mg/kg, 10 mg/kg, and 30 mg/kg, respectively. Treatment was continued until 11 weeks of age. Clinical arthritis scores were determined once per week. As shown in FIG. 12, ISVD construct treatment resulted in a dose-dependent suppression of clinical arthritis scores over time.

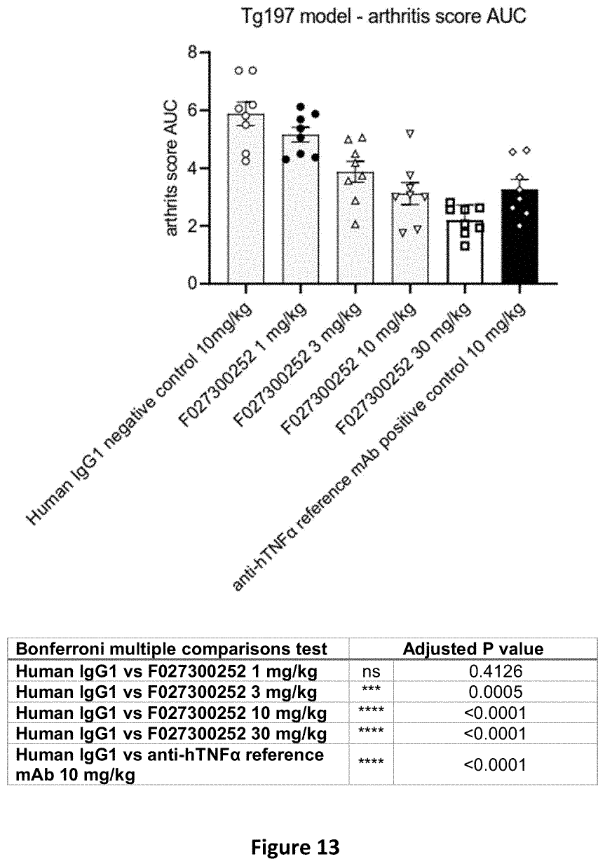

Animals treated with human IgG1 negative control antibody develop a mean arthritis score of 1.58±0.06 by week 11. Anti-hTNFα reference mAb fully suppressed arthritis progression, with a mean score of 0.61±0.06 by week 11. F027300252 reduced the arthritis progression to week 11 mean scores of 1.30±0.09 (1 mg/kg), 0.89±0.10 (3 mg/kg), 0.67±0.08 (10 mg/kg), and 0.28±0.04 (30 mg/kg). Overall suppression of arthritis was analyzed by Area under the curve (AUC, FIG. 13). All doses of F027300252 significantly suppressed arthritis progression comparable to anti-hTNFα reference mAb in the Tg197 arthritis model.

Upon completion of treatment, hindlimb ankle joints were processed for histology and section were evaluated for structural signs of arthritis with the following scoring system:

CUMULATIVE HISTOPATHOLOGICAL CRITERIA FOR
SCORING ARTHRITIC PHENOTYPE IN THE ANKLE JOINTS

| SCORE¹ | DISEASE | CRITERIA |
|---|---|---|
| 0 | Normal | no detectable pathology |
| 1 | Mild | hyperplasia of the synovial membrane |
| | | and presence of polymorphonuclear |
| | | infiltrates. Mild tendonitis may be present. |
| 2 | Moderate | pannus and fibrous tissue formation |
| | | and focal subchondrial bone erosion |
| 3 | Moderate- | cartilage destruction and bone erosion |
| | | Severe |
| 4 | Severe | extensive cartilage destruction and bone |
| | | erosion. Bone outline structure is lost |

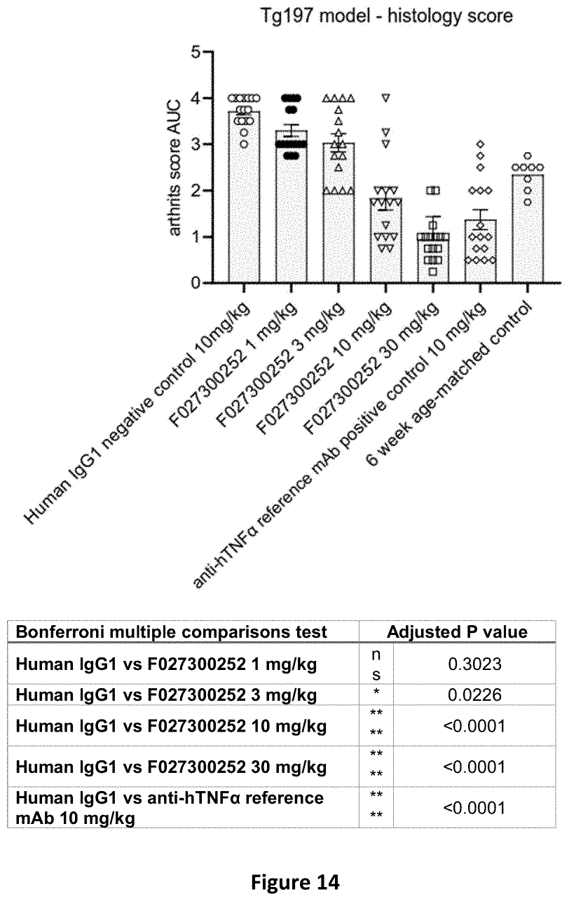

¹Arthritis score as indicated on the y-axis in FIG. 14.

The results of the histology scoring are depicted in FIG. 14. Structural arthritis and joint destruction were significantly suppressed by F027300252 at higher doses.

In conclusion, the results demonstrate dose dependent suppression of arthritis signs and symptoms as well as inhibition of structural progression by the ISVD construct F027300252 to an extent comparable with anti-hTNFα reference mAb.

### 6.14 Example 14: Evaluation of the Anti-TNF/OX40L ISVD Construct in a Human TNFα Driven Acute Rheumatoid Arthritis Mouse Model (CAIA)

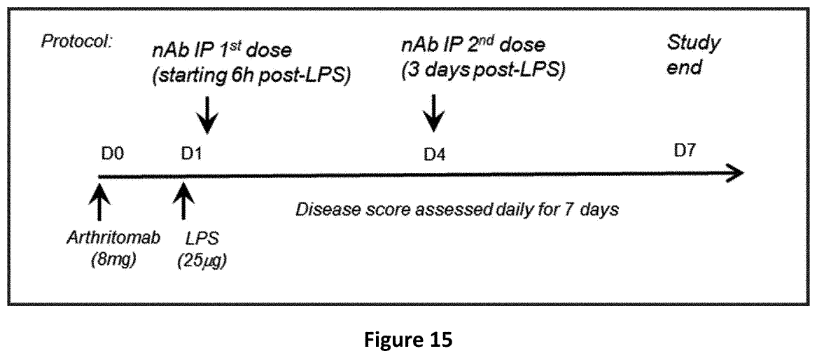

The in vivo efficacy of the anti TNF-OX40L ISVD construct (F027300252) was evaluated in an acute rheumatoid arthritis model called collagen-antibody induced arthritis (CAIA). The CAIA is a pre-clinical model of rheumatoid arthritis and is widely used to assess anti-arthritic drug effects in drug development (Nandakumar & Holmdahl (2007), Methods Mol Med.; 136:215-23). It is a shorter term (7 days) induced arthritis model with a cocktail of monoclonal anti-collagen II antibodies and LPS. A humanized TNFα and TNFR1 mouse was used for this experiment: C57BL/6NTac-Tnfrsf1a^{tm4504.1(TNFRSF1A)Tac}Tnf^{tm4503.1(TNF)Tac}. All animals were dosed and monitored according to guidelines from the Institutional Animal Care and Use Committee on study protocols approved by the Laboratory Animal Welfare Committee at Sanofi under the license from the German animal welfare government agency. In vivo arthritis scores were assessed in an operator-blinded fashion. Male and female mice at the age of a minimum of 10 weeks were equally randomized to the respective treatment groups. Mice received 8 mg of a cocktail of monoclonal anti-collagen antibodies (ArthritoMab, MDbiosciense, CIA-MAB-2C) by intraperitoneal (ip) injection in sterile PBS on day 0, followed by 25 μg LPS IP in PBS 24 hours later. Mice were monitored for 7 days. Treatment was administered 6 hours after LPS on day 1 with Isotype control (IgG1 Isotype 1.0 mg/kg i.p. 200 μl/mouse), multispecific TNFα-OX40L ISVD construct F027300252 at 0.03, 0.1, 0.3, 1 mg/kg (200 μl/mouse) compared to anti-hTNFα reference mAb (conventional antibody) at 0.1 and 0.5 mg/kg in 200 μL/mouse. The dose applied equals a molar exposure estimated at 0.45, 1.5, 4.5, and 15 nmol/kg for F027300252 and 0.65 and 3.3 nmol/kg for anti-hTNFα reference mAb. For anti-hTNFα reference mAb two studies were conducted, and the vehicle animals were pooled for the final analysis. A second dose was applied to all animals three days after the first dose on day 4 of the experiment. A schematic study design of the experiment is depicted in FIG. 15.

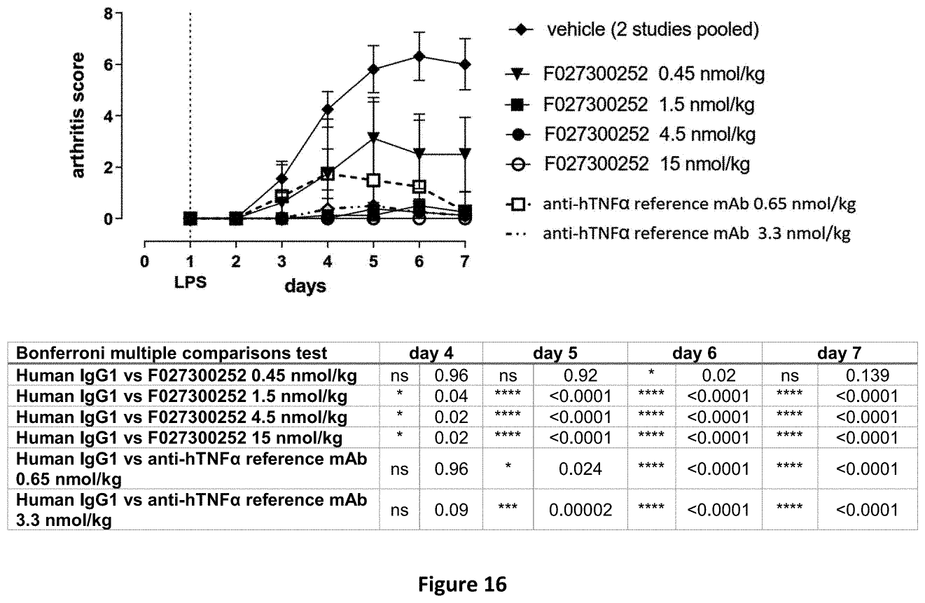

The results of the experiments are shown in FIG. 16. The vehicle-treated control animals were pooled from two experiments with different anti-hTNFα reference mAb concentrations tested vs. control. A mean peak increase in arthritis score of 6.135 was achieved on day 6. The positive control anti-hTNFα reference mAbshowed a pronounced effect on the arthritis score and completely blocked disease development at the higher concentration tested (3.3 nmol/kg). The anti-TNFα-OX40L ISVD construct F027300252 demonstrated a dose-dependent effect with similar in vivo potency compared to anti-hTNFα reference mAb. Only the lowest dose of 0.45 nmol/kg showed approximately 50% effect which is comparable to the lower dose anti-hTNFα reference mAb with 0.65 nmol/kg but all other doses tested for the ISVD construct completely blocked disease development. The second lowest dose tested with 1.5 nmol/kg for F027300252 seems to be at least similar if not more effective than anti-hTNFα reference mAb at 3.3 nmol/kg.

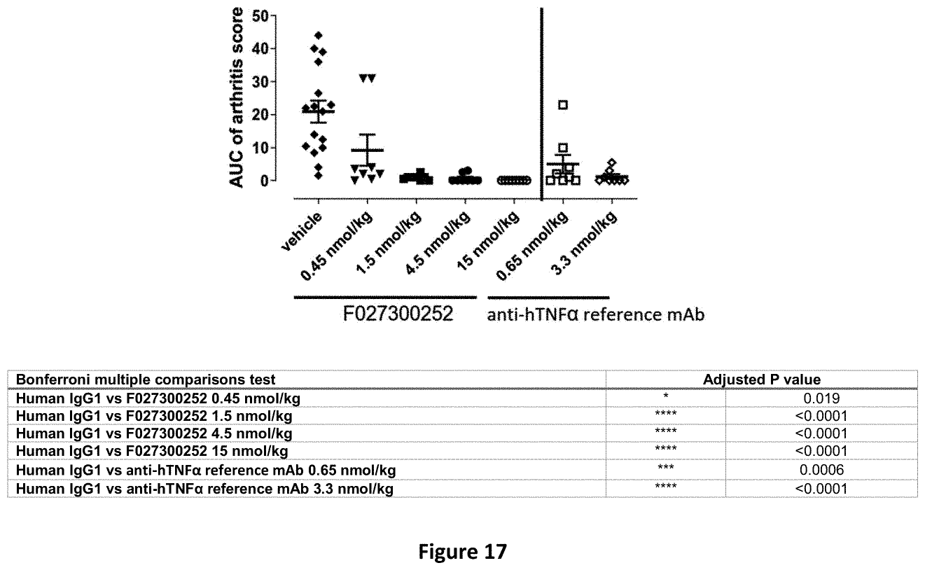

The pronounced dose-dependent effect of the ISVD construct F027300252 is more obvious in FIG. 17, where the AUC of the arthritis score data in FIG. 16 is depicted. All doses of F027300252 significantly reduced the arthritis score with complete disease inhibition from 1.5 nmol/kg upwards. The effect was comparable to anti-hTNFα reference mAb at equimolar exposure/dose.

In conclusion, these results demonstrate that the anti-TNF-OX40L ISVD construct was as good or potentially superior in regard to targeting human TNFα compared to anti-hTNFα reference mAb in an acute rheumatoid arthritis model in mice which highlights its immunosuppressant potential for the treatment of auto-immune diseases such as rheumatoid arthritis. Statistics are 1-way ANOVA and Bonferroni multiple comparison test.

### 6.15 Example 15: Proof of Mechanism in a Combined Non-Human Primate T-Cell Dependent Antibody Response (TDAR) and Delayed Type Hypersensitivity (DTH) Model with F027300252

The T-cell dependent antibody response (TDAR) model is a measure of immune function that is dependent upon the effectiveness of multiple immune processes, including antigen uptake and presentation, T cell help, B cell activation, and antibody production. In this study we used it to determine the pharmacodynamic effect of anti TNF-OX40L ISVD construct F027300252 in vivo in non-human primates. The objective of this study on top of pharmacodynamics was to determine PK and safety of F027300252 following 5 weekly subcutaneous administrations to female cynomolgus monkeys followed by a 30 days period without treatment after the last dosing of F027200252 on day 29. In order to assess the effects of F027300252 on the immune system functionalities, the humoral response was evaluated in-life through a TDAR assay (after Keyhole Limpet hemocyanin—KLH—immunization), while the cellular immune response was evaluated through the in-vivo delayed-type hypersensitivity (DTH) test and ELISPOT ex vivo assays using the same antigen as for the TDAR, KLH.

For this study a total of 20 female purpose-bred cynomolgus monkeys (Macaca fascicularis) were used. We selected non-human primates as a species because of the highly selective binding of F027300252 to the human targets. Non-human primates were chosen based on the high cross-reactivity to cynomolgus monkey for F027300252. The test item is not cross-reactive to other rodent or non-rodent species. Hence, based on available data the cynomolgus monkey was selected as the non-rodent species for pharmacology and non-clinical safety testing and background data from previous studies are available at the contract research organization (CRO) we worked with, Citoxlab, France. Additionally, the TDAR and DTH assays have been validated in cynomolgus monkeys before at the CRO we worked with.

Preliminary safety of F027300252 was assessed in a repeat dose cyno study at 25 mg/kg by the subcutaneous (sc) route with two administrations separated by 2 weeks (study no. DIV1953). The doses selected in this combined TDAR-DTH study span over a range that covers both potentially pharmacological doses (3 and 10 mg/kg) and also higher doses for safety assessment (30 and 100 mg/kg). The dose formulations were administered weekly for a period of 29 days with a total of 5 administrations. Day 1 corresponds to the first day of the treatment period with the test and control items.

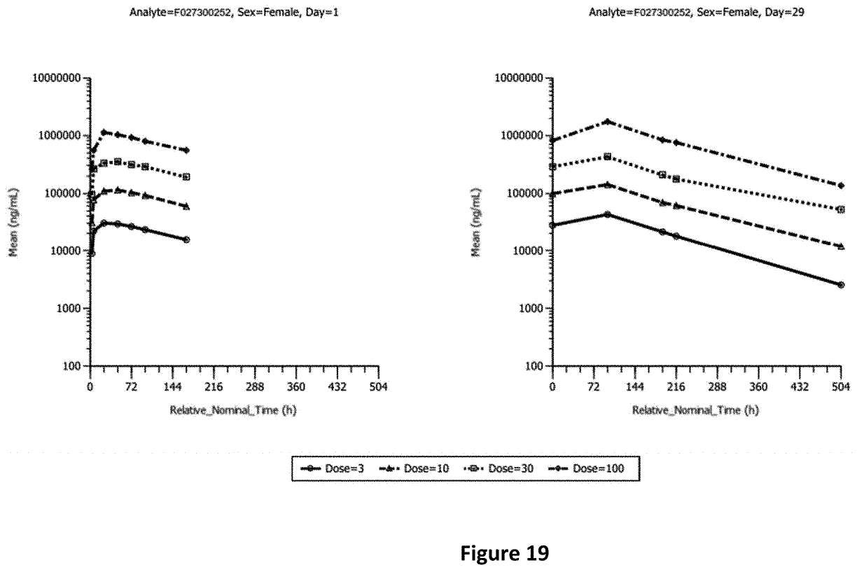

The pharmacokinetics of the selected doses is depicted in FIG. 19.

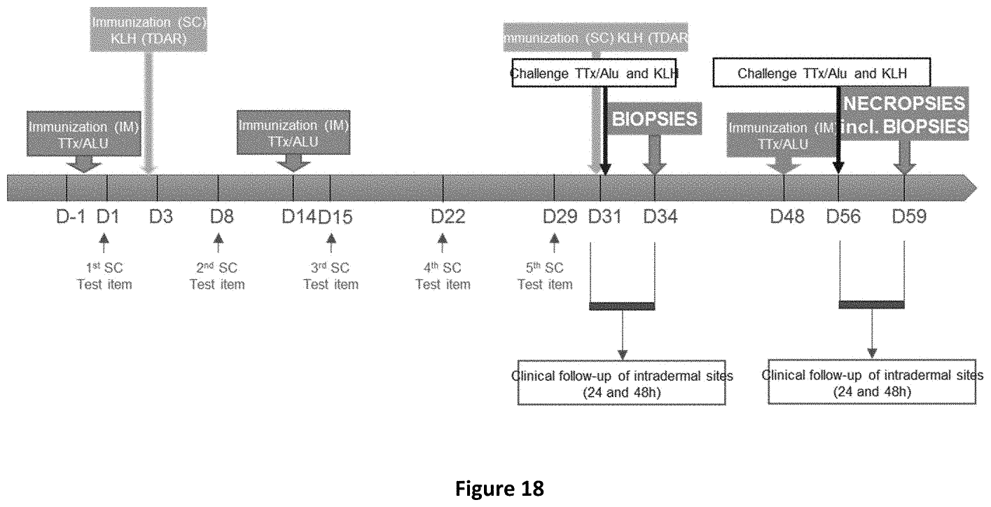

The KLH antigen was administered on day 3 and day 31 subcutaneously at a dose of 10 mg/animal in 1 ml (FIG. 18). We used Imject® Mariculture Keyhole Limpet Hemocyanin (ThermoFisher Scientific, ref. No. 77600). For the quantification of anti-KLH IgG venous blood (1 mL) was collected from an appropriate vein into a plain tube according to a pre-defined planning schedule. The blood was kept at room temperature to allow clotting (maximum 2 hours) pending centrifugation (approximately 3000g for 10 minutes at +4° C.), and the resulting serum was split into 3 aliquots of 80 μL+1 aliquot of the remaining volume. The tubes were kept frozen at −20° C. until analysis. Anti-KLH IgG levels were measured using a specific ELISA method developed and validated at Citoxlab France (Citoxlab France/Study Nos. 41106 RDP for anti KLH IgG). For each of the two periods (Day 3 [prior to KLH injection] to Day 31 [prior to KLH injection] and Day 31 [prior to KLH injection] to Day 59), the area under the curve (AUC) was calculated for each animal. AUC values were transformed in logarithm (log 10(value+1)), and analyzed by period using a Wilcoxon test.

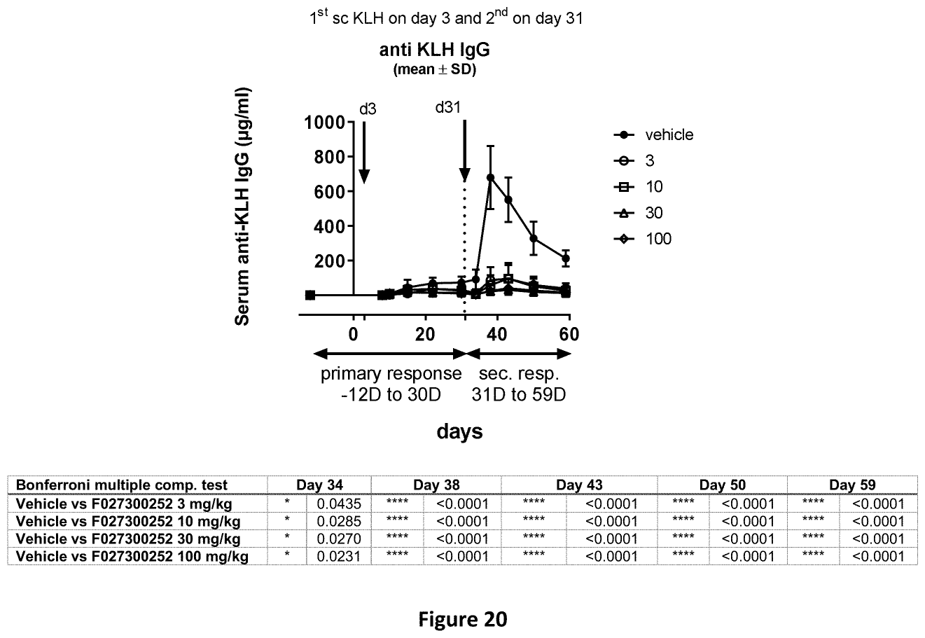

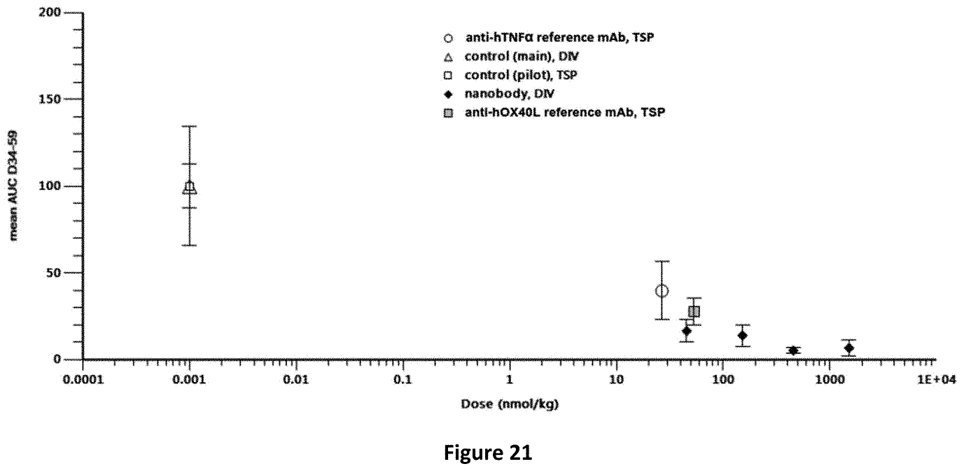

As expected, the primary response to KLH with IgG was minor. After the second exposure to KLH a strong anti KLH IgG response was elicited in the vehicle control treated group. Treatment with F027300252 resulted in a strong inhibition of this response from the lowest dose tested upwards (FIG. 20). In comparison to data from a pilot study using tool monoclonal antibodies against TNFα (anti-hTNFα reference mAb) and OX40L (anti-hOX40L reference mAb), the AUC of the F027300252 study was plotted (FIG. 21). Even at the lowest dose tested for F027300252 (referred to as “nanobody” in FIG. 21) (3 mg/kg) we observed a more pronounced reduction in AUC compared to anti-hOX40L reference mAb and anti-hTNFα reference mAb mono treatment.

At necropsy we harvested Peripheral Blood Mononuclear Cells (PBMCs) from the monkeys for an ex vivo re-stimulation using the same antigen (KLH) in an Enzyme-linked immunospot (ELISPOT) assay to measure cellular immune response. The ELISPOT assay is a sensitive immunoassay that measures the frequency of cytokine-secreting cells at the single-cell level. In this assay, cells were seeded into the wells of a 96-well plate pre-coated with a capture antibody specific to the cytokine being assayed (IFN-γ and IL-4 in this case). Cytokines that are secreted by the cells in the presence (or absence) of stimuli, were captured by the specific antibodies on the surface of well bottom in the vicinity of the secreting cell. After an appropriate incubation time, cells were removed, and the secreted cytokine was visualized using a biotinylated detection antibody. After several washing steps, an enzyme (alkaline phosphatase) coupled with Streptavidin was added. By using a precipitating substrate, the immobilized cytokine was then revealed as an ImmunoSpot (i.e. individual cytokine-secreting cell). On each PBMC sample, the frequency of IFN-γ and IL-4 secreting cells was analyzed after the stimulation with KLH.

ELISPOT plates were numerically scanned at the test site using an Immunospot® ELISPOT analyzer (images of individual wells taken by the instrument). The images were then analyzed on a dedicated software for spot count evaluation. The presence of spots and number of spots was evaluated in each well and corresponding results expressed as the number of IFN-γ or IL-4 spot forming cells (sfc) were calculated. Number of sfc were normalized per million of PBMC.

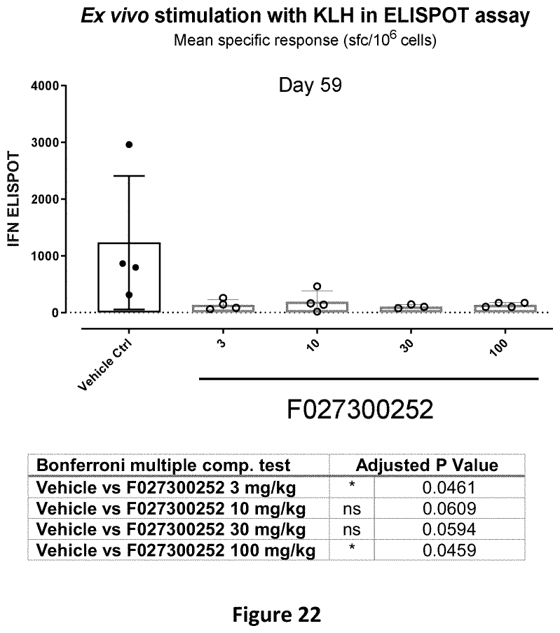

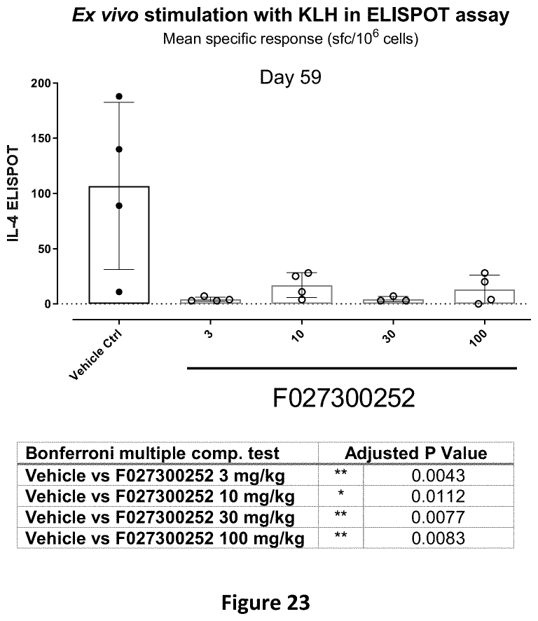

Cells stimulated from vehicle control animals showed a marked increase in either IFN-γ (FIG. 22) and IL-4 (FIG. 23). Upon treatment both cytokines were markedly decreased in all doses tested for F027300252 (FIGS. 22 and 23).

In conclusion, these results demonstrate that the anti-TNF-OX40L ISVD construct strongly inhibited the interaction between antigen-presenting cells, T-cells, B-cells in a T-cell dependent antibody response proof of mechanism model conducted in non-human primates.

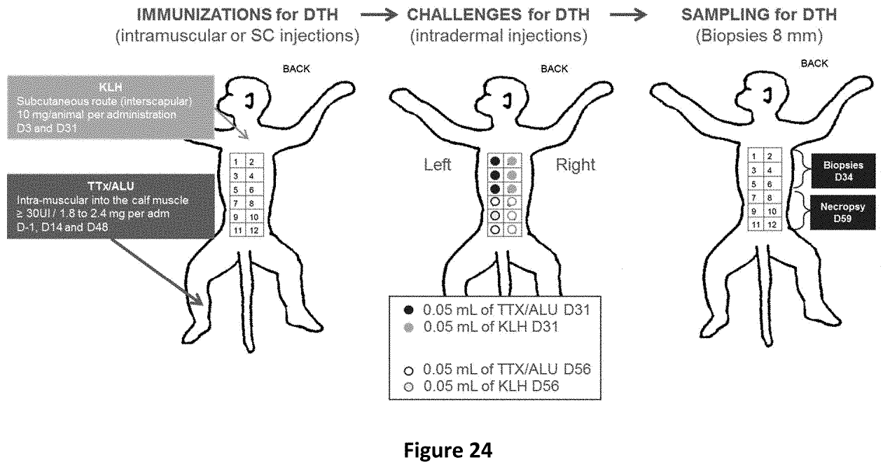

The second part of the non-human primate study focused on an in vivo delayed type hypersensitivity (DTH) readout in the skin to assess the cellular immune response in-life. Tetanus Toxoid (TTx) and Aluminum hydroxide were used as antigen. The DTH challenge was applied as described in FIG. 18. A grid of approximately 21 cm length×3 cm width (i.e. with squares of 3 cm side) was delimited on each side of the back (FIG. 24) with an indelible chirurgical pen on the skin, with a point indicating the injection site in the center of each square. The injection site was disinfected with chlorhexidine gluconate solution (Antisept® Spray).

The antigens (TTx/ALU and KLH) were each injected in the center of six squares on the day of injection. Intradermal injections were performed using a single use sterile plastic syringe fitted with a sterile single use 29 G needle, by stretching the skin and introducing the needle in the thickness of the skin (bevel up). A little vesicle appeared at the injection site. Then, the needle was quickly removed from the skin.

During the in-life phase of the DTH the following parameters were assessed and documented (table 15).

TABLE 15

| Erythema formation | |
|---|---|
| no erythema | 0 |
| very slight erythema (barely perceptible) | 1 |
| well-defined erythema | 2 |
| moderate to severe erythema | 3 |
| severe erythema (beet redness) to slight | 4 |
| eschar formation (injuries in depth) | |
| Edema formation | |
| no edema | 0 |
| very slight edema (barely perceptible) | 1 |
| slight edema (edges of area well-defined | 2 |
| by definite raising) | |
| moderate edema (raised approximately 1 millimeter) | 3 |
| severe edema (raised more than 1 millimeter and | |
| extending beyond area of exposure) | 4 |

None of the above listed parameters showed a clear trend for a treatment effect with F027300252 indicating that a histopathological assessment and immunohistochemistry might be more sensitive to demonstrate a difference compared to the skin macroscopic alterations during the in-life phase of the DTH part of the model.

For lmmunohistochemistry the following markers were evaluated: CD3 (T lymphocytes), CD4 (T helper cells), CD8 (cytotoxic T cells), CD30 (B lymphocytes), CD68 (macrophages), Ki67 (proliferation marker), and FoxP3 (T regulatory cells). The results of conventional H&E histopathology and immunohistochemistry (IHC) staining are summarized below.

For the first antigen, TTx/ALU, on day 34 the immune response was dominated by macrophages. A slight dose-dependent decrease in the severity of the inflammation with most pronounced effects at highest dose was observed. lmmunohistochemistry revealed a remarkable decrease in the score of all markers in the highest dose tested with mainly macrophages being decreased. At necropsy on day 59, an overall slightly lower inflammatory response was observed compared to day 34. Similar to day 34, IHC revealed the most prominent effect on the proliferation marker Ki67. Overall, the effect was most pronounced at the highest dose tested for F027300252.

For the second antigen tested, KLH, observation on day 34 showed an immune response that was dominated by eosinophil granulocytes. Overall, the inflammatory response was moderately lower compared to TTX/Alu on day 34. IHC revealed a remarkable decrease in the score of all markers in the highest dose tested with CD8+, CD20, and Ki67 mainly decreased. On day 59 the inflammatory response was again moderately lower compared to TTX/Alu on day 59 and minimally lower compared to KLH on day 34. It was a minimal decrease with a clear dose-response in the severity of the inflammation observed. Similar to day 34, IHC revealed the most prominent effect at the highest dose tested of F027300252 on CD3, CD8+, CD20, and FoxP3 which were mainly decreased.

Overall, the described data provide a proof of mechanism in a combined non-human primate T-cell dependent antibody response (TDAR) and delayed type hypersensitivity (DTH) model with F027300252, a novel multispecific ISVD construct targeting TNFα and OX40L which was confirmed with a KLH induced ex vivo ELISPOT assay measuring IFN-γ and IL4 release.

### 6.16 Example 16: Evaluation of the Anti-TNF/OX40L ISVD Construct F027300252 in the Xenogeneic Graft-Versus-Host Disease Model

In vivo efficacy of the anti-TNFα/OX40L ISVD construct F027300252 was evaluated in a model of xenogeneic graft-versus-host disease (xeno-GVHD). In this model, human peripheral blood mononuclear cells (hPBMCs) are injected into irradiated, immunocompromised NOD-scid IL2rgamma(null) (NSG) mice (King et al. (2009), Clin Exp Immunol., 157(1):104-118). Engrafted hPBMCs attack the murine host in a major histocompatibility complex-dependent manner, leading to symptoms of acute GVHD (Brehm et al. (2019), FASEB I., 33(3):3137-3151).

Female NSG mice at the minimum age of 6 weeks were randomized equally to the respective treatment groups. Mice were irradiated with 1 Gγ one day before intravenous (iv) injection with 2×10′ hPBMCs. Animals were individually scored in an operator-blinded fashion three times a week using following scoring system (Riesner et al. (2016), Bone Marrow Transplant., 51(3):410-417):

TABLE 16

| Score | Posture | Fur | Activity | Skin | Weight loss |
|---|---|---|---|---|---|
| 0.5 | mild kyphosis, | mild ruffling | mild decreased | reddish, irritated | >5% |
| | only at rest | | | areas | |
| 1 | moderate | moderate | moderate | scaling, dry skin | >10% |
| | kyphosis | ruffling (>20%) | decreased, | areas (<20%) | |
| | | | isolation | | |
| 1.5 | strong | severe ruffling | stationary until | scaling, dry skin | >15% |
| | kyphosis, | (>40%), mild | stimulated, | areas (>20%) | |
| | slightly impaired | loss of fur | coordination | | |
| | movement | | disorders, | | |
| | | | stereotypes | | |
| 2 = abort | severe | severe ruffling | lethargy, | scaling, dry skin | >20% |
| | kyphosis, | (>60%), poor | paralysis, | areas (>40 %), | |
| | distinct | grooming, | staggering | open, bloody | |
| | impaired | moderate to | | skin areas | |
| | movement | severe loss of | | | |
| | | fur | | | |

The GVHD score was determined by summation of these parameters. Animals were sacrificed when reaching a single score of 2 or exceeding a cumulative score of 6. The degree of hPBMC engraftment in host mice was assessed by determining human CD45+cells among all CD45+cells in the peripheral blood of host mice using flow cytometry. Bi-specific anti-TNF/OX40L Nanobody F027300252 (10 mg/kg) was administered IP three times a week, starting on day 1 and compared to Isotype treated control animals. All animals were dosed and monitored according to guidelines from the Institutional Animal Care and Use Committee on study protocols approved by the Laboratory Animal Welfare Committee at Sanofi under the license from the German animal welfare government agency.

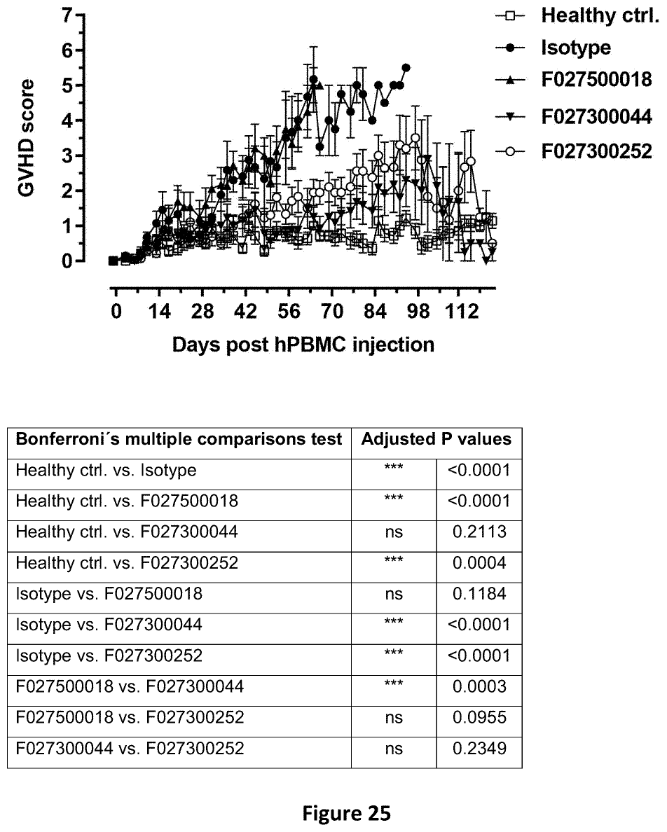

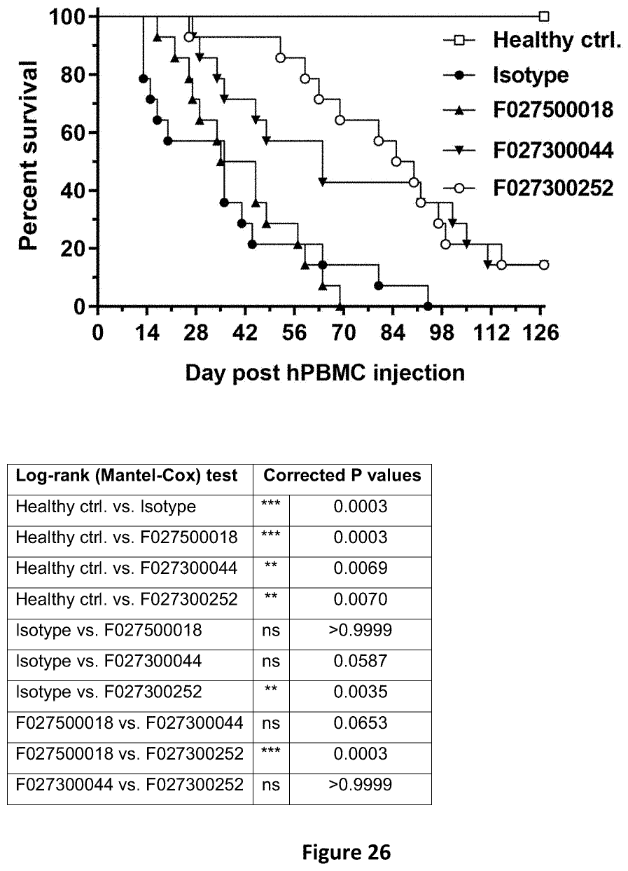

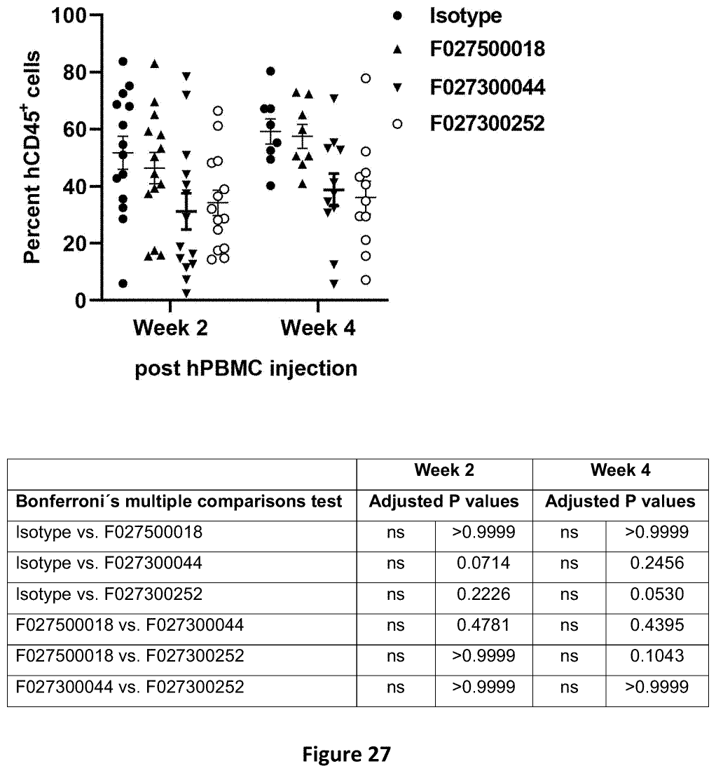

To validate TNF and OX40L as targets for ISVD constructs in the xeno-GVHD mouse model, 150 nmol/kg anti-humanTNF ISVD F027500018, 150 nmol/kg anti-humanOX40L ISVD F027300044 or the bi-specific anti-TNF/OX40L ISVD F027300252 (150 nmol/kg) were administered IP three times a week, starting on day 1 and compared to isotype treated control animals. First symptoms of GVHD in host mice were observed within two weeks after hPBMC injection. In Isotype treated control animals, the GVHD score continuously increased with progression of the studies until all mice of this group were either found dead or sacrificed when the above described humane endpoints were reached. Blockade of TNF had only a mild effect on disease development whereas OX40L blockade was able to significantly ameliorate disease progression (FIG. 25). Dual-targeting by treatment with F027300252 resulted in similar disease development as was observed for OX40L blockade alone. Consequently, survival of F027500018 treated groups was only slightly prolonged whereas F027300044 alone or anti-TNF/OX40L combination treatment with F027300252 resulted in further extended survival (FIG. 26). In addition, the blockade of OX40L using either F02730044 or F027300252 tended to inhibit engraftment of human CD45+cells in host mice (FIG. 27).

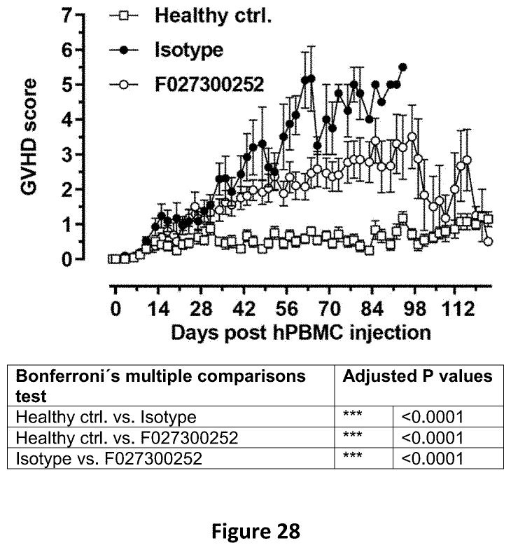

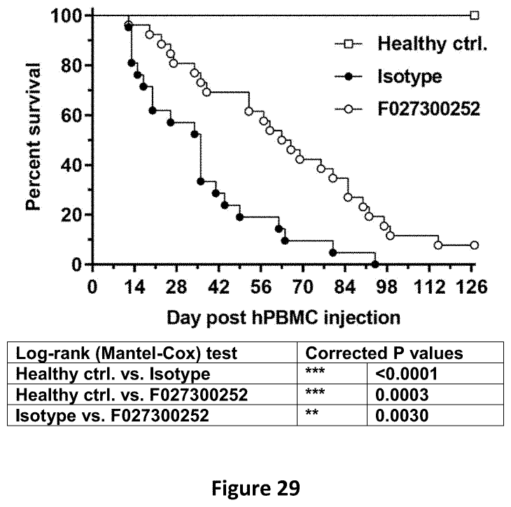

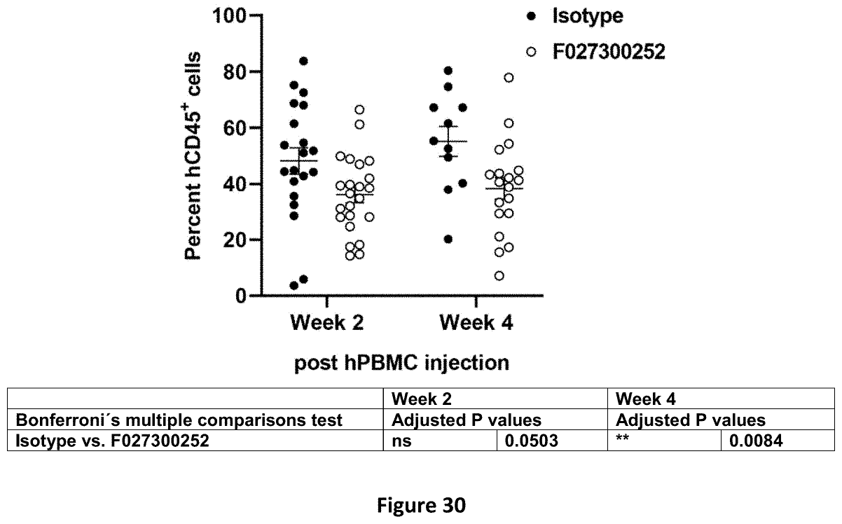

To evaluate the bispecific anti-TNF/OX40L ISVD F027300252 in the xeno-GVHD mouse model, additional data were collected, and results were pooled from two independent studies. GVHD onset was observed within few days in hPBMC transferred mice. 50% of animals that received only isotype ISVD were found dead or reached abort criteria within five weeks post transfer. Even though F027300252 treatment did not prevent disease onset, it was able to ameliorate disease progression (FIG. 28) and consequently prolonged survival of F027300252 treated mice. More than 50% of animals remained alive beyond week 9 (FIG. 29). In some cases, mice treated with F027300252 were able to recover from the disease and survive until the end of the studies. Moreover, the application of F027300252 was found to inhibit engraftment of human CD45+cells in host NSG mice (FIG. 30), which correlates with slower disease development and prolonged survival.

In summary, these results demonstrate efficacy of OX40L blockade and F027300252 treatment in the xeno-GVHD mouse model.

### 6.17 Example 17: Inhibition of the PHA Induced IL-8 Release in Human Whole Blood by an Anti-TNF Antibody and the Bispecific Anti-TNFα/Anti-OX40L ISVD Constructs F027300252, F027301104, F027301189, F027301197, and F027301199

For measuring the effect of the anti-TNFα monoclonal antibody (mAb) RA14956298 (from Sanofi) as well as the bispecific anti-TNFα/anti-OX40L ISVDs F027300252, F027301104, F027301189, F027301197, and F027301199 (all from Sanofi) on the inhibition of PHA-induced IL-8 release in human whole blood, blood from healthy human donors was drawn into vacutainer blood collection tubes (BD #368480) in the presence of Na-Heparin [17 IU/ml] as anti-coagulant. PHA-L (Phytohaemagglutinin-L; from Merck Millipore; ordering number #M5030) was reconstituted as stock solution [1 mg/ml] in sterile water, and a working solution with [50 μg/ml] PHA-L was prepared. Working solutions of the negative control antibody RA11944493 (Sanofi; IgG1 isotype ctrl), the positive control antibody RA14956298 (from Sanofi, the negative control V_{HH} IRR00119 (from Sanofi/Ablynx), and the bispecific anti-TNFα/anti-OX40L ISVD constructs F027300252, F027301104, F027301189, F027301197, and F027301199 (all from Sanofi/Ablynx) were prepared at 500 nM in PBS.

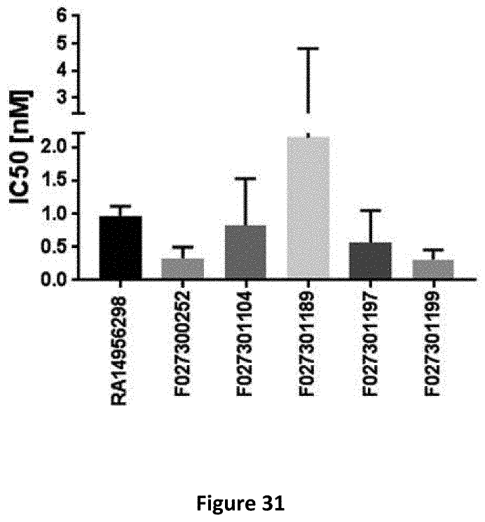

Serial dilutions of the antibodies and ISVD constructs between 8 pM to 25 nM final concentrations in medium [RPMI-1640 (from Gibco; ordering number 61870-010)+10% human AB-serum (from Sigma; ordering number H3667)+1% PenStrep (from Gibco; ordering number 15140-122)] were added in 25 μL to 96we11 microplates (V-bottom, PP; from Eppendorf; ordering number 0030601300). 200 μL of the human blood was added to each well and incubate for 30 min at room temperature with a lid on the plate. PHA-L was diluted to a concentration of 50 μg/ml in medium [RPMI-1640+10% human AB-serum+1% PenStrep], and 25 μl of this PHA-L in medium was added to each well of the pre-incubation mixture of human blood with the antibodies or ISVD constructs in the 96 well plate. Samples were gently mixed, the plates were sealed with a sterile lid (using the Thermo Scientific plate sealer; ordering number 236366), and plates were incubated for 6 h at 37° C., 5% CO2, 95% rH. After incubation the blood samples were centrifuged for 12 min at 2000×g, using middle ramp for acceleration and break. The plasma supernatant was harvested and stored at −80° C. in a new 96 well microplate for further analyses by ELISA. Levels of IL-8 were determined using the Enzyme-Linked ImmunoSorbent Assay (ELISA; from Invitrogen; catalog number 88-8086) for quantitative detection of human IL-8 according to the protocol provided by the manufacturer. The XLfit program in Speed was used for fitting the dose response curves and calculating the IC50 values in FIG. 31. based on the results from 3 different blood donors.

Incubation of human whole blood with the negative control antibody RA11944493 or the negative control V_{HH} IRR00119 did not lead to any inhibition of PHA-induced IL-8 release (data not shown). In contrast, incubation of human whole blood with the monospecific anti-TNF monoclonal antibody (mAB) RA14956298 led to a strong inhibition of PHA-induced IL-8 release with an IC50 of 0.96 nM (±0.08 SEM) (FIG. 31). Incubation of human whole blood with the bispecific anti-TNFα/anti-OX40L ISVD construct F027300252 led to an even stronger inhibition of PHA-induced IL-8 release with an IC50 of 0.33 nM (±0.09 SEM) (FIG. 31). The bispecific anti-TNFα/anti-OX40L ISVD constructs F027301104, F027301197, and F027301199 also led to a strong inhibition of PHA-induced IL-8 release with IC50 values of 0.8233 (±0.4 SEM), 0.5667 (±0.27 SEM), and 0.3133 (±0.08 SEM), respectively (FIG. 31) The bispecific anti-TNFα/anti-OX40L ISVD construct F027301189 had the lowest potency with an IC50 of 2.143 (±1.54 SEM) (FIG. 31).

### 7 INDUSTRIAL APPLICABILITY

The polypeptides, nucleic acid molecules encoding the same, vectors comprising the nucleic acids and compositions described herein may be used for example in the treatment of subjects suffering from autoimmune or inflammatory diseases.

1. A polypeptide, a composition comprising the polypeptide, or a composition comprising a nucleic acid comprising a nucleotide sequence that encodes the polypeptide, wherein said polypeptide comprises or consists of at least four immunoglobulin single variable domains (ISVDs), wherein each of said ISVDs comprises three complementarity determining regions (CDR1 to CDR3, respectively), optionally linked via one or more peptidic linkers; and wherein:
a. a first ISVD and a second ISVD, each specifically binds to OX40L and each comprises
i. a CDR1 comprising the amino acid sequence of SEQ ID NO: 7 or has 2 or 1 amino acid difference(s) with SEQ ID NO: 7;
ii. a CDR2 comprising the amino acid sequence of SEQ ID NO: 10 or has 2 or 1 amino acid difference(s) with SEQ ID NO: 10; and
iii. a CDR3 comprising the amino acid sequence of SEQ ID NO: 13 or has 2 or 1 amino acid difference(s) with SEQ ID NO: 13; and
b. a third ISVD and a fourth ISVD, each specifically binds to TNF-a and each comprises
iv. a CDR1 comprising the amino acid sequence of SEQ ID NO: 8 or has 2 or 1 amino acid difference(s) with SEQ ID NO: 8;
v. a CDR2 comprising the amino acid sequence of SEQ ID NO: 11 or has 2 or 1 amino acid difference(s) with SEQ ID NO: 11; and
vi. a CDR3 comprising the amino acid sequence of SEQ ID NO: 14 or has 2 or 1 amino acid difference(s) with SEQ ID NO: 14.

2. The polypeptide or composition according to claim 1, wherein:
a. said first ISVD and said second ISVD each comprise a CDR1 comprising the amino acid sequence of SEQ ID NO: 7, a CDR2 comprising the amino acid sequence of SEQ ID NO: 10 and a CDR3 comprising the amino acid sequence of SEQ ID NO: 13; and
b. said third ISVD and said fourth ISVD each comprise a CDR1 comprising the amino acid sequence of SEQ ID NO: 8, a CDR2 comprising the amino acid sequence of SEQ ID NO: 11 and a CDR3 comprising the amino acid sequence of SEQ ID NO: 14.

3. The polypeptide or composition according to claim 1, wherein:
a. the amino acid sequence of said first ISVD comprises a sequence identity of more than 90% with SEQ ID NO: 2;
b. the amino acid sequence of said second ISVD comprises a sequence identity of more than 90% with SEQ ID NO: 3;
c. the amino acid sequence of said third ISVD comprises a sequence identity of more than 90% identity with SEQ ID NO: 4; and
d. the amino acid sequence of said fourth ISVD comprises a sequence identity of more than 90% identity with SEQ ID NO: 6.

4. The polypeptide or composition according to claim 3, wherein:
a. said first ISVD comprises the amino acid sequence of SEQ ID NO: 2;
b. said second ISVD comprises the amino acid sequence of SEQ ID NO: 3;
c. said third ISVD comprises the amino acid sequence of SEQ ID NO: 4; and
d. said fourth ISVD comprises the amino acid sequence of SEQ ID NO: 6.

5. The polypeptide or composition according to claim 1, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

6. The polypeptide or composition according to claim 5, in which said one or more other groups, residues, moieties or binding units that provide the polypeptide with increased half-life is chosen from the group consisting of binding units that can bind to serum albumin (such as human serum albumin) or a serum immunoglobulin (such as IgG).

7. The polypeptide or composition according to claim 6, in which said binding unit that provides the polypeptide with increased half-life is an ISVD that can bind to human serum albumin.

8. The polypeptide or composition according to claim 7, wherein the ISVD binding to human serum albumin comprises
i. a CDR1 comprising the amino acid sequence of SEQ ID NO: 9 or has 2 or 1 amino acid difference(s) with SEQ ID NO: 9;
ii. a CDR2 comprising the amino acid sequence of SEQ ID NO: 12 or has 2 or 1 amino acid difference(s) with SEQ ID NO: 12; and
iii. a CDR3 comprising the amino acid sequence of SEQ ID NO: 15 or has 2 or 1 amino acid difference(s) with SEQ ID NO: 15.

9. The polypeptide or composition according to claim 8, wherein the ISVD binding to human serum albumin comprises a CDR1 comprising the amino acid sequence of SEQ ID NO: 9, a CDR2 comprising the amino acid sequence of SEQ ID NO: 12 and a CDR3 comprising the amino acid sequence of SEQ ID NO: 15.

10. The polypeptide or composition according to claim 8, wherein the amino acid sequence of said ISVD binding to human serum albumin comprises a sequence identity of more than 90% with SEQ ID NO: 5.

11. The polypeptide or composition according to claim 10, wherein said ISVD binding to human serum albumin comprises the amino acid sequence of SEQ ID NO: 5.

12. The polypeptide or composition according to claim 1, wherein the amino acid sequence of the polypeptide comprises a sequence identity of more than 90% with SEQ ID NO: 1.

13. The polypeptide or composition according to claim 12, wherein the polypeptide comprises or consists of the amino acid sequence of SEQ ID NO: 1.

14. A nucleic acid comprising a nucleotide sequence that encodes a polypeptide according to claim 1.

15. A host or host cell comprising a nucleic acid according to claim 14.

16. A method for producing a polypeptide, said method comprising the steps of:
a. expressing, in a suitable host cell or host organism or in another suitable expression system, a nucleic acid according to claim 14; optionally followed by:
b. isolating and/or purifying the polypeptide.

17. A composition comprising at least one polypeptide according to claim 1.

18. The composition according to claim 17, further comprising at least one pharmaceutically acceptable carrier, diluent or excipient and/or adjuvant, and optionally comprises one or more further pharmaceutically active polypeptides and/or compounds.

19.-21. (canceled)

22. A method of treating an autoimmune disease or an inflammatory disease, wherein said method comprises administering, to a subject in need thereof, a pharmaceutically active amount of a polypeptide according to claim 1.

23. The method according to claim 22, wherein the autoimmune disease or inflammatory disease is selected from rheumatoid arthritis, inflammatory bowel disease, such as Crohn's disease and ulcerative colitis, psoriasis, Hidradenitis suppurativa, and graft-versus-host disease.

24.-25. (canceled)
